SEQUENCE LISTING (651 sequences)

SEQ ID NO: 1 - DNA, 25 bp, synthetic construct
  misc_feature 1..25: note = Antisense sequence to target splice site of proprocessed human dystrophin
  source 1..25: mol_type = other DNA
ctgcaggtaaaagcatatggatcaa

SEQ ID NO: 2 - DNA, 25 bp, synthetic construct
  misc_feature 1..25: note = Antisense sequence to target splice site of proprocessed human dystrophin
  source 1..25: mol_type = other DNA
atcgcctgcaggtaaaagcatatgg

SEQ ID NO: 3 - DNA, 25 bp, synthetic construct
  misc_feature 1..25: note = Antisense sequence to target splice site of proprocessed human dystrophin
  source 1..25: mol_type = other DNA
gtcaaatcgcctgcaggtaaaagca

SEQ ID NO: 4 - DNA, 25 bp, synthetic construct
  misc_feature 1..25: note = Antisense sequence to target splice site of proprocessed human dystrophin
  source 1..25: mol_type = other DNA
gatctgtcaaatcgcctgcaggtaa

SEQ ID NO: 5 - DNA, 25 bp, synthetic construct
  misc_feature 1..25: note = Antisense sequence to target splice site of proprocessed human dystrophin
  source 1..25: mol_type = other DNA
caacagatctgtcaaatcgcctgca

SEQ ID NO: 6 - DNA, 25 bp, synthetic construct
  misc_feature 1..25: note = Antisense sequence to target splice site of proprocessed human dystrophin
  source 1..25: mol_type = other DNA
tttctcaacagatctgtcaaatcgc

SEQ ID NO: 7 - DNA, 25 bp, synthetic construct
  misc_feature 1..25: note = Antisense sequence to target splice site of proprocessed human dystrophin
  source 1..25: mol_type = other DNA
ccatttctcaacagatctgtcaaat

SEQ ID NO: 8 - DNA, 25 bp, synthetic construct
  misc_feature 1..25: note = Antisense sequence to target splice site of proprocessed human dystrophin
  source 1..25: mol_type = other DNA
ataatgaaaacgccgccatttctca

SEQ ID NO: 9 - DNA, 25 bp, synthetic construct
  misc_feature 1..25: note = Antisense sequence to target splice site of proprocessed human dystrophin
  source 1..25: mol_type = other DNA
aaatatctttatatcataatgaaaa

SEQ ID NO: 10 - DNA, 25 bp, synthetic construct
  misc_feature 1..25: note = Antisense sequence to target splice site of proprocessed human dystrophin
  source 1..25: mol_type = other DNA
tgttagccactgattaaatatcttt

SEQ ID NO: 11 - DNA, 25 bp, synthetic construct
  misc_feature 1..25: note = Antisense sequence to target splice site of proprocessed human dystrophin
  source 1..25: mol_type = other DNA
aaactgttcagcttctgttagccac

SEQ ID NO: 12 - DNA, 25 bp, synthetic construct
  misc_feature 1..25: note = Antisense sequence to target splice site of proprocessed human dystrophin
  source 1..25: mol_type = other DNA
ttgtgtctttctgagaaactgttca

SEQ ID NO: 13 - DNA, 25 bp, synthetic construct
  misc_feature 1..25: note = Antisense sequence to target splice site of proprocessed human dystrophin
  source 1..25: mol_type = other DNA
ccaattctcaggaatttgtgtcttt

SEQ ID NO: 14 - DNA, 25 bp, synthetic construct
  misc_feature 1..25: note = Antisense sequence to target splice site of proprocessed human dystrophin
  source 1..25: mol_type = other DNA
gtatttagcatgttcccaattctca

SEQ ID NO: 15 - DNA, 25 bp, synthetic construct
  misc_feature 1..25: note = Antisense sequence to target splice site of proprocessed human dystrophin
  source 1..25: mol_type = other DNA
cttaagataccatttgtatttagca

SEQ ID NO: 16 - DNA, 25 bp, synthetic construct
  misc_feature 1..25: note = Antisense sequence to target splice site of proprocessed human dystrophin
  source 1..25: mol_type = other DNA
cttaccttaagataccatttgtatt

SEQ ID NO: 17 - DNA, 25 bp, synthetic construct
  misc_feature 1..25: note = Antisense sequence to target splice site of proprocessed human dystrophin
  source 1..25: mol_type = other DNA
aaagacttaccttaagataccattt

SEQ ID NO: 18 - DNA, 25 bp, synthetic construct
  misc_feature 1..25: note = Antisense sequence to target splice site of proprocessed human dystrophin
  source 1..25: mol_type = other DNA
aaatcaaagacttaccttaagatac

SEQ ID NO: 19 - DNA, 25 bp, synthetic construct
  misc_feature 1..25: note = Antisense sequence to target splice site of proprocessed human dystrophin
  source 1..25: mol_type = other DNA
aaaacaaatcaaagacttaccttaa

SEQ ID NO: 20 - DNA, 25 bp, synthetic construct
  misc_feature 1..25: note = Antisense sequence to target splice site of proprocessed human dystrophin
  source 1..25: mol_type = other DNA
tcgaaaaaacaaatcaaagacttac

SEQ ID NO: 21 - DNA, 25 bp, synthetic construct
  misc_feature 1..25: note = Antisense sequence to target splice site of proprocessed human dystrophin
  source 1..25: mol_type = other DNA
ctgtaagataccaaaaaggcaaaac

SEQ ID NO: 22 - DNA, 25 bp, synthetic construct
  misc_feature 1..25: note = Antisense sequence to target splice site of proprocessed human dystrophin
  source 1..25: mol_type = other DNA
cctgtaagataccaaaaaggcaaaa

SEQ ID NO: 23 - DNA, 25 bp, synthetic construct
  misc_feature 1..25: note = Antisense sequence to target splice site of proprocessed human dystrophin
  source 1..25: mol_type = other DNA
agttcctgtaagataccaaaaaggc

SEQ ID NO: 24 - DNA, 25 bp, synthetic construct
  misc_feature 1..25: note = Antisense sequence to target splice site of proprocessed human dystrophin
  source 1..25: mol_type = other DNA
gagttcctgtaagataccaaaaagg

SEQ ID NO: 25 - DNA, 25 bp, synthetic construct
  misc_feature 1..25: note = Antisense sequence to target splice site of proprocessed human dystrophin
  source 1..25: mol_type = other DNA
cctggagttcctgtaagataccaaa

SEQ ID NO: 26 - DNA, 25 bp, synthetic construct
  misc_feature 1..25: note = Antisense sequence to target splice site of proprocessed human dystrophin
  source 1..25: mol_type = other DNA
tcctggagttcctgtaagataccaa

SEQ ID NO: 27 - DNA, 25 bp, synthetic construct
  misc_feature 1..25: note = Antisense sequence to target splice site of proprocessed human dystrophin
  source 1..25: mol_type = other DNA
gccatcctggagttcctgtaagata

SEQ ID NO: 28 - DNA, 25 bp, synthetic construct
  misc_feature 1..25: note = Antisense sequence to target splice site of proprocessed human dystrophin
  source 1..25: mol_type = other DNA
tgccatcctggagttcctgtaagat

SEQ ID NO: 29 - DNA, 25 bp, synthetic construct
  misc_feature 1..25: note = Antisense sequence to target splice site of proprocessed human dystrophin
  source 1..25: mol_type = other DNA
ccaatgccatcctggagttcctgta

SEQ ID NO: 30 - DNA, 25 bp, synthetic construct
  misc_feature 1..25: note = Antisense sequence to target splice site of proprocessed human dystrophin
  source 1..25: mol_type = other DNA
cccaatgccatcctggagttcctgt

SEQ ID NO: 31 - DNA, 25 bp, synthetic construct
  misc_feature 1..25: note = Antisense sequence to target splice site of proprocessed human dystrophin
  source 1..25: mol_type = other DNA
gctgcccaatgccatcctggagttc

SEQ ID NO: 32 - DNA, 25 bp, synthetic construct
  misc_feature 1..25: note = Antisense sequence to target splice site of proprocessed human dystrophin
  source 1..25: mol_type = other DNA
cgctgcccaatgccatcctggagtt

SEQ ID NO: 33 - DNA, 25 bp, synthetic construct
  misc_feature 1..25: note = Antisense sequence to target splice site of proprocessed human dystrophin
  source 1..25: mol_type = other DNA
aacagtttgccgctgcccaatgcca

SEQ ID NO: 34 - DNA, 25 bp, synthetic construct
  misc_feature 1..25: note = Antisense sequence to target splice site of proprocessed human dystrophin
  source 1..25: mol_type = other DNA
ctgacaacagtttgccgctgcccaa

SEQ ID NO: 35 - DNA, 25 bp, synthetic construct
  misc_feature 1..25: note = Antisense sequence to target splice site of proprocessed human dystrophin
  source 1..25: mol_type = other DNA
gttgcattcaatgttctgacaacag

SEQ ID NO: 36 - DNA, 25 bp, synthetic construct
  misc_feature 1..25: note = Antisense sequence to target splice site of proprocessed human dystrophin
  source 1..25: mol_type = other DNA
gctgaattatttcttccccagttgc

SEQ ID NO: 37 - DNA, 25 bp, synthetic construct
  misc_feature 1..25: note = Antisense sequence to target splice site of proprocessed human dystrophin
  source 1..25: mol_type = other DNA
attatttcttccccagttgcattca

SEQ ID NO: 38 - DNA, 25 bp, synthetic construct
  misc_feature 1..25: note = Antisense sequence to target splice site of proprocessed human dystrophin
  source 1..25: mol_type = other DNA
ggcatctgtttttgaggattgctga

SEQ ID NO: 39 - DNA, 25 bp, synthetic construct
  misc_feature 1..25: note = Antisense sequence to target splice site of proprocessed human dystrophin
  source 1..25: mol_type = other DNA
tttgaggattgctgaattatttctt

SEQ ID NO: 40 - DNA, 25 bp, synthetic construct
  misc_feature 1..25: note = Antisense sequence to target splice site of proprocessed human dystrophin
  source 1..25: mol_type = other DNA
aatttttcctgtagaatactggcat

SEQ ID NO: 41 - DNA, 25 bp, synthetic construct
  misc_feature 1..25: note = Antisense sequence to target splice site of proprocessed human dystrophin
  source 1..25: mol_type = other DNA
atactggcatctgtttttgaggatt

SEQ ID NO: 42 - DNA, 25 bp, synthetic construct
  misc_feature 1..25: note = Antisense sequence to target splice site of proprocessed human dystrophin
  source 1..25: mol_type = other DNA
accgcagattcaggcttcccaattt

SEQ ID NO: 43 - DNA, 25 bp, synthetic construct
  misc_feature 1..25: note = Antisense sequence to target splice site of proprocessed human dystrophin
  source 1..25: mol_type = other DNA
aatttttcctgtagaatactggcat

SEQ ID NO: 44 - DNA, 25 bp, synthetic construct
  misc_feature 1..25: note = Antisense sequence to target splice site of proprocessed human dystrophin
  source 1..25: mol_type = other DNA
ctgtttgcagacctcctgccaccgc

SEQ ID NO: 45 - DNA, 25 bp, synthetic construct
  misc_feature 1..25: note = Antisense sequence to target splice site of proprocessed human dystrophin
  source 1..25: mol_type = other DNA
agattcaggcttcccaatttttcct

SEQ ID NO: 46 - DNA, 25 bp, synthetic construct
  misc_feature 1..25: note = Antisense sequence to target splice site of proprocessed human dystrophin
  source 1..25: mol_type = other DNA
ctcttttttctgtctgacagctgtt

SEQ ID NO: 47 - DNA, 25 bp, synthetic construct
  misc_feature 1..25: note = Antisense sequence to target splice site of proprocessed human dystrophin
  source 1..25: mol_type = other DNA
acctcctgccaccgcagattcaggc

SEQ ID NO: 48 - DNA, 25 bp, synthetic construct
  misc_feature 1..25: note = Antisense sequence to target splice site of proprocessed human dystrophin
  source 1..25: mol_type = other DNA
cctacctcttttttctgtctgacag

SEQ ID NO: 49 - DNA, 25 bp, synthetic construct
  misc_feature 1..25: note = Antisense sequence to target splice site of proprocessed human dystrophin
  source 1..25: mol_type = other DNA
gacagctgtttgcagacctcctgcc

SEQ ID NO: 50 - DNA, 25 bp, synthetic construct
  misc_feature 1..25: note = Antisense sequence to target splice site of proprocessed human dystrophin
  source 1..25: mol_type = other DNA
gtcgccctacctcttttttctgtct

SEQ ID NO: 51 - DNA, 25 bp, synthetic construct
  misc_feature 1..25: note = Antisense sequence to target splice site of proprocessed human dystrophin
  source 1..25: mol_type = other DNA
gatctgtcgccctacctcttttttc

SEQ ID NO: 52 - DNA, 25 bp, synthetic construct
  misc_feature 1..25: note = Antisense sequence to target splice site of proprocessed human dystrophin
  source 1..25: mol_type = other DNA
tattagatctgtcgccctacctctt

SEQ ID NO: 53 - DNA, 25 bp, synthetic construct
  misc_feature 1..25: note = Antisense sequence to target splice site of proprocessed human dystrophin
  source 1..25: mol_type = other DNA
attcctattagatctgtcgccctac

SEQ ID NO: 54 - DNA, 20 bp, synthetic construct
  misc_feature 1..20: note = Antisense sequence to target splice site of proprocessed human dystrophin
  source 1..20: mol_type = other DNA
agataccaaaaaggcaaaac

SEQ ID NO: 55 - DNA, 20 bp, synthetic construct
  misc_feature 1..20: note = Antisense sequence to target splice site of proprocessed human dystrophin
  source 1..20: mol_type = other DNA
aagataccaaaaaggcaaaa

SEQ ID NO: 56 - DNA, 20 bp, synthetic construct
  misc_feature 1..20: note = Antisense sequence to target splice site of proprocessed human dystrophin
  source 1..20: mol_type = other DNA
cctgtaagataccaaaaagg

SEQ ID NO: 57 - DNA, 20 bp, synthetic construct
  misc_feature 1..20: note = Antisense sequence to target splice site of proprocessed human dystrophin
  source 1..20: mol_type = other DNA
gagttcctgtaagataccaa

SEQ ID NO: 58 - DNA, 20 bp, synthetic construct
  misc_feature 1..20: note = Antisense sequence to target splice site of proprocessed human dystrophin
  source 1..20: mol_type = other DNA
tcctggagttcctgtaagat

SEQ ID NO: 59 - DNA, 20 bp, synthetic construct
  misc_feature 1..20: note = Antisense sequence to target splice site of proprocessed human dystrophin
  source 1..20: mol_type = other DNA
tgccatcctggagttcctgt

SEQ ID NO: 60 - DNA, 20 bp, synthetic construct
  misc_feature 1..20: note = Antisense sequence to target splice site of proprocessed human dystrophin
  source 1..20: mol_type = other DNA
cccaatgccatcctggagtt

SEQ ID NO: 61 - DNA, 20 bp, synthetic construct
  misc_feature 1..20: note = Antisense sequence to target splice site of proprocessed human dystrophin
  source 1..20: mol_type = other DNA
cgctgcccaatgccatcctg

SEQ ID NO: 62 - DNA, 20 bp, synthetic construct
  misc_feature 1..20: note = Antisense sequence to target splice site of proprocessed human dystrophin
  source 1..20: mol_type = other DNA
ctgacaacagtttgccgctg

SEQ ID NO: 63 - DNA, 20 bp, synthetic construct
  misc_feature 1..20: note = Antisense sequence to target splice site of proprocessed human dystrophin
  source 1..20: mol_type = other DNA
gttgcattcaatgttctgac

SEQ ID NO: 64 - DNA, 20 bp, synthetic construct
  misc_feature 1..20: note = Antisense sequence to target splice site of proprocessed human dystrophin
  source 1..20: mol_type = other DNA
attatttcttccccagttgc

SEQ ID NO: 65 - DNA, 20 bp, synthetic construct
  misc_feature 1..20: note = Antisense sequence to target splice site of proprocessed human dystrophin
  source 1..20: mol_type = other DNA
tttgaggattgctgaattat

SEQ ID NO: 66 - DNA, 20 bp, synthetic construct
  misc_feature 1..20: note = Antisense sequence to target splice site of proprocessed human dystrophin
  source 1..20: mol_type = other DNA
atactggcatctgtttttga

SEQ ID NO: 67 - DNA, 20 bp, synthetic construct
  misc_feature 1..20: note = Antisense sequence to target splice site of proprocessed human dystrophin
  source 1..20: mol_type = other DNA
aatttttcctgtagaatact

SEQ ID NO: 68 - DNA, 20 bp, synthetic construct
  misc_feature 1..20: note = Antisense sequence to target splice site of proprocessed human dystrophin
  source 1..20: mol_type = other DNA
agattcaggcttcccaattt

SEQ ID NO: 69 - DNA, 20 bp, synthetic construct
  misc_feature 1..20: note = Antisense sequence to target splice site of proprocessed human dystrophin
  source 1..20: mol_type = other DNA
acctcctgccaccgcagatt

SEQ ID NO: 70 - DNA, 20 bp, synthetic construct
  misc_feature 1..20: note = Antisense sequence to target splice site of proprocessed human dystrophin
  source 1..20: mol_type = other DNA
gacagctgtttgcagacctc

SEQ ID NO: 71 - DNA, 20 bp, synthetic construct
  misc_feature 1..20: note = Antisense sequence to target splice site of proprocessed human dystrophin
  source 1..20: mol_type = other DNA
ctcttttttctgtctgacag

SEQ ID NO: 72 - DNA, 20 bp, synthetic construct
  misc_feature 1..20: note = Antisense sequence to target splice site of proprocessed human dystrophin
  source 1..20: mol_type = other DNA
cctacctcttttttctgtct

SEQ ID NO: 73 - DNA, 20 bp, synthetic construct
  misc_feature 1..20: note = Antisense sequence to target splice site of proprocessed human dystrophin
  source 1..20: mol_type = other DNA
gtcgccctacctcttttttc

SEQ ID NO: 74 - DNA, 20 bp, synthetic construct
  misc_feature 1..20: note = Antisense sequence to target splice site of proprocessed human dystrophin
  source 1..20: mol_type = other DNA
gatctgtcgccctacctctt

SEQ ID NO: 75 - DNA, 20 bp, synthetic construct
  misc_feature 1..20: note = Antisense sequence to target splice site of proprocessed human dystrophin
  source 1..20: mol_type = other DNA
tattagatctgtcgccctac

SEQ ID NO: 76 - DNA, 20 bp, synthetic construct
  misc_feature 1..20: note = Antisense sequence to target splice site of proprocessed human dystrophin
  source 1..20: mol_type = other DNA
attcctattagatctgtcgc

SEQ ID NO: 77 - DNA, 25 bp, synthetic construct
  misc_feature 1..25: note = Antisense sequence to target splice site of proprocessed human dystrophin
  source 1..25: mol_type = other DNA
gggggatttgagaaaataaaattac

SEQ ID NO: 78 - DNA, 25 bp, synthetic construct
  misc_feature 1..25: note = Antisense sequence to target splice site of proprocessed human dystrophin
  source 1..25: mol_type = other DNA
atttgagaaaataaaattaccttga

SEQ ID NO: 79 - DNA, 25 bp, synthetic construct
  misc_feature 1..25: note = Antisense sequence to target splice site of proprocessed human dystrophin
  source 1..25: mol_type = other DNA
ctagcctggagaaagaagaataaaa

SEQ ID NO: 80 - DNA, 25 bp, synthetic construct
  misc_feature 1..25: note = Antisense sequence to target splice site of proprocessed human dystrophin
  source 1..25: mol_type = other DNA
agaaaataaaattaccttgacttgc

SEQ ID NO: 81 - DNA, 25 bp, synthetic construct
  misc_feature 1..25: note = Antisense sequence to target splice site of proprocessed human dystrophin
  source 1..25: mol_type = other DNA
ttcttctagcctggagaaagaagaa

SEQ ID NO: 82 - DNA, 25 bp, synthetic construct
  misc_feature 1..25: note = Antisense sequence to target splice site of proprocessed human dystrophin
  source 1..25: mol_type = other DNA
ataaaattaccttgacttgctcaag

SEQ ID NO: 83 - DNA, 25 bp, synthetic construct
  misc_feature 1..25: note = Antisense sequence to target splice site of proprocessed human dystrophin
  source 1..25: mol_type = other DNA
ttttgttcttctagcctggagaaag

SEQ ID NO: 84 - DNA, 25 bp, synthetic construct
  misc_feature 1..25: note = Antisense sequence to target splice site of proprocessed human dystrophin
  source 1..25: mol_type = other DNA
attaccttgacttgctcaagctttt

SEQ ID NO: 85 - DNA, 25 bp, synthetic construct
  misc_feature 1..25: note = Antisense sequence to target splice site of proprocessed human dystrophin
  source 1..25: mol_type = other DNA
tattcttttgttcttctagcctgga

SEQ ID NO: 86 - DNA, 25 bp, synthetic construct
  misc_feature 1..25: note = Antisense sequence to target splice site of proprocessed human dystrophin
  source 1..25: mol_type = other DNA
cttgacttgctcaagcttttctttt

SEQ ID NO: 87 - DNA, 25 bp, synthetic construct
  misc_feature 1..25: note = Antisense sequence to target splice site of proprocessed human dystrophin
  source 1..25: mol_type = other DNA
caagatattcttttgttcttctagc

SEQ ID NO: 88 - DNA, 25 bp, synthetic construct
  misc_feature 1..25: note = Antisense sequence to target splice site of proprocessed human dystrophin
  source 1..25: mol_type = other DNA
cttttagttgctgctcttttccagg

SEQ ID NO: 89 - DNA, 25 bp, synthetic construct
  misc_feature 1..25: note = Antisense sequence to target splice site of proprocessed human dystrophin
  source 1..25: mol_type = other DNA
ccaggttcaagtgggatactagcaa

SEQ ID NO: 90 - DNA, 25 bp, synthetic construct
  misc_feature 1..25: note = Antisense sequence to target splice site of proprocessed human dystrophin
  source 1..25: mol_type = other DNA
atctctttgaaattctgacaagata

SEQ ID NO: 91 - DNA, 25 bp, synthetic construct
  misc_feature 1..25: note = Antisense sequence to target splice site of proprocessed human dystrophin
  source 1..25: mol_type = other DNA
agcaatgttatctgcttcctccaac

SEQ ID NO: 92 - DNA, 25 bp, synthetic construct
  misc_feature 1..25: note = Antisense sequence to target splice site of proprocessed human dystrophin
  source 1..25: mol_type = other DNA
aacaaattcatttaaatctctttga

SEQ ID NO: 93 - DNA, 25 bp, synthetic construct
  misc_feature 1..25: note = Antisense sequence to target splice site of proprocessed human dystrophin
  source 1..25: mol_type = other DNA
ccaaccataaaacaaattcatttaa

SEQ ID NO: 94 - DNA, 25 bp, synthetic construct
  misc_feature 1..25: note = Antisense sequence to target splice site of proprocessed human dystrophin
  source 1..25: mol_type = other DNA
ttcctccaaccataaaacaaattca

SEQ ID NO: 95 - DNA, 25 bp, synthetic construct
  misc_feature 1..25: note = Antisense sequence to target splice site of proprocessed human dystrophin
  source 1..25: mol_type = other DNA
tttaaatctctttgaaattctgaca

SEQ ID NO: 96 - DNA, 25 bp, synthetic construct
  misc_feature 1..25: note = Antisense sequence to target splice site of proprocessed human dystrophin
  source 1..25: mol_type = other DNA
tgacaagatattcttttgttcttct

SEQ ID NO: 97 - DNA, 25 bp, synthetic construct
  misc_feature 1..25: note = Antisense sequence to target splice site of proprocessed human dystrophin
  source 1..25: mol_type = other DNA
ttcaagtgggatactagcaatgtta

SEQ ID NO: 98 - DNA, 25 bp, synthetic construct
  misc_feature 1..25: note = Antisense sequence to target splice site of proprocessed human dystrophin
  source 1..25: mol_type = other DNA
agatattcttttgttcttctagcct

SEQ ID NO: 99 - DNA, 25 bp, synthetic construct
  misc_feature 1..25: note = Antisense sequence to target splice site of proprocessed human dystrophin
  source 1..25: mol_type = other DNA
ctgctcttttccaggttcaagtggg

SEQ ID NO: 100 - DNA, 25 bp, synthetic construct
  misc_feature 1..25: note = Antisense sequence to target splice site of proprocessed human dystrophin
  source 1..25: mol_type = other DNA
ttcttttgttcttctagcctggaga

SEQ ID NO: 101 - DNA, 25 bp, synthetic construct
  misc_feature 1..25: note = Antisense sequence to target splice site of proprocessed human dystrophin
  source 1..25: mol_type = other DNA
cttttcttttagttgctgctctttt

SEQ ID NO: 102 - DNA, 25 bp, synthetic construct
  misc_feature 1..25: note = Antisense sequence to target splice site of proprocessed human dystrophin
  source 1..25: mol_type = other DNA
ttgttcttctagcctggagaaagaa

SEQ ID NO: 103 - DNA, 25 bp, synthetic construct
  misc_feature 1..25: note = Antisense sequence to target splice site of proprocessed human dystrophin
  source 1..25: mol_type = other DNA
cttctagcctggagaaagaagaata

SEQ ID NO: 104 - DNA, 25 bp, synthetic construct
  misc_feature 1..25: note = Antisense sequence to target splice site of proprocessed human dystrophin
  source 1..25: mol_type = other DNA
agcctggagaaagaagaataaaatt

SEQ ID NO: 105 - DNA, 25 bp, synthetic construct
  misc_feature 1..25: note = Antisense sequence to target splice site of proprocessed human dystrophin
  source 1..25: mol_type = other DNA
ctggagaaagaagaataaaattgtt

SEQ ID NO: 106 - DNA, 20 bp, synthetic construct
  misc_feature 1..20: note = Antisense sequence to target splice site of proprocessed human dystrophin
  source 1..20: mol_type = other DNA
gaaagaagaataaaattgtt

SEQ ID NO: 107 - DNA, 20 bp, synthetic construct
  misc_feature 1..20: note = Antisense sequence to target splice site of proprocessed human dystrophin
  source 1..20: mol_type = other DNA
ggagaaagaagaataaaatt

SEQ ID NO: 108 - DNA, 20 bp, synthetic construct
  misc_feature 1..20: note = Antisense sequence to target splice site of proprocessed human dystrophin
  source 1..20: mol_type = other DNA
agcctggagaaagaagaata

SEQ ID NO: 109 - DNA, 20 bp, synthetic construct
  misc_feature 1..20: note = Antisense sequence to target splice site of proprocessed human dystrophin
  source 1..20: mol_type = other DNA
cttctagcctggagaaagaa

SEQ ID NO: 110 - DNA, 20 bp, synthetic construct
  misc_feature 1..20: note = Antisense sequence to target splice site of proprocessed human dystrophin
  source 1..20: mol_type = other DNA
ttgttcttctagcctggaga

SEQ ID NO: 111 - DNA, 20 bp, synthetic construct
  misc_feature 1..20: note = Antisense sequence to target splice site of proprocessed human dystrophin
  source 1..20: mol_type = other DNA
ttcttttgttcttctagcct

SEQ ID NO: 112 - DNA, 20 bp, synthetic construct
  misc_feature 1..20: note = Antisense sequence to target splice site of proprocessed human dystrophin
  source 1..20: mol_type = other DNA
tgacaagatattcttttgtt

SEQ ID NO: 113 - DNA, 20 bp, synthetic construct
  misc_feature 1..20: note = Antisense sequence to target splice site of proprocessed human dystrophin
  source 1..20: mol_type = other DNA
atctctttgaaattctgaca

SEQ ID NO: 114 - DNA, 20 bp, synthetic construct
  misc_feature 1..20: note = Antisense sequence to target splice site of proprocessed human dystrophin
  source 1..20: mol_type = other DNA
aacaaattcatttaaatctc

SEQ ID NO: 115 - DNA, 20 bp, synthetic construct
  misc_feature 1..20: note = Antisense sequence to target splice site of proprocessed human dystrophin
  source 1..20: mol_type = other DNA
ttcctccaaccataaaacaa

SEQ ID NO: 116 - DNA, 20 bp, synthetic construct
  misc_feature 1..20: note = Antisense sequence to target splice site of proprocessed human dystrophin
  source 1..20: mol_type = other DNA
agcaatgttatctgcttcct

SEQ ID NO: 117 - DNA, 20 bp, synthetic construct
  misc_feature 1..20: note = Antisense sequence to target splice site of proprocessed human dystrophin
  source 1..20: mol_type = other DNA
ttcaagtgggatactagcaa

SEQ ID NO: 118 - DNA, 20 bp, synthetic construct
  misc_feature 1..20: note = Antisense sequence to target splice site of proprocessed human dystrophin
  source 1..20: mol_type = other DNA
ctgctcttttccaggttcaa

SEQ ID NO: 119 - DNA, 20 bp, synthetic construct
  misc_feature 1..20: note = Antisense sequence to target splice site of proprocessed human dystrophin
  source 1..20: mol_type = other DNA
cttttcttttagttgctgct

SEQ ID NO: 120 - DNA, 20 bp, synthetic construct
  misc_feature 1..20: note = Antisense sequence to target splice site of proprocessed human dystrophin
  source 1..20: mol_type = other DNA
cttgacttgctcaagctttt

SEQ ID NO: 121 - DNA, 20 bp, synthetic construct
  misc_feature 1..20: note = Antisense sequence to target splice site of proprocessed human dystrophin
  source 1..20: mol_type = other DNA
attaccttgacttgctcaag

SEQ ID NO: 122 - DNA, 20 bp, synthetic construct
  misc_feature 1..20: note = Antisense sequence to target splice site of proprocessed human dystrophin
  source 1..20: mol_type = other DNA
ataaaattaccttgacttgc

SEQ ID NO: 123 - DNA, 20 bp, synthetic construct
  misc_feature 1..20: note = Antisense sequence to target splice site of proprocessed human dystrophin
  source 1..20: mol_type = other DNA
agaaaataaaattaccttga

SEQ ID NO: 124 - DNA, 20 bp, synthetic construct
  misc_feature 1..20: note = Antisense sequence to target splice site of proprocessed human dystrophin
  source 1..20: mol_type = other DNA
atttgagaaaataaaattac

SEQ ID NO: 125 - DNA, 20 bp, synthetic construct
  misc_feature 1..20: note = Antisense sequence to target splice site of proprocessed human dystrophin
  source 1..20: mol_type = other DNA
gggggatttgagaaaataaa

SEQ ID NO: 126 - DNA, 25 bp, synthetic construct
  misc_feature 1..25: note = Antisense sequence to target splice site of proprocessed human dystrophin
  source 1..25: mol_type = other DNA
ctgaaacagacaaatgcaacaacgt

SEQ ID NO: 127 - DNA, 25 bp, synthetic construct
  misc_feature 1..25: note = Antisense sequence to target splice site of proprocessed human dystrophin
  source 1..25: mol_type = other DNA
agtaactgaaacagacaaatgcaac

SEQ ID NO: 128 - DNA, 25 bp, synthetic construct
  misc_feature 1..25: note = Antisense sequence to target splice site of proprocessed human dystrophin
  source 1..25: mol_type = other DNA
ccaccagtaactgaaacagacaaat

SEQ ID NO: 129 - DNA, 25 bp, synthetic construct
  misc_feature 1..25: note = Antisense sequence to target splice site of proprocessed human dystrophin
  source 1..25: mol_type = other DNA
ctcttccaccagtaactgaaacaga

SEQ ID NO: 130 - DNA, 25 bp, synthetic construct
  misc_feature 1..25: note = Antisense sequence to target splice site of proprocessed human dystrophin
  source 1..25: mol_type = other DNA
ggcaactcttccaccagtaactgaa

SEQ ID NO: 131 - DNA, 25 bp, synthetic construct
  misc_feature 1..25: note = Antisense sequence to target splice site of proprocessed human dystrophin
  source 1..25: mol_type = other DNA
gcaggggcaactcttccaccagtaa

SEQ ID NO: 132 - DNA, 25 bp, synthetic construct
  misc_feature 1..25: note = Antisense sequence to target splice site of proprocessed human dystrophin
  source 1..25: mol_type = other DNA
ctggcgcaggggcaactcttccacc

SEQ ID NO: 133 - DNA, 25 bp, synthetic construct
  misc_feature 1..25: note = Antisense sequence to target splice site of proprocessed human dystrophin
  source 1..25: mol_type = other DNA
tttaattgtttgagaattccctggc

SEQ ID NO: 134 - DNA, 25 bp, synthetic construct
  misc_feature 1..25: note = Antisense sequence to target splice site of proprocessed human dystrophin
  source 1..25: mol_type = other DNA
ttgtttgagaattccctggcgcagg

SEQ ID NO: 135 - DNA, 25 bp, synthetic construct
  misc_feature 1..25: note = Antisense sequence to target splice site of proprocessed human dystrophin
  source 1..25: mol_type = other DNA
gcacgggtcctccagtttcatttaa

SEQ ID NO: 136 - DNA, 25 bp, synthetic construct
  misc_feature 1..25: note = Antisense sequence to target splice site of proprocessed human dystrophin
  source 1..25: mol_type = other DNA
tccagtttcatttaattgtttgaga

SEQ ID NO: 137 - DNA, 25 bp, synthetic construct
  misc_feature 1..25: note = Antisense sequence to target splice site of proprocessed human dystrophin
  source 1..25: mol_type = other DNA
gcttatgggagcacttacaagcacg

SEQ ID NO: 138 - DNA, 25 bp, synthetic construct
  misc_feature 1..25: note = Antisense sequence to target splice site of proprocessed human dystrophin
  source 1..25: mol_type = other DNA
tacaagcacgggtcctccagtttca

SEQ ID NO: 139 - DNA, 25 bp, synthetic construct
  misc_feature 1..25: note = Antisense sequence to target splice site of proprocessed human dystrophin
  source 1..25: mol_type = other DNA
agtttatcttgctcttctgggctta

SEQ ID NO: 140 - DNA, 25 bp, synthetic construct
  misc_feature 1..25: note = Antisense sequence to target splice site of proprocessed human dystrophin
  source 1..25: mol_type = other DNA
tctgcttgagcttattttcaagttt

SEQ ID NO: 141 - DNA, 25 bp, synthetic construct
  misc_feature 1..25: note = Antisense sequence to target splice site of proprocessed human dystrophin
  source 1..25: mol_type = other DNA
atcttgctcttctgggcttatggga

SEQ ID NO: 142 - DNA, 25 bp, synthetic construct
  misc_feature 1..25: note = Antisense sequence to target splice site of proprocessed human dystrophin
  source 1..25: mol_type = other DNA
ctttatccactggagatttgtctgc

SEQ ID NO: 143 - DNA, 25 bp, synthetic construct
  misc_feature 1..25: note = Antisense sequence to target splice site of proprocessed human dystrophin
  source 1..25: mol_type = other DNA
cttattttcaagtttatcttgctct

SEQ ID NO: 144 - DNA, 25 bp, synthetic construct
  misc_feature 1..25: note = Antisense sequence to target splice site of proprocessed human dystrophin
  source 1..25: mol_type = other DNA
ctaacctttatccactggagatttg

SEQ ID NO: 145 - DNA, 25 bp, synthetic construct
  misc_feature 1..25: note = Antisense sequence to target splice site of proprocessed human dystrophin
  source 1..25: mol_type = other DNA
atttgtctgcttgagcttattttca

SEQ ID NO: 146 - DNA, 25 bp, synthetic construct
  misc_feature 1..25: note = Antisense sequence to target splice site of proprocessed human dystrophin
  source 1..25: mol_type = other DNA
aatgtctaacctttatccactggag

SEQ ID NO: 147 - DNA, 25 bp, synthetic construct
  misc_feature 1..25: note = Antisense sequence to target splice site of proprocessed human dystrophin
  source 1..25: mol_type = other DNA
tggttaatgtctaacctttatccac

SEQ ID NO: 148 - DNA, 25 bp, synthetic construct
  misc_feature 1..25: note = Antisense sequence to target splice site of proprocessed human dystrophin
  source 1..25: mol_type = other DNA
agagatggttaatgtctaaccttta

SEQ ID NO: 149 - DNA, 25 bp, synthetic construct
  misc_feature 1..25: note = Antisense sequence to target splice site of proprocessed human dystrophin
  source 1..25: mol_type = other DNA
acggaagagatggttaatgtctaac

SEQ ID NO: 150 - DNA, 20 bp, synthetic construct
  misc_feature 1..20: note = Antisense sequence to target splice site of proprocessed human dystrophin
  source 1..20: mol_type = other DNA
acagacaaatgcaacaacgt

SEQ ID NO: 151 - DNA, 20 bp, synthetic construct
  misc_feature 1..20: note = Antisense sequence to target splice site of proprocessed human dystrophin
  source 1..20: mol_type = other DNA
ctgaaacagacaaatgcaac

SEQ ID NO: 152 - DNA, 20 bp, synthetic construct
  misc_feature 1..20: note = Antisense sequence to target splice site of proprocessed human dystrophin
  source 1..20: mol_type = other DNA
agtaactgaaacagacaaat

SEQ ID NO: 153 - DNA, 20 bp, synthetic construct
  misc_feature 1..20: note = Antisense sequence to target splice site of proprocessed human dystrophin
  source 1..20: mol_type = other DNA
ccaccagtaactgaaacaga

SEQ ID NO: 154 - DNA, 20 bp, synthetic construct
  misc_feature 1..20: note = Antisense sequence to target splice site of proprocessed human dystrophin
  source 1..20: mol_type = other DNA
ctcttccaccagtaactgaa

SEQ ID NO: 155 - DNA, 20 bp, synthetic construct
  misc_feature 1..20: note = Antisense sequence to target splice site of proprocessed human dystrophin
  source 1..20: mol_type = other DNA
ggcaactcttccaccagtaa

SEQ ID NO: 156 - DNA, 20 bp, synthetic construct
  misc_feature 1..20: note = Antisense sequence to target splice site of proprocessed human dystrophin
  source 1..20: mol_type = other DNA
ctggcgcaggggcaactctt

SEQ ID NO: 157 - DNA, 20 bp, synthetic construct
  misc_feature 1..20: note = Antisense sequence to target splice site of proprocessed human dystrophin
  source 1..20: mol_type = other DNA
ttgtttgagaattccctggc

SEQ ID NO: 158 - DNA, 20 bp, synthetic construct
  misc_feature 1..20: note = Antisense sequence to target splice site of proprocessed human dystrophin
  source 1..20: mol_type = other DNA
tccagtttcatttaattgtt

SEQ ID NO: 159 - DNA, 20 bp, synthetic construct
  misc_feature 1..20: note = Antisense sequence to target splice site of proprocessed human dystrophin
  source 1..20: mol_type = other DNA
tacaagcacgggtcctccag

SEQ ID NO: 160 - DNA, 20 bp, synthetic construct
  misc_feature 1..20: note = Antisense sequence to target splice site of proprocessed human dystrophin
  source 1..20: mol_type = other DNA
gcttatgggagcacttacaa

SEQ ID NO: 161 - DNA, 20 bp, synthetic construct
  misc_feature 1..20: note = Antisense sequence to target splice site of proprocessed human dystrophin
  source 1..20: mol_type = other DNA
atcttgctcttctgggctta

SEQ ID NO: 162 - DNA, 20 bp, synthetic construct
  misc_feature 1..20: note = Antisense sequence to target splice site of proprocessed human dystrophin
  source 1..20: mol_type = other DNA
cttattttcaagtttatctt

SEQ ID NO: 163 - DNA, 20 bp, synthetic construct
  misc_feature 1..20: note = Antisense sequence to target splice site of proprocessed human dystrophin
  source 1..20: mol_type = other DNA
atttgtctgcttgagcttat

SEQ ID NO: 164 - DNA, 20 bp, synthetic construct
  misc_feature 1..20: note = Antisense sequence to target splice site of proprocessed human dystrophin
  source 1..20: mol_type = other DNA
ctttatccactggagatttg

SEQ ID NO: 165 - DNA, 20 bp, synthetic construct
  misc_feature 1..20: note = Antisense sequence to target splice site of proprocessed human dystrophin
  source 1..20: mol_type = other DNA
ctaacctttatccactggag

SEQ ID NO: 166 - DNA, 20 bp, synthetic construct
  misc_feature 1..20: note = Antisense sequence to target splice site of proprocessed human dystrophin
  source 1..20: mol_type = other DNA
aatgtctaacctttatccac

SEQ ID NO: 167 - DNA, 20 bp, synthetic construct
  misc_feature 1..20: note = Antisense sequence to target splice site of proprocessed human dystrophin
  source 1..20: mol_type = other DNA
tggttaatgtctaaccttta

SEQ ID NO: 168 - DNA, 20 bp, synthetic construct
  misc_feature 1..20: note = Antisense sequence to target splice site of proprocessed human dystrophin
  source 1..20: mol_type = other DNA
agagatggttaatgtctaac

SEQ ID NO: 169 - DNA, 20 bp, synthetic construct
  misc_feature 1..20: note = Antisense sequence to target splice site of proprocessed human dystrophin
  source 1..20: mol_type = other DNA
acggaagagatggttaatgt

SEQ ID NO: 170 - DNA, 25 bp, synthetic construct
  misc_feature 1..25: note = Antisense sequence to target splice site of proprocessed human dystrophin
  source 1..25: mol_type = other DNA
ctgaaaggaaaatacattttaaaaa

SEQ ID NO: 171 - DNA, 25 bp, synthetic construct
  misc_feature 1..25: note = Antisense sequence to target splice site of proprocessed human dystrophin
  source 1..25: mol_type = other DNA
cctgaaaggaaaatacattttaaaa

SEQ ID NO: 172 - DNA, 25 bp, synthetic construct
  misc_feature 1..25: note = Antisense sequence to target splice site of proprocessed human dystrophin
  source 1..25: mol_type = other DNA
gaaacctgaaaggaaaatacatttt

SEQ ID NO: 173 - DNA, 25 bp, synthetic construct
  misc_feature 1..25: note = Antisense sequence to target splice site of proprocessed human dystrophin
  source 1..25: mol_type = other DNA
ggaaacctgaaaggaaaatacattt

SEQ ID NO: 174 - DNA, 25 bp, synthetic construct
  misc_feature 1..25: note = Antisense sequence to target splice site of proprocessed human dystrophin
  source 1..25: mol_type = other DNA
ctctggaaacctgaaaggaaaatac

SEQ ID NO: 175 - DNA, 25 bp, synthetic construct
  misc_feature 1..25: note = Antisense sequence to target splice site of proprocessed human dystrophin
  source 1..25: mol_type = other DNA
gctctggaaacctgaaaggaaaata

SEQ ID NO: 176 - DNA, 25 bp, synthetic construct
  misc_feature 1..25: note = Antisense sequence to target splice site of proprocessed human dystrophin
  source 1..25: mol_type = other DNA
gtaaagctctggaaacctgaaagga

SEQ ID NO: 177 - DNA, 25 bp, synthetic construct
  misc_feature 1..25: note = Antisense sequence to target splice site of proprocessed human dystrophin
  source 1..25: mol_type = other DNA
tcaggtaaagctctggaaacctgaa

SEQ ID NO: 178 - DNA, 25 bp, synthetic construct
  misc_feature 1..25: note = Antisense sequence to target splice site of proprocessed human dystrophin
  source 1..25: mol_type = other DNA
ctcaggtaaagctctggaaacctga

SEQ ID NO: 179 - DNA, 25 bp, synthetic construct
  misc_feature 1..25: note = Antisense sequence to target splice site of proprocessed human dystrophin
  source 1..25: mol_type = other DNA
gtttctcaggtaaagctctggaaac

SEQ ID NO: 180 - DNA, 25 bp, synthetic construct
  misc_feature 1..25: note = Antisense sequence to target splice site of proprocessed human dystrophin
  source 1..25: mol_type = other DNA
tgtttctcaggtaaagctctggaaa

SEQ ID NO: 181 - DNA, 25 bp, synthetic construct
  misc_feature 1..25: note = Antisense sequence to target splice site of proprocessed human dystrophin
  source 1..25: mol_type = other DNA
aatttctccttgtttctcaggtaaa

SEQ ID NO: 182 - DNA, 25 bp, synthetic construct
  misc_feature 1..25: note = Antisense sequence to target splice site of proprocessed human dystrophin
  source 1..25: mol_type = other DNA
tttgagcttcaatttctccttgttt

SEQ ID NO: 183 - DNA, 25 bp, synthetic construct
  misc_feature 1..25: note = Antisense sequence to target splice site of proprocessed human dystrophin
  source 1..25: mol_type = other DNA
ttttatttgagcttcaatttctcct

SEQ ID NO: 184 - DNA, 25 bp, synthetic construct
  misc_feature 1..25: note = Antisense sequence to target splice site of proprocessed human dystrophin
  source 1..25: mol_type = other DNA
aagctgcccaaggtcttttatttga

SEQ ID NO: 185 - DNA, 25 bp, synthetic construct
  misc_feature 1..25: note = Antisense sequence to target splice site of proprocessed human dystrophin
  source 1..25: mol_type = other DNA
aggtcttcaagctttttttcaagct

SEQ ID NO: 186 - DNA, 25 bp, synthetic construct
  misc_feature 1..25: note = Antisense sequence to target splice site of proprocessed human dystrophin
  source 1..25: mol_type = other DNA
ttcaagctttttttcaagctgccca

SEQ ID NO: 187 - DNA, 25 bp, synthetic construct
  misc_feature 1..25: note = Antisense sequence to target splice site of proprocessed human dystrophin
  source 1..25: mol_type = other DNA
gatgatttaactgctcttcaaggtc

SEQ ID NO: 188 - DNA, 25 bp, synthetic construct
  misc_feature 1..25: note = Antisense sequence to target splice site of proprocessed human dystrophin
  source 1..25: mol_type = other DNA
ctgctcttcaaggtcttcaagcttt

SEQ ID NO: 189 - DNA, 25 bp, synthetic construct
  misc_feature 1..25: note = Antisense sequence to target splice site of proprocessed human dystrophin
  source 1..25: mol_type = other DNA
aggagataaccacagcagcagatga

SEQ ID NO: 190 - DNA, 25 bp, synthetic construct
  misc_feature 1..25: note = Antisense sequence to target splice site of proprocessed human dystrophin
  source 1..25: mol_type = other DNA
cagcagatgatttaactgctcttca

SEQ ID NO: 191 - DNA, 25 bp, synthetic construct
  misc_feature 1..25: note = Antisense sequence to target splice site of proprocessed human dystrophin
  source 1..25: mol_type = other DNA
atttccaactgattcctaataggag

SEQ ID NO: 192 - DNA, 25 bp, synthetic construct
  misc_feature 1..25: note = Antisense sequence to target splice site of proprocessed human dystrophin
  source 1..25: mol_type = other DNA
cttggtttggttggttataaatttc

SEQ ID NO: 193 - DNA, 25 bp, synthetic construct
  misc_feature 1..25: note = Antisense sequence to target splice site of proprocessed human dystrophin
  source 1..25: mol_type = other DNA
caactgattcctaataggagataac

SEQ ID NO: 194 - DNA, 25 bp, synthetic construct
  misc_feature 1..25: note = Antisense sequence to target splice site of proprocessed human dystrophin
  source 1..25: mol_type = other DNA
cttaacgtcaaatggtccttcttgg

SEQ ID NO: 195 - DNA, 25 bp, synthetic construct
  misc_feature 1..25: note = Antisense sequence to target splice site of proprocessed human dystrophin
  source 1..25: mol_type = other DNA
ttggttataaatttccaactgattc

SEQ ID NO: 196 - DNA, 25 bp, synthetic construct
  misc_feature 1..25: note = Antisense sequence to target splice site of proprocessed human dystrophin
  source 1..25: mol_type = other DNA
cctaccttaacgtcaaatggtcctt

SEQ ID NO: 197 - DNA, 25 bp, synthetic construct
  misc_feature 1..25: note = Antisense sequence to target splice site of proprocessed human dystrophin
  source 1..25: mol_type = other DNA
tccttcttggtttggttggttataa

SEQ ID NO: 198 - DNA, 25 bp, synthetic construct
  misc_feature 1..25: note = Antisense sequence to target splice site of proprocessed human dystrophin
  source 1..25: mol_type = other DNA
agttccctaccttaacgtcaaatgg

SEQ ID NO: 199 - DNA, 25 bp, synthetic construct
  misc_feature 1..25: note = Antisense sequence to target splice site of proprocessed human dystrophin
  source 1..25: mol_type = other DNA
caaaaagttccctaccttaacgtca

SEQ ID NO: 200 - DNA, 25 bp, synthetic construct
  misc_feature 1..25: note = Antisense sequence to target splice site of proprocessed human dystrophin
  source 1..25: mol_type = other DNA
taaagcaaaaagttccctaccttaa

SEQ ID NO: 201 - DNA, 25 bp, synthetic construct
  misc_feature 1..25: note = Antisense sequence to target splice site of proprocessed human dystrophin
  source 1..25: mol_type = other DNA
atatttaaagcaaaaagttccctac

SEQ ID NO: 202 - DNA, 20 bp, synthetic construct
  misc_feature 1..20: note = Antisense sequence to target splice site of proprocessed human dystrophin
  source 1..20: mol_type = other DNA
aggaaaatacattttaaaaa

SEQ ID NO: 203 - DNA, 20 bp, synthetic construct
  misc_feature 1..20: note = Antisense sequence to target splice site of proprocessed human dystrophin
  source 1..20: mol_type = other DNA
aaggaaaatacattttaaaa

SEQ ID NO: 204 - DNA, 20 bp, synthetic construct
  misc_feature 1..20: note = Antisense sequence to target splice site of proprocessed human dystrophin
  source 1..20: mol_type = other DNA
cctgaaaggaaaatacattt

SEQ ID NO: 205 - DNA, 20 bp, synthetic construct
  misc_feature 1..20: note = Antisense sequence to target splice site of proprocessed human dystrophin
  source 1..20: mol_type = other DNA
ggaaacctgaaaggaaaata

SEQ ID NO: 206 - DNA, 20 bp, synthetic construct
  misc_feature 1..20: note = Antisense sequence to target splice site of proprocessed human dystrophin
  source 1..20: mol_type = other DNA
gctctggaaacctgaaagga

SEQ ID NO: 207 - DNA, 20 bp, synthetic construct
  misc_feature 1..20: note = Antisense sequence to target splice site of proprocessed human dystrophin
  source 1..20: mol_type = other DNA
gtaaagctctggaaacctga

SEQ ID NO: 208 - DNA, 20 bp, synthetic construct
  misc_feature 1..20: note = Antisense sequence to target splice site of proprocessed human dystrophin
  source 1..20: mol_type = other DNA
ctcaggtaaagctctggaaa

SEQ ID NO: 209 - DNA, 20 bp, synthetic construct
  misc_feature 1..20: note = Antisense sequence to target splice site of proprocessed human dystrophin
  source 1..20: mol_type = other DNA
aatttctccttgtttctcag

SEQ ID NO: 210 - DNA, 20 bp, synthetic construct
  misc_feature 1..20: note = Antisense sequence to target splice site of proprocessed human dystrophin
  source 1..20: mol_type = other DNA
ttttatttgagcttcaattt

SEQ ID NO: 211 - DNA, 20 bp, synthetic construct
  misc_feature 1..20: note = Antisense sequence to target splice site of proprocessed human dystrophin
  source 1..20: mol_type = other DNA
aagctgcccaaggtctttta

SEQ ID NO: 212 - DNA, 20 bp, synthetic construct
  misc_feature 1..20: note = Antisense sequence to target splice site of proprocessed human dystrophin
  source 1..20: mol_type = other DNA
ttcaagctttttttcaagct

SEQ ID NO: 213 - DNA, 20 bp, synthetic construct
  misc_feature 1..20: note = Antisense sequence to target splice site of proprocessed human dystrophin
  source 1..20: mol_type = other DNA
ctgctcttcaaggtcttcaa

SEQ ID NO: 214 - DNA, 20 bp, synthetic construct
  misc_feature 1..20: note = Antisense sequence to target splice site of proprocessed human dystrophin
  source 1..20: mol_type = other DNA
cagcagatgatttaactgct

SEQ ID NO: 215 - DNA, 20 bp, synthetic construct
  misc_feature 1..20: note = Antisense sequence to target splice site of proprocessed human dystrophin
  source 1..20: mol_type = other DNA
aggagataaccacagcagca

SEQ ID NO: 216 - DNA, 20 bp, synthetic construct
  misc_feature 1..20: note = Antisense sequence to target splice site of proprocessed human dystrophin
  source 1..20: mol_type = other DNA
caactgattcctaataggag

SEQ ID NO: 217 - DNA, 20 bp, synthetic construct
  misc_feature 1..20: note = Antisense sequence to target splice site of proprocessed human dystrophin
  source 1..20: mol_type = other DNA
ttggttataaatttccaact

SEQ ID NO: 218 - DNA, 20 bp, synthetic construct
  misc_feature 1..20: note = Antisense sequence to target splice site of proprocessed human dystrophin
  source 1..20: mol_type = other DNA
tccttcttggtttggttggt

SEQ ID NO: 219 - DNA, 20 bp, synthetic construct
  misc_feature 1..20: note = Antisense sequence to target splice site of proprocessed human dystrophin
  source 1..20: mol_type = other DNA
cttaacgtcaaatggtcctt

SEQ ID NO: 220 - DNA, 20 bp, synthetic construct
  misc_feature 1..20: note = Antisense sequence to target splice site of proprocessed human dystrophin
  source 1..20: mol_type = other DNA
cctaccttaacgtcaaatgg

SEQ ID NO: 221 - DNA, 20 bp, synthetic construct
  misc_feature 1..20: note = Antisense sequence to target splice site of proprocessed human dystrophin
  source 1..20: mol_type = other DNA
agttccctaccttaacgtca

SEQ ID NO: 222 - DNA, 20 bp, synthetic construct
  misc_feature 1..20: note = Antisense sequence to target splice site of proprocessed human dystrophin
  source 1..20: mol_type = other DNA
caaaaagttccctaccttaa

SEQ ID NO: 223 - DNA, 20 bp, synthetic construct
  misc_feature 1..20: note = Antisense sequence to target splice site of proprocessed human dystrophin
  source 1..20: mol_type = other DNA
taaagcaaaaagttccctac

SEQ ID NO: 224 - DNA, 20 bp, synthetic construct
  misc_feature 1..20: note = Antisense sequence to target splice site of proprocessed human dystrophin
  source 1..20: mol_type = other DNA
atatttaaagcaaaaagttc

SEQ ID NO: 225 - DNA, 25 bp, synthetic construct
  misc_feature 1..25: note = Antisense sequence to target splice site of proprocessed human dystrophin
  source 1..25: mol_type = other DNA
ctggggaaaagaacccatatagtgc

SEQ ID NO: 226 - DNA, 25 bp, synthetic construct
  misc_feature 1..25: note = Antisense sequence to target splice site of proprocessed human dystrophin
  source 1..25: mol_type = other DNA
tcctggggaaaagaacccatatagt

SEQ ID NO: 227 - DNA, 25 bp, synthetic construct
  misc_feature 1..25: note = Antisense sequence to target splice site of proprocessed human dystrophin
  source 1..25: mol_type = other DNA
gtttcctggggaaaagaacccatat

SEQ ID NO: 228 - DNA, 25 bp, synthetic construct
  misc_feature 1..25: note = Antisense sequence to target splice site of proprocessed human dystrophin
  source 1..25: mol_type = other DNA
cagtttcctggggaaaagaacccat

SEQ ID NO: 229 - DNA, 25 bp, synthetic construct
  misc_feature 1..25: note = Antisense sequence to target splice site of proprocessed human dystrophin
  source 1..25: mol_type = other DNA
tttcagtttcctggggaaaagaacc

SEQ ID NO: 230 - DNA, 25 bp, synthetic construct
  misc_feature 1..25: note = Antisense sequence to target splice site of proprocessed human dystrophin
  source 1..25: mol_type = other DNA
tatttcagtttcctggggaaaagaa

SEQ ID NO: 231 - DNA, 25 bp, synthetic construct
  misc_feature 1..25: note = Antisense sequence to target splice site of proprocessed human dystrophin
  source 1..25: mol_type = other DNA
tgctatttcagtttcctggggaaaa

SEQ ID NO: 232 - DNA, 25 bp, synthetic construct
  misc_feature 1..25: note = Antisense sequence to target splice site of proprocessed human dystrophin
  source 1..25: mol_type = other DNA
actgctatttcagtttcctggggaa

SEQ ID NO: 233 - DNA, 25 bp, synthetic construct
  misc_feature 1..25: note = Antisense sequence to target splice site of proprocessed human dystrophin
  source 1..25: mol_type = other DNA
tgaactgctatttcagtttcctggg

SEQ ID NO: 234 - DNA, 25 bp, synthetic construct
  misc_feature 1..25: note = Antisense sequence to target splice site of proprocessed human dystrophin
  source 1..25: mol_type = other DNA
cttgaactgctatttcagtttcctg

SEQ ID NO: 235 - DNA, 25 bp, synthetic construct
  misc_feature 1..25: note = Antisense sequence to target splice site of proprocessed human dystrophin
  source 1..25: mol_type = other DNA
tagcttgaactgctatttcagtttc

SEQ ID NO: 236 - DNA, 25 bp, synthetic construct
  misc_feature 1..25: note = Antisense sequence to target splice site of proprocessed human dystrophin
  source 1..25: mol_type = other DNA
tttagcttgaactgctatttcagtt

SEQ ID NO: 237 - DNA, 25 bp, synthetic construct
  misc_feature 1..25: note = Antisense sequence to target splice site of proprocessed human dystrophin
  source 1..25: mol_type = other DNA
ttccacatccggttgtttagcttga

SEQ ID NO: 238 - DNA, 25 bp, synthetic construct
  misc_feature 1..25: note = Antisense sequence to target splice site of proprocessed human dystrophin
  source 1..25: mol_type = other DNA
tgccctttagacaaaatctcttcca

SEQ ID NO: 239 - DNA, 25 bp, synthetic construct
  misc_feature 1..25: note = Antisense sequence to target splice site of proprocessed human dystrophin
  source 1..25: mol_type = other DNA
tttagacaaaatctcttccacatcc

SEQ ID NO: 240 - DNA, 25 bp, synthetic construct
  misc_feature 1..25: note = Antisense sequence to target splice site of proprocessed human dystrophin
  source 1..25: mol_type = other DNA
gtttttccttgtacaaatgctgccc

SEQ ID NO: 241 - DNA, 25 bp, synthetic construct
  misc_feature 1..25: note = Antisense sequence to target splice site of proprocessed human dystrophin
  source 1..25: mol_type = other DNA
gtacaaatgctgccctttagacaaa

SEQ ID NO: 242 - DNA, 25 bp, synthetic construct
  misc_feature 1..25: note = Antisense sequence to target splice site of proprocessed human dystrophin
  source 1..25: mol_type = other DNA
cttcactggctgagtggctggtttt

SEQ ID NO: 243 - DNA, 25 bp, synthetic construct
  misc_feature 1..25: note = Antisense sequence to target splice site of proprocessed human dystrophin
  source 1..25: mol_type = other DNA
ggctggtttttccttgtacaaatgc

SEQ ID NO: 244 - DNA, 25 bp, synthetic construct
  misc_feature 1..25: note = Antisense sequence to target splice site of proprocessed human dystrophin
  source 1..25: mol_type = other DNA
attaccttcactggctgagtggctg

SEQ ID NO: 245 - DNA, 25 bp, synthetic construct
  misc_feature 1..25: note = Antisense sequence to target splice site of proprocessed human dystrophin
  source 1..25: mol_type = other DNA
gcttcattaccttcactggctgagt

SEQ ID NO: 246 - DNA, 25 bp, synthetic construct
  misc_feature 1..25: note = Antisense sequence to target splice site of proprocessed human dystrophin
  source 1..25: mol_type = other DNA
aggttgcttcattaccttcactggc

SEQ ID NO: 247 - DNA, 25 bp, synthetic construct
  misc_feature 1..25: note = Antisense sequence to target splice site of proprocessed human dystrophin
  source 1..25: mol_type = other DNA
gctagaggttgcttcattaccttca

SEQ ID NO: 248 - DNA, 25 bp, synthetic construct
  misc_feature 1..25: note = Antisense sequence to target splice site of proprocessed human dystrophin
  source 1..25: mol_type = other DNA
atattgctagaggttgcttcattac

SEQ ID NO: 249 - DNA, 20 bp, synthetic construct
  misc_feature 1..20: note = Antisense sequence to target splice site of proprocessed human dystrophin
  source 1..20: mol_type = other DNA
gaaaagaacccatatagtgc

SEQ ID NO: 250 - DNA, 20 bp, synthetic construct
  misc_feature 1..20: note = Antisense sequence to target splice site of proprocessed human dystrophin
  source 1..20: mol_type = other DNA
gggaaaagaacccatatagt

SEQ ID NO: 251 - DNA, 20 bp, synthetic construct
  misc_feature 1..20: note = Antisense sequence to target splice site of proprocessed human dystrophin
  source 1..20: mol_type = other DNA
tcctggggaaaagaacccat

SEQ ID NO: 252 - DNA, 20 bp, synthetic construct
  misc_feature 1..20: note = Antisense sequence to target splice site of proprocessed human dystrophin
  source 1..20: mol_type = other DNA
cagtttcctggggaaaagaa

SEQ ID NO: 253 - DNA, 20 bp, synthetic construct
  misc_feature 1..20: note = Antisense sequence to target splice site of proprocessed human dystrophin
  source 1..20: mol_type = other DNA
tatttcagtttcctggggaa

SEQ ID NO: 254 - DNA, 20 bp, synthetic construct
  misc_feature 1..20: note = Antisense sequence to target splice site of proprocessed human dystrophin
  source 1..20: mol_type = other DNA
actgctatttcagtttcctg

SEQ ID NO: 255 - DNA, 20 bp, synthetic construct
  misc_feature 1..20: note = Antisense sequence to target splice site of proprocessed human dystrophin
  source 1..20: mol_type = other DNA
cttgaactgctatttcagtt

SEQ ID NO: 256 - DNA, 20 bp, synthetic construct
  misc_feature 1..20: note = Antisense sequence to target splice site of proprocessed human dystrophin
  source 1..20: mol_type = other DNA
tttagcttgaactgctattt

SEQ ID NO: 257 - DNA, 20 bp, synthetic construct
  misc_feature 1..20: note = Antisense sequence to target splice site of proprocessed human dystrophin
  source 1..20: mol_type = other DNA
ttccacatccggttgtttag

SEQ ID NO: 258 - DNA, 20 bp, synthetic construct
  misc_feature 1..20: note = Antisense sequence to target splice site of proprocessed human dystrophin
  source 1..20: mol_type = other DNA
tttagacaaaatctcttcca

SEQ ID NO: 259 - DNA, 20 bp, synthetic construct
  misc_feature 1..20: note = Antisense sequence to target splice site of proprocessed human dystrophin
  source 1..20: mol_type = other DNA
gtacaaatgctgccctttag

SEQ ID NO: 260 - DNA, 20 bp, synthetic construct
  misc_feature 1..20: note = Antisense sequence to target splice site of proprocessed human dystrophin
  source 1..20: mol_type = other DNA
ggctggtttttccttgtaca

SEQ ID NO: 261 - DNA, 20 bp, synthetic construct
  misc_feature 1..20: note = Antisense sequence to target splice site of proprocessed human dystrophin
  source 1..20: mol_type = other DNA
cttcactggctgagtggctg

SEQ ID NO: 262 - DNA, 20 bp, synthetic construct
  misc_feature 1..20: note = Antisense sequence to target splice site of proprocessed human dystrophin
  source 1..20: mol_type = other DNA
attaccttcactggctgagt

SEQ ID NO: 263 - DNA, 20 bp, synthetic construct
  misc_feature 1..20: note = Antisense sequence to target splice site of proprocessed human dystrophin
  source 1..20: mol_type = other DNA
gcttcattaccttcactggc

SEQ ID NO: 264 - DNA, 20 bp, synthetic construct
  misc_feature 1..20: note = Antisense sequence to target splice site of proprocessed human dystrophin
  source 1..20: mol_type = other DNA
aggttgcttcattaccttca

SEQ ID NO: 265 - DNA, 20 bp, synthetic construct
  misc_feature 1..20: note = Antisense sequence to target splice site of proprocessed human dystrophin
  source 1..20: mol_type = other DNA
gctagaggttgcttcattac

SEQ ID NO: 266 - DNA, 20 bp, synthetic construct
  misc_feature 1..20: note = Antisense sequence to target splice site of proprocessed human dystrophin
  source 1..20: mol_type = other DNA
atattgctagaggttgcttc

SEQ ID NO: 267 - DNA, 25 bp, synthetic construct
  misc_feature 1..25: note = Antisense sequence to target splice site of proprocessed human dystrophin
  source 1..25: mol_type = other DNA
ctttaacagaaaagcatacacatta

SEQ ID NO: 268 - DNA, 25 bp, synthetic construct
  misc_feature 1..25: note = Antisense sequence to target splice site of proprocessed human dystrophin
  source 1..25: mol_type = other DNA
tcctctttaacagaaaagcatacac

SEQ ID NO: 269 - DNA, 25 bp, synthetic construct
  misc_feature 1..25: note = Antisense sequence to target splice site of proprocessed human dystrophin
  source 1..25: mol_type = other DNA
ttcctctttaacagaaaagcataca

SEQ ID NO: 270 - DNA, 25 bp, synthetic construct
  misc_feature 1..25: note = Antisense sequence to target splice site of proprocessed human dystrophin
  source 1..25: mol_type = other DNA
taacttcctctttaacagaaaagca

SEQ ID NO: 271 - DNA, 25 bp, synthetic construct
  misc_feature 1..25: note = Antisense sequence to target splice site of proprocessed human dystrophin
  source 1..25: mol_type = other DNA
ctaacttcctctttaacagaaaagc

SEQ ID NO: 272 - DNA, 25 bp, synthetic construct
  misc_feature 1..25: note = Antisense sequence to target splice site of proprocessed human dystrophin
  source 1..25: mol_type = other DNA
tcttctaacttcctctttaacagaa

SEQ ID NO: 273 - DNA, 25 bp, synthetic construct
  misc_feature 1..25: note = Antisense sequence to target splice site of proprocessed human dystrophin
  source 1..25: mol_type = other DNA
atcttctaacttcctctttaacaga

SEQ ID NO: 274 - DNA, 25 bp, synthetic construct
  misc_feature 1..25: note = Antisense sequence to target splice site of proprocessed human dystrophin
  source 1..25: mol_type = other DNA
tcagatcttctaacttcctctttaa

SEQ ID NO: 275 - DNA, 25 bp, synthetic construct
  misc_feature 1..25: note = Antisense sequence to target splice site of proprocessed human dystrophin
  source 1..25: mol_type = other DNA
ctcagatcttctaacttcctcttta

SEQ ID NO: 276 - DNA, 25 bp, synthetic construct
  misc_feature 1..25: note = Antisense sequence to target splice site of proprocessed human dystrophin
  source 1..25: mol_type = other DNA
agagctcagatcttctaacttcctc

SEQ ID NO: 277 - DNA, 25 bp, synthetic construct
  misc_feature 1..25: note = Antisense sequence to target splice site of proprocessed human dystrophin
  source 1..25: mol_type = other DNA
cagagctcagatcttctaacttcct

SEQ ID NO: 278 - DNA, 24 bp, synthetic construct
  misc_feature 1..24: note = Antisense sequence to target splice site of proprocessed human dystrophin
  source 1..24: mol_type = other DNA
cactcagagctcagatcttctact

SEQ ID NO: 279 - DNA, 25 bp, synthetic construct
  misc_feature 1..25: note = Antisense sequence to target splice site of proprocessed human dystrophin
  source 1..25: mol_type = other DNA
ccttccactcagagctcagatcttc

SEQ ID NO: 280 - DNA, 25 bp, synthetic construct
  misc_feature 1..25: note = Antisense sequence to target splice site of proprocessed human dystrophin
  source 1..25: mol_type = other DNA
gtaaacggtttaccgccttccactc

SEQ ID NO: 281 - DNA, 25 bp, synthetic construct
  misc_feature 1..25: note = Antisense sequence to target splice site of proprocessed human dystrophin
  source 1..25: mol_type = other DNA
ctttgccctcagctcttgaagtaaa

SEQ ID NO: 282 - DNA, 25 bp, synthetic construct
  misc_feature 1..25: note = Antisense sequence to target splice site of proprocessed human dystrophin
  source 1..25: mol_type = other DNA
ccctcagctcttgaagtaaacggtt

SEQ ID NO: 283 - DNA, 25 bp, synthetic construct
  misc_feature 1..25: note = Antisense sequence to target splice site of proprocessed human dystrophin
  source 1..25: mol_type = other DNA
ccaggagctaggtcaggctgctttg

SEQ ID NO: 284 - DNA, 25 bp, synthetic construct
  misc_feature 1..25: note = Antisense sequence to target splice site of proprocessed human dystrophin
  source 1..25: mol_type = other DNA
ggtcaggctgctttgccctcagctc

SEQ ID NO: 285 - DNA, 25 bp, synthetic construct
  misc_feature 1..25: note = Antisense sequence to target splice site of proprocessed human dystrophin
  source 1..25: mol_type = other DNA
aggctccaatagtggtcagtccagg

SEQ ID NO: 286 - DNA, 25 bp, synthetic construct
  misc_feature 1..25: note = Antisense sequence to target splice site of proprocessed human dystrophin
  source 1..25: mol_type = other DNA
tcagtccaggagctaggtcaggctg

SEQ ID NO: 287 - DNA, 25 bp, synthetic construct
  misc_feature 1..25: note = Antisense sequence to target splice site of proprocessed human dystrophin
  source 1..25: mol_type = other DNA
cttacaggctccaatagtggtcagt

SEQ ID NO: 288 - DNA, 25 bp, synthetic construct
  misc_feature 1..25: note = Antisense sequence to target splice site of proprocessed human dystrophin
  source 1..25: mol_type = other DNA
gtatacttacaggctccaatagtgg

SEQ ID NO: 289 - DNA, 25 bp, synthetic construct
  misc_feature 1..25: note = Antisense sequence to target splice site of proprocessed human dystrophin
  source 1..25: mol_type = other DNA
atccagtatacttacaggctccaat

SEQ ID NO: 290 - DNA, 25 bp, synthetic construct
  misc_feature 1..25: note = Antisense sequence to target splice site of proprocessed human dystrophin
  source 1..25: mol_type = other DNA
atgggatccagtatacttacaggct

SEQ ID NO: 291 - DNA, 25 bp, synthetic construct
  misc_feature 1..25: note = Antisense sequence to target splice site of proprocessed human dystrophin
  source 1..25: mol_type = other DNA
agagaatgggatccagtatacttac

SEQ ID NO: 292 - DNA, 20 bp, synthetic construct
  misc_feature 1..20: note = Antisense sequence to target splice site of proprocessed human dystrophin
  source 1..20: mol_type = other DNA
acagaaaagcatacacatta

SEQ ID NO: 293 - DNA, 20 bp, synthetic construct
  misc_feature 1..20: note = Antisense sequence to target splice site of proprocessed human dystrophin
  source 1..20: mol_type = other DNA
tttaacagaaaagcatacac

SEQ ID NO: 294 - DNA, 20 bp, synthetic construct
  misc_feature 1..20: note = Antisense sequence to target splice site of proprocessed human dystrophin
  source 1..20: mol_type = other DNA
tcctctttaacagaaaagca

SEQ ID NO: 295 - DNA, 20 bp, synthetic construct
  misc_feature 1..20: note = Antisense sequence to target splice site of proprocessed human dystrophin
  source 1..20: mol_type = other DNA
taacttcctctttaacagaa

SEQ ID NO: 296 - DNA, 20 bp, synthetic construct
  misc_feature 1..20: note = Antisense sequence to target splice site of proprocessed human dystrophin
  source 1..20: mol_type = other DNA
tcttctaacttcctctttaa

SEQ ID NO: 297 - DNA, 20 bp, synthetic construct
  misc_feature 1..20: note = Antisense sequence to target splice site of proprocessed human dystrophin
  source 1..20: mol_type = other DNA
tcagatcttctaacttcctc

SEQ ID NO: 298 - DNA, 20 bp, synthetic construct
  misc_feature 1..20: note = Antisense sequence to target splice site of proprocessed human dystrophin
  source 1..20: mol_type = other DNA
ccttccactcagagctcaga

SEQ ID NO: 299 - DNA, 20 bp, synthetic construct
  misc_feature 1..20: note = Antisense sequence to target splice site of proprocessed human dystrophin
  source 1..20: mol_type = other DNA
gtaaacggtttaccgccttc

SEQ ID NO: 300 - DNA, 20 bp, synthetic construct
  misc_feature 1..20: note = Antisense sequence to target splice site of proprocessed human dystrophin
  source 1..20: mol_type = other DNA
ccctcagctcttgaagtaaa

SEQ ID NO: 301 - DNA, 20 bp, synthetic construct
  misc_feature 1..20: note = Antisense sequence to target splice site of proprocessed human dystrophin
  source 1..20: mol_type = other DNA
ggtcaggctgctttgccctc

SEQ ID NO: 302 - DNA, 20 bp, synthetic construct
  misc_feature 1..20: note = Antisense sequence to target splice site of proprocessed human dystrophin
  source 1..20: mol_type = other DNA
tcagtccaggagctaggtca

SEQ ID NO: 303 - DNA, 20 bp, synthetic construct
  misc_feature 1..20: note = Antisense sequence to target splice site of proprocessed human dystrophin
  source 1..20: mol_type = other DNA
aggctccaatagtggtcagt

SEQ ID NO: 304 - DNA, 20 bp, synthetic construct
  misc_feature 1..20: note = Antisense sequence to target splice site of proprocessed human dystrophin
  source 1..20: mol_type = other DNA
cttacaggctccaatagtgg

SEQ ID NO: 305 - DNA, 20 bp, synthetic construct
  misc_feature 1..20: note = Antisense sequence to target splice site of proprocessed human dystrophin
  source 1..20: mol_type = other DNA
gtatacttacaggctccaat

SEQ ID NO: 306 - DNA, 20 bp, synthetic construct
  misc_feature 1..20: note = Antisense sequence to target splice site of proprocessed human dystrophin
  source 1..20: mol_type = other DNA
atccagtatacttacaggct

SEQ ID NO: 307 - DNA, 20 bp, synthetic construct
  misc_feature 1..20: note = Antisense sequence to target splice site of proprocessed human dystrophin
  source 1..20: mol_type = other DNA
atgggatccagtatacttac

SEQ ID NO: 308 - DNA, 20 bp, synthetic construct
  misc_feature 1..20: note = Antisense sequence to target splice site of proprocessed human dystrophin
  source 1..20: mol_type = other DNA
agagaatgggatccagtata

SEQ ID NO: 309 - DNA, 26 bp, synthetic construct
  misc_feature 1..26: note = Antisense sequence to target splice site of proprocessed human dystrophin
  source 1..26: mol_type = other DNA
ctaaaatattttgggtttttgcaaaa

SEQ ID NO: 310 - DNA, 26 bp, synthetic construct
  misc_feature 1..26: note = Antisense sequence to target splice site of proprocessed human dystrophin
  source 1..26: mol_type = other DNA
gctaaaatattttgggtttttgcaaa

SEQ ID NO: 311 - DNA, 26 bp, synthetic construct
  misc_feature 1..26: note = Antisense sequence to target splice site of proprocessed human dystrophin
  source 1..26: mol_type = other DNA
taggagctaaaatattttgggttttt

SEQ ID NO: 312 - DNA, 25 bp, synthetic construct
  misc_feature 1..25: note = Antisense sequence to target splice site of proprocessed human dystrophin
  source 1..25: mol_type = other DNA
agtaggagctaaaatattttgggtt

SEQ ID NO: 313 - DNA, 25 bp, synthetic construct
  misc_feature 1..25: note = Antisense sequence to target splice site of proprocessed human dystrophin
  source 1..25: mol_type = other DNA
tgagtaggagctaaaatattttggg

SEQ ID NO: 314 - DNA, 26 bp, synthetic construct
  misc_feature 1..26: note = Antisense sequence to target splice site of proprocessed human dystrophin
  source 1..26: mol_type = other DNA
ctgagtaggagctaaaatattttggg

SEQ ID NO: 315 - DNA, 25 bp, synthetic construct
  misc_feature 1..25: note = Antisense sequence to target splice site of proprocessed human dystrophin
  source 1..25: mol_type = other DNA
cagtctgagtaggagctaaaatatt

SEQ ID NO: 316 - DNA, 26 bp, synthetic construct
  misc_feature 1..26: note = Antisense sequence to target splice site of proprocessed human dystrophin
  source 1..26: mol_type = other DNA
acagtctgagtaggagctaaaatatt

SEQ ID NO: 317 - DNA, 26 bp, synthetic construct
  misc_feature 1..26: note = Antisense sequence to target splice site of proprocessed human dystrophin
  source 1..26: mol_type = other DNA
gagtaacagtctgagtaggagctaaa

SEQ ID NO: 318 - DNA, 25 bp, synthetic construct
  misc_feature 1..25: note = Antisense sequence to target splice site of proprocessed human dystrophin
  source 1..25: mol_type = other DNA
cagagtaacagtctgagtaggagct

SEQ ID NO: 319 - DNA, 26 bp, synthetic construct
  misc_feature 1..26: note = Antisense sequence to target splice site of proprocessed human dystrophin
  source 1..26: mol_type = other DNA
caccagagtaacagtctgagtaggag

SEQ ID NO: 320 - DNA, 25 bp, synthetic construct
  misc_feature 1..25: note = Antisense sequence to target splice site of proprocessed human dystrophin
  source 1..25: mol_type = other DNA
gtcaccagagtaacagtctgagtag

SEQ ID NO: 321 - DNA, 26 bp, synthetic construct
  misc_feature 1..26: note = Antisense sequence to target splice site of proprocessed human dystrophin
  source 1..26: mol_type = other DNA
aaccacaggttgtgtcaccagagtaa

SEQ ID NO: 322 - DNA, 25 bp, synthetic construct
  misc_feature 1..25: note = Antisense sequence to target splice site of proprocessed human dystrophin
  source 1..25: mol_type = other DNA
gttgtgtcaccagagtaacagtctg

SEQ ID NO: 323 - DNA, 26 bp, synthetic construct
  misc_feature 1..26: note = Antisense sequence to target splice site of proprocessed human dystrophin
  source 1..26: mol_type = other DNA
tggcagtttccttagtaaccacaggt

SEQ ID NO: 324 - DNA, 26 bp, synthetic construct
  misc_feature 1..26: note = Antisense sequence to target splice site of proprocessed human dystrophin
  source 1..26: mol_type = other DNA
atttctagtttggagatggcagtttc

SEQ ID NO: 325 - DNA, 25 bp, synthetic construct
  misc_feature 1..25: note = Antisense sequence to target splice site of proprocessed human dystrophin
  source 1..25: mol_type = other DNA
ggaagatggcatttctagtttggag

SEQ ID NO: 326 - DNA, 26 bp, synthetic construct
  misc_feature 1..26: note = Antisense sequence to target splice site of proprocessed human dystrophin
  source 1..26: mol_type = other DNA
catcaaggaagatggcatttctagtt

SEQ ID NO: 327 - DNA, 26 bp, synthetic construct
  misc_feature 1..26: note = Antisense sequence to target splice site of proprocessed human dystrophin
  source 1..26: mol_type = other DNA
gagcaggtacctccaacatcaaggaa

SEQ ID NO: 328 - DNA, 25 bp, synthetic construct
  misc_feature 1..25: note = Antisense sequence to target splice site of proprocessed human dystrophin
  source 1..25: mol_type = other DNA
atctgccagagcaggtacctccaac

SEQ ID NO: 329 - DNA, 26 bp, synthetic construct
  misc_feature 1..26: note = Antisense sequence to target splice site of proprocessed human dystrophin
  source 1..26: mol_type = other DNA
aagttctgtccaagcccggttgaaat

SEQ ID NO: 330 - DNA, 26 bp, synthetic construct
  misc_feature 1..26: note = Antisense sequence to target splice site of proprocessed human dystrophin
  source 1..26: mol_type = other DNA
cggttgaaatctgccagagcaggtac

SEQ ID NO: 331 - DNA, 26 bp, synthetic construct
  misc_feature 1..26: note = Antisense sequence to target splice site of proprocessed human dystrophin
  source 1..26: mol_type = other DNA
gagaaagccagtcggtaagttctgtc

SEQ ID NO: 332 - DNA, 25 bp, synthetic construct
  misc_feature 1..25: note = Antisense sequence to target splice site of proprocessed human dystrophin
  source 1..25: mol_type = other DNA
gtcggtaagttctgtccaagcccgg

SEQ ID NO: 333 - DNA, 26 bp, synthetic construct
  misc_feature 1..26: note = Antisense sequence to target splice site of proprocessed human dystrophin
  source 1..26: mol_type = other DNA
ataacttgatcaagcagagaaagcca

SEQ ID NO: 334 - DNA, 25 bp, synthetic construct
  misc_feature 1..25: note = Antisense sequence to target splice site of proprocessed human dystrophin
  source 1..25: mol_type = other DNA
aagcagagaaagccagtcggtaagt

SEQ ID NO: 335 - DNA, 26 bp, synthetic construct
  misc_feature 1..26: note = Antisense sequence to target splice site of proprocessed human dystrophin
  source 1..26: mol_type = other DNA
caccctctgtgattttataacttgat

SEQ ID NO: 336 - DNA, 26 bp, synthetic construct
  misc_feature 1..26: note = Antisense sequence to target splice site of proprocessed human dystrophin
  source 1..26: mol_type = other DNA
caaggtcacccaccatcaccctctgt

SEQ ID NO: 337 - DNA, 25 bp, synthetic construct
  misc_feature 1..25: note = Antisense sequence to target splice site of proprocessed human dystrophin
  source 1..25: mol_type = other DNA
catcaccctctgtgattttataact

SEQ ID NO: 338 - DNA, 26 bp, synthetic construct
  misc_feature 1..26: note = Antisense sequence to target splice site of proprocessed human dystrophin
  source 1..26: mol_type = other DNA
cttctgcttgatgatcatctcgttga

SEQ ID NO: 339 - DNA, 26 bp, synthetic construct
  misc_feature 1..26: note = Antisense sequence to target splice site of proprocessed human dystrophin
  source 1..26: mol_type = other DNA
ccttctgcttgatgatcatctcgttg

SEQ ID NO: 340 - DNA, 26 bp, synthetic construct
  misc_feature 1..26: note = Antisense sequence to target splice site of proprocessed human dystrophin
  source 1..26: mol_type = other DNA
atctcgttgatatcctcaaggtcacc

SEQ ID NO: 341 - DNA, 26 bp, synthetic construct
  misc_feature 1..26: note = Antisense sequence to target splice site of proprocessed human dystrophin
  source 1..26: mol_type = other DNA
tcataccttctgcttgatgatcatct

SEQ ID NO: 342 - DNA, 26 bp, synthetic construct
  misc_feature 1..26: note = Antisense sequence to target splice site of proprocessed human dystrophin
  source 1..26: mol_type = other DNA
tcattttttctcataccttctgcttg

SEQ ID NO: 343 - DNA, 26 bp, synthetic construct
  misc_feature 1..26: note = Antisense sequence to target splice site of proprocessed human dystrophin
  source 1..26: mol_type = other DNA
ttttctcataccttctgcttgatgat

SEQ ID NO: 344 - DNA, 26 bp, synthetic construct
  misc_feature 1..26: note = Antisense sequence to target splice site of proprocessed human dystrophin
  source 1..26: mol_type = other DNA
ttttatcattttttctcataccttct

SEQ ID NO: 345 - DNA, 26 bp, synthetic construct
  misc_feature 1..26: note = Antisense sequence to target splice site of proprocessed human dystrophin
  source 1..26: mol_type = other DNA
ccaacttttatcattttttctcatac

SEQ ID NO: 346 - DNA, 20 bp, synthetic construct
  misc_feature 1..20: note = Antisense sequence to target splice site of proprocessed human dystrophin
  source 1..20: mol_type = other DNA
atattttgggtttttgcaaa

SEQ ID NO: 347 - DNA, 20 bp, synthetic construct
  misc_feature 1..20: note = Antisense sequence to target splice site of proprocessed human dystrophin
  source 1..20: mol_type = other DNA
aaaatattttgggtttttgc

SEQ ID NO: 348 - DNA, 20 bp, synthetic construct
  misc_feature 1..20: note = Antisense sequence to target splice site of proprocessed human dystrophin
  source 1..20: mol_type = other DNA
gagctaaaatattttgggtt

SEQ ID NO: 349 - DNA, 20 bp, synthetic construct
  misc_feature 1..20: note = Antisense sequence to target splice site of proprocessed human dystrophin
  source 1..20: mol_type = other DNA
agtaggagctaaaatatttt

SEQ ID NO: 350 - DNA, 20 bp, synthetic construct
  misc_feature 1..20: note = Antisense sequence to target splice site of proprocessed human dystrophin
  source 1..20: mol_type = other DNA
gtctgagtaggagctaaaat

SEQ ID NO: 351 - DNA, 20 bp, synthetic construct
  misc_feature 1..20: note = Antisense sequence to target splice site of proprocessed human dystrophin
  source 1..20: mol_type = other DNA
taacagtctgagtaggagct

SEQ ID NO: 352 - DNA, 20 bp, synthetic construct
  misc_feature 1..20: note = Antisense sequence to target splice site of proprocessed human dystrophin
  source 1..20: mol_type = other DNA
cagagtaacagtctgagtag

SEQ ID NO: 353 - DNA, 20 bp, synthetic construct
  misc_feature 1..20: note = Antisense sequence to target splice site of proprocessed human dystrophin
  source 1..20: mol_type = other DNA
cacaggttgtgtcaccagag

SEQ ID NO: 354 - DNA, 20 bp, synthetic construct
  misc_feature 1..20: note = Antisense sequence to target splice site of proprocessed human dystrophin
  source 1..20: mol_type = other DNA
agtttccttagtaaccacag

SEQ ID NO: 355 - DNA, 20 bp, synthetic construct
  misc_feature 1..20: note = Antisense sequence to target splice site of proprocessed human dystrophin
  source 1..20: mol_type = other DNA
tagtttggagatggcagttt

SEQ ID NO: 356 - DNA, 20 bp, synthetic construct
  misc_feature 1..20: note = Antisense sequence to target splice site of proprocessed human dystrophin
  source 1..20: mol_type = other DNA
ggaagatggcatttctagtt

SEQ ID NO: 357 - DNA, 20 bp, synthetic construct
  misc_feature 1..20: note = Antisense sequence to target splice site of proprocessed human dystrophin
  source 1..20: mol_type = other DNA
tacctccaacatcaaggaag

SEQ ID NO: 358 - DNA, 20 bp, synthetic construct
  misc_feature 1..20: note = Antisense sequence to target splice site of proprocessed human dystrophin
  source 1..20: mol_type = other DNA
atctgccagagcaggtacct

SEQ ID NO: 359 - DNA, 20 bp, synthetic construct
  misc_feature 1..20: note = Antisense sequence to target splice site of proprocessed human dystrophin
  source 1..20: mol_type = other DNA
ccaagcccggttgaaatctg

SEQ ID NO: 360 - DNA, 20 bp, synthetic construct
  misc_feature 1..20: note = Antisense sequence to target splice site of proprocessed human dystrophin
  source 1..20: mol_type = other DNA
gtcggtaagttctgtccaag

SEQ ID NO: 361 - DNA, 20 bp, synthetic construct
  misc_feature 1..20: note = Antisense sequence to target splice site of proprocessed human dystrophin
  source 1..20: mol_type = other DNA
aagcagagaaagccagtcgg

SEQ ID NO: 362 - DNA, 20 bp, synthetic construct
  misc_feature 1..20: note = Antisense sequence to target splice site of proprocessed human dystrophin
  source 1..20: mol_type = other DNA
ttttataacttgatcaagca

SEQ ID NO: 363 - DNA, 20 bp, synthetic construct
  misc_feature 1..20: note = Antisense sequence to target splice site of proprocessed human dystrophin
  source 1..20: mol_type = other DNA
catcaccctctgtgatttta

SEQ ID NO: 364 - DNA, 20 bp, synthetic construct
  misc_feature 1..20: note = Antisense sequence to target splice site of proprocessed human dystrophin
  source 1..20: mol_type = other DNA
ctcaaggtcacccaccatca

SEQ ID NO: 365 - DNA, 20 bp, synthetic construct
  misc_feature 1..20: note = Antisense sequence to target splice site of proprocessed human dystrophin
  source 1..20: mol_type = other DNA
catctcgttgatatcctcaa

SEQ ID NO: 366 - DNA, 20 bp, synthetic construct
  misc_feature 1..20: note = Antisense sequence to target splice site of proprocessed human dystrophin
  source 1..20: mol_type = other DNA
cttctgcttgatgatcatct

SEQ ID NO: 367 - DNA, 20 bp, synthetic construct
  misc_feature 1..20: note = Antisense sequence to target splice site of proprocessed human dystrophin
  source 1..20: mol_type = other DNA
cataccttctgcttgatgat

SEQ ID NO: 368 - DNA, 20 bp, synthetic construct
  misc_feature 1..20: note = Antisense sequence to target splice site of proprocessed human dystrophin
  source 1..20: mol_type = other DNA
tttctcataccttctgcttg

SEQ ID NO: 369 - DNA, 20 bp, synthetic construct
  misc_feature 1..20: note = Antisense sequence to target splice site of proprocessed human dystrophin
  source 1..20: mol_type = other DNA
cattttttctcataccttct

SEQ ID NO: 370 - DNA, 20 bp, synthetic construct
  misc_feature 1..20: note = Antisense sequence to target splice site of proprocessed human dystrophin
  source 1..20: mol_type = other DNA
tttatcattttttctcatac

SEQ ID NO: 371 - DNA, 20 bp, synthetic construct
  misc_feature 1..20: note = Antisense sequence to target splice site of proprocessed human dystrophin
  source 1..20: mol_type = other DNA
caacttttatcattttttct

SEQ ID NO: 372 - DNA, 25 bp, synthetic construct
  misc_feature 1..25: note = Antisense sequence to target splice site of proprocessed human dystrophin
  source 1..25: mol_type = other DNA
ctgtaagaacaaatatcccttagta

SEQ ID NO: 373 - DNA, 25 bp, synthetic construct
  misc_feature 1..25: note = Antisense sequence to target splice site of proprocessed human dystrophin
  source 1..25: mol_type = other DNA
tgcctgtaagaacaaatatccctta

SEQ ID NO: 374 - DNA, 25 bp, synthetic construct
  misc_feature 1..25: note = Antisense sequence to target splice site of proprocessed human dystrophin
  source 1..25: mol_type = other DNA
gttgcctgtaagaacaaatatccct

SEQ ID NO: 375 - DNA, 25 bp, synthetic construct
  misc_feature 1..25: note = Antisense sequence to target splice site of proprocessed human dystrophin
  source 1..25: mol_type = other DNA
attgttgcctgtaagaacaaatatc

SEQ ID NO: 376 - DNA, 25 bp, synthetic construct
  misc_feature 1..25: note = Antisense sequence to target splice site of proprocessed human dystrophin
  source 1..25: mol_type = other DNA
gcattgttgcctgtaagaacaaata

SEQ ID NO: 377 - DNA, 25 bp, synthetic construct
  misc_feature 1..25: note = Antisense sequence to target splice site of proprocessed human dystrophin
  source 1..25: mol_type = other DNA
cctgcattgttgcctgtaagaacaa

SEQ ID NO: 378 - DNA, 25 bp, synthetic construct
  misc_feature 1..25: note = Antisense sequence to target splice site of proprocessed human dystrophin
  source 1..25: mol_type = other DNA
atcctgcattgttgcctgtaagaac

SEQ ID NO: 379 - DNA, 25 bp, synthetic construct
  misc_feature 1..25: note = Antisense sequence to target splice site of proprocessed human dystrophin
  source 1..25: mol_type = other DNA
caaatcctgcattgttgcctgtaag

SEQ ID NO: 380 - DNA, 25 bp, synthetic construct
  misc_feature 1..25: note = Antisense sequence to target splice site of proprocessed human dystrophin
  source 1..25: mol_type = other DNA
tccaaatcctgcattgttgcctgta

SEQ ID NO: 381 - DNA, 25 bp, synthetic construct
  misc_feature 1..25: note = Antisense sequence to target splice site of proprocessed human dystrophin
  source 1..25: mol_type = other DNA
tgttccaaatcctgcattgttgcct

SEQ ID NO: 382 - DNA, 25 bp, synthetic construct
  misc_feature 1..25: note = Antisense sequence to target splice site of proprocessed human dystrophin
  source 1..25: mol_type = other DNA
tctgttccaaatcctgcattgttgc

SEQ ID NO: 383 - DNA, 25 bp, synthetic construct
  misc_feature 1..25: note = Antisense sequence to target splice site of proprocessed human dystrophin
  source 1..25: mol_type = other DNA
aactggggacgcctctgttccaaat

SEQ ID NO: 384 - DNA, 25 bp, synthetic construct
  misc_feature 1..25: note = Antisense sequence to target splice site of proprocessed human dystrophin
  source 1..25: mol_type = other DNA
gcctctgttccaaatcctgcattgt

SEQ ID NO: 385 - DNA, 25 bp, synthetic construct
  misc_feature 1..25: note = Antisense sequence to target splice site of proprocessed human dystrophin
  source 1..25: mol_type = other DNA
cagcggtaatgagttcttccaactg

SEQ ID NO: 386 - DNA, 25 bp, synthetic construct
  misc_feature 1..25: note = Antisense sequence to target splice site of proprocessed human dystrophin
  source 1..25: mol_type = other DNA
cttccaactggggacgcctctgttc

SEQ ID NO: 387 - DNA, 25 bp, synthetic construct
  misc_feature 1..25: note = Antisense sequence to target splice site of proprocessed human dystrophin
  source 1..25: mol_type = other DNA
cttgtttttcaaattttgggcagcg

SEQ ID NO: 388 - DNA, 25 bp, synthetic construct
  misc_feature 1..25: note = Antisense sequence to target splice site of proprocessed human dystrophin
  source 1..25: mol_type = other DNA
ctagcctcttgattgctggtcttgt

SEQ ID NO: 389 - DNA, 25 bp, synthetic construct
  misc_feature 1..25: note = Antisense sequence to target splice site of proprocessed human dystrophin
  source 1..25: mol_type = other DNA
ttttcaaattttgggcagcggtaat

SEQ ID NO: 390 - DNA, 25 bp, synthetic construct
  misc_feature 1..25: note = Antisense sequence to target splice site of proprocessed human dystrophin
  source 1..25: mol_type = other DNA
ttcgatccgtaatgattgttctagc

SEQ ID NO: 391 - DNA, 25 bp, synthetic construct
  misc_feature 1..25: note = Antisense sequence to target splice site of proprocessed human dystrophin
  source 1..25: mol_type = other DNA
gattgctggtcttgtttttcaaatt

SEQ ID NO: 392 - DNA, 25 bp, synthetic construct
  misc_feature 1..25: note = Antisense sequence to target splice site of proprocessed human dystrophin
  source 1..25: mol_type = other DNA
cttacttcgatccgtaatgattgtt

SEQ ID NO: 393 - DNA, 25 bp, synthetic construct
  misc_feature 1..25: note = Antisense sequence to target splice site of proprocessed human dystrophin
  source 1..25: mol_type = other DNA
ttgttctagcctcttgattgctggt

SEQ ID NO: 394 - DNA, 25 bp, synthetic construct
  misc_feature 1..25: note = Antisense sequence to target splice site of proprocessed human dystrophin
  source 1..25: mol_type = other DNA
aaaaacttacttcgatccgtaatga

SEQ ID NO: 395 - DNA, 25 bp, synthetic construct
  misc_feature 1..25: note = Antisense sequence to target splice site of proprocessed human dystrophin
  source 1..25: mol_type = other DNA
tgttaaaaaacttacttcgatccgt

SEQ ID NO: 396 - DNA, 25 bp, synthetic construct
  misc_feature 1..25: note = Antisense sequence to target splice site of proprocessed human dystrophin
  source 1..25: mol_type = other DNA
atgcttgttaaaaaacttacttcga

SEQ ID NO: 397 - DNA, 25 bp, synthetic construct
  misc_feature 1..25: note = Antisense sequence to target splice site of proprocessed human dystrophin
  source 1..25: mol_type = other DNA
gtcccatgcttgttaaaaaacttac

SEQ ID NO: 398 - DNA, 20 bp, synthetic construct
  misc_feature 1..20: note = Antisense sequence to target splice site of proprocessed human dystrophin
  source 1..20: mol_type = other DNA
agaacaaatatcccttagta

SEQ ID NO: 399 - DNA, 20 bp, synthetic construct
  misc_feature 1..20: note = Antisense sequence to target splice site of proprocessed human dystrophin
  source 1..20: mol_type = other DNA
gtaagaacaaatatccctta

SEQ ID NO: 400 - DNA, 20 bp, synthetic construct
  misc_feature 1..20: note = Antisense sequence to target splice site of proprocessed human dystrophin
  source 1..20: mol_type = other DNA
tgcctgtaagaacaaatatc

SEQ ID NO: 401 - DNA, 20 bp, synthetic construct
  misc_feature 1..20: note = Antisense sequence to target splice site of proprocessed human dystrophin
  source 1..20: mol_type = other DNA
attgttgcctgtaagaacaa

SEQ ID NO: 402 - DNA, 20 bp, synthetic construct
  misc_feature 1..20: note = Antisense sequence to target splice site of proprocessed human dystrophin
  source 1..20: mol_type = other DNA
cctgcattgttgcctgtaag

SEQ ID NO: 403 - DNA, 20 bp, synthetic construct
  misc_feature 1..20: note = Antisense sequence to target splice site of proprocessed human dystrophin
  source 1..20: mol_type = other DNA
caaatcctgcattgttgcct

SEQ ID NO: 404 - DNA, 20 bp, synthetic construct
  misc_feature 1..20: note = Antisense sequence to target splice site of proprocessed human dystrophin
  source 1..20: mol_type = other DNA
gcctctgttccaaatcctgc

SEQ ID NO: 405 - DNA, 20 bp, synthetic construct
  misc_feature 1..20: note = Antisense sequence to target splice site of proprocessed human dystrophin
  source 1..20: mol_type = other DNA
cttccaactggggacgcctc

SEQ ID NO: 406 - DNA, 20 bp, synthetic construct
  misc_feature 1..20: note = Antisense sequence to target splice site of proprocessed human dystrophin
  source 1..20: mol_type = other DNA
cagcggtaatgagttcttcc

SEQ ID NO: 407 - DNA, 20 bp, synthetic construct
  misc_feature 1..20: note = Antisense sequence to target splice site of proprocessed human dystrophin
  source 1..20: mol_type = other DNA
ttttcaaattttgggcagcg

SEQ ID NO: 408 - DNA, 20 bp, synthetic construct
  misc_feature 1..20: note = Antisense sequence to target splice site of proprocessed human dystrophin
  source 1..20: mol_type = other DNA
gattgctggtcttgtttttc

SEQ ID NO: 409 - DNA, 20 bp, synthetic construct
  misc_feature 1..20: note = Antisense sequence to target splice site of proprocessed human dystrophin
  source 1..20: mol_type = other DNA
ttgttctagcctcttgattg

SEQ ID NO: 410 - DNA, 20 bp, synthetic construct
  misc_feature 1..20: note = Antisense sequence to target splice site of proprocessed human dystrophin
  source 1..20: mol_type = other DNA
ttcgatccgtaatgattgtt

SEQ ID NO: 411 - DNA, 20 bp, synthetic construct
  misc_feature 1..20: note = Antisense sequence to target splice site of proprocessed human dystrophin
  source 1..20: mol_type = other DNA
cttacttcgatccgtaatga

SEQ ID NO: 412 - DNA, 20 bp, synthetic construct
  misc_feature 1..20: note = Antisense sequence to target splice site of proprocessed human dystrophin
  source 1..20: mol_type = other DNA
aaaaacttacttcgatccgt

SEQ ID NO: 413 - DNA, 20 bp, synthetic construct
  misc_feature 1..20: note = Antisense sequence to target splice site of proprocessed human dystrophin
  source 1..20: mol_type = other DNA
tgttaaaaaacttacttcga

SEQ ID NO: 414 - DNA, 20 bp, synthetic construct
  misc_feature 1..20: note = Antisense sequence to target splice site of proprocessed human dystrophin
  source 1..20: mol_type = other DNA
atgcttgttaaaaaacttac

SEQ ID NO: 415 - DNA, 20 bp, synthetic construct
  misc_feature 1..20: note = Antisense sequence to target splice site of proprocessed human dystrophin
  source 1..20: mol_type = other DNA
gtcccatgcttgttaaaaaa

SEQ ID NO: 416 - DNA, 25 bp, synthetic construct
  misc_feature 1..25: note = Antisense sequence to target splice site of proprocessed human dystrophin
  source 1..25: mol_type = other DNA
ctagaataaaaggaaaaataaatat

SEQ ID NO: 417 - DNA, 25 bp, synthetic construct
  misc_feature 1..25: note = Antisense sequence to target splice site of proprocessed human dystrophin
  source 1..25: mol_type = other DNA
aactagaataaaaggaaaaataaat

SEQ ID NO: 418 - DNA, 25 bp, synthetic construct
  misc_feature 1..25: note = Antisense sequence to target splice site of proprocessed human dystrophin
  source 1..25: mol_type = other DNA
ttcaactagaataaaaggaaaaata

SEQ ID NO: 419 - DNA, 25 bp, synthetic construct
  misc_feature 1..25: note = Antisense sequence to target splice site of proprocessed human dystrophin
  source 1..25: mol_type = other DNA
ctttcaactagaataaaaggaaaaa

SEQ ID NO: 420 - DNA, 25 bp, synthetic construct
  misc_feature 1..25: note = Antisense sequence to target splice site of proprocessed human dystrophin
  source 1..25: mol_type = other DNA
attctttcaactagaataaaaggaa

SEQ ID NO: 421 - DNA, 25 bp, synthetic construct
  misc_feature 1..25: note = Antisense sequence to target splice site of proprocessed human dystrophin
  source 1..25: mol_type = other DNA
gaattctttcaactagaataaaagg

SEQ ID NO: 422 - DNA, 25 bp, synthetic construct
  misc_feature 1..25: note = Antisense sequence to target splice site of proprocessed human dystrophin
  source 1..25: mol_type = other DNA
tctgaattctttcaactagaataaa

SEQ ID NO: 423 - DNA, 25 bp, synthetic construct
  misc_feature 1..25: note = Antisense sequence to target splice site of proprocessed human dystrophin
  source 1..25: mol_type = other DNA
attctgaattctttcaactagaata

SEQ ID NO: 424 - DNA, 25 bp, synthetic construct
  misc_feature 1..25: note = Antisense sequence to target splice site of proprocessed human dystrophin
  source 1..25: mol_type = other DNA
ctgattctgaattctttcaactaga

SEQ ID NO: 425 - DNA, 25 bp, synthetic construct
  misc_feature 1..25: note = Antisense sequence to target splice site of proprocessed human dystrophin
  source 1..25: mol_type = other DNA
cactgattctgaattctttcaacta

SEQ ID NO: 426 - DNA, 25 bp, synthetic construct
  misc_feature 1..25: note = Antisense sequence to target splice site of proprocessed human dystrophin
  source 1..25: mol_type = other DNA
tcccactgattctgaattctttcaa

SEQ ID NO: 427 - DNA, 25 bp, synthetic construct
  misc_feature 1..25: note = Antisense sequence to target splice site of proprocessed human dystrophin
  source 1..25: mol_type = other DNA
catcccactgattctgaattctttc

SEQ ID NO: 428 - DNA, 25 bp, synthetic construct
  misc_feature 1..25: note = Antisense sequence to target splice site of proprocessed human dystrophin
  source 1..25: mol_type = other DNA
tacttcatcccactgattctgaatt

SEQ ID NO: 429 - DNA, 25 bp, synthetic construct
  misc_feature 1..25: note = Antisense sequence to target splice site of proprocessed human dystrophin
  source 1..25: mol_type = other DNA
ctgaaggtgttcttgtacttcatcc

SEQ ID NO: 430 - DNA, 24 bp, synthetic construct
  misc_feature 1..24: note = Antisense sequence to target splice site of proprocessed human dystrophin
  source 1..24: mol_type = other DNA
cggttctgaaggtgttcttgtact

SEQ ID NO: 431 - DNA, 25 bp, synthetic construct
  misc_feature 1..25: note = Antisense sequence to target splice site of proprocessed human dystrophin
  source 1..25: mol_type = other DNA
ctgttgcctccggttctgaaggtgt

SEQ ID NO: 432 - DNA, 25 bp, synthetic construct
  misc_feature 1..25: note = Antisense sequence to target splice site of proprocessed human dystrophin
  source 1..25: mol_type = other DNA
tttcattcaactgttgcctccggtt

SEQ ID NO: 433 - DNA, 25 bp, synthetic construct
  misc_feature 1..25: note = Antisense sequence to target splice site of proprocessed human dystrophin
  source 1..25: mol_type = other DNA
taacatttcattcaactgttgcctc

SEQ ID NO: 434 - DNA, 25 bp, synthetic construct
  misc_feature 1..25: note = Antisense sequence to target splice site of proprocessed human dystrophin
  source 1..25: mol_type = other DNA
ttgtgttgaatcctttaacatttca

SEQ ID NO: 435 - DNA, 25 bp, synthetic construct
  misc_feature 1..25: note = Antisense sequence to target splice site of proprocessed human dystrophin
  source 1..25: mol_type = other DNA
tcttccttagcttccagccattgtg

SEQ ID NO: 436 - DNA, 25 bp, synthetic construct
  misc_feature 1..25: note = Antisense sequence to target splice site of proprocessed human dystrophin
  source 1..25: mol_type = other DNA
cttagcttccagccattgtgttgaa

SEQ ID NO: 437 - DNA, 25 bp, synthetic construct
  misc_feature 1..25: note = Antisense sequence to target splice site of proprocessed human dystrophin
  source 1..25: mol_type = other DNA
gtcctaagacctgctcagcttcttc

SEQ ID NO: 438 - DNA, 25 bp, synthetic construct
  misc_feature 1..25: note = Antisense sequence to target splice site of proprocessed human dystrophin
  source 1..25: mol_type = other DNA
ctgctcagcttcttccttagcttcc

SEQ ID NO: 439 - DNA, 25 bp, synthetic construct
  misc_feature 1..25: note = Antisense sequence to target splice site of proprocessed human dystrophin
  source 1..25: mol_type = other DNA
ctcaagcttggctctggcctgtcct

SEQ ID NO: 440 - DNA, 25 bp, synthetic construct
  misc_feature 1..25: note = Antisense sequence to target splice site of proprocessed human dystrophin
  source 1..25: mol_type = other DNA
ggcctgtcctaagacctgctcagct

SEQ ID NO: 441 - DNA, 25 bp, synthetic construct
  misc_feature 1..25: note = Antisense sequence to target splice site of proprocessed human dystrophin
  source 1..25: mol_type = other DNA
tagggaccctccttccatgactcaa

SEQ ID NO: 442 - DNA, 25 bp, synthetic construct
  misc_feature 1..25: note = Antisense sequence to target splice site of proprocessed human dystrophin
  source 1..25: mol_type = other DNA
tttggattgcatctactgtataggg

SEQ ID NO: 443 - DNA, 25 bp, synthetic construct
  misc_feature 1..25: note = Antisense sequence to target splice site of proprocessed human dystrophin
  source 1..25: mol_type = other DNA
accctccttccatgactcaagcttg

SEQ ID NO: 444 - DNA, 25 bp, synthetic construct
  misc_feature 1..25: note = Antisense sequence to target splice site of proprocessed human dystrophin
  source 1..25: mol_type = other DNA
cttggtttctgtgattttcttttgg

SEQ ID NO: 445 - DNA, 25 bp, synthetic construct
  misc_feature 1..25: note = Antisense sequence to target splice site of proprocessed human dystrophin
  source 1..25: mol_type = other DNA
atctactgtatagggaccctccttc

SEQ ID NO: 446 - DNA, 25 bp, synthetic construct
  misc_feature 1..25: note = Antisense sequence to target splice site of proprocessed human dystrophin
  source 1..25: mol_type = other DNA
ctaaccttggtttctgtgattttct

SEQ ID NO: 447 - DNA, 25 bp, synthetic construct
  misc_feature 1..25: note = Antisense sequence to target splice site of proprocessed human dystrophin
  source 1..25: mol_type = other DNA
tttcttttggattgcatctactgta

SEQ ID NO: 448 - DNA, 25 bp, synthetic construct
  misc_feature 1..25: note = Antisense sequence to target splice site of proprocessed human dystrophin
  source 1..25: mol_type = other DNA
tgatactaaccttggtttctgtgat

SEQ ID NO: 449 - DNA, 25 bp, synthetic construct
  misc_feature 1..25: note = Antisense sequence to target splice site of proprocessed human dystrophin
  source 1..25: mol_type = other DNA
atctttgatactaaccttggtttct

SEQ ID NO: 450 - DNA, 25 bp, synthetic construct
  misc_feature 1..25: note = Antisense sequence to target splice site of proprocessed human dystrophin
  source 1..25: mol_type = other DNA
aaggtatctttgatactaaccttgg

SEQ ID NO: 451 - DNA, 25 bp, synthetic construct
  misc_feature 1..25: note = Antisense sequence to target splice site of proprocessed human dystrophin
  source 1..25: mol_type = other DNA
ttaaaaaggtatctttgatactaac

SEQ ID NO: 452 - DNA, 20 bp, synthetic construct
  misc_feature 1..20: note = Antisense sequence to target splice site of proprocessed human dystrophin
  source 1..20: mol_type = other DNA
ataaaaggaaaaataaatat

SEQ ID NO: 453 - DNA, 20 bp, synthetic construct
  misc_feature 1..20: note = Antisense sequence to target splice site of proprocessed human dystrophin
  source 1..20: mol_type = other DNA
gaataaaaggaaaaataaat

SEQ ID NO: 454 - DNA, 20 bp, synthetic construct
  misc_feature 1..20: note = Antisense sequence to target splice site of proprocessed human dystrophin
  source 1..20: mol_type = other DNA
aactagaataaaaggaaaaa

SEQ ID NO: 455 - DNA, 20 bp, synthetic construct
  misc_feature 1..20: note = Antisense sequence to target splice site of proprocessed human dystrophin
  source 1..20: mol_type = other DNA
ctttcaactagaataaaagg

SEQ ID NO: 456 - DNA, 20 bp, synthetic construct
  misc_feature 1..20: note = Antisense sequence to target splice site of proprocessed human dystrophin
  source 1..20: mol_type = other DNA
gaattctttcaactagaata

SEQ ID NO: 457 - DNA, 20 bp, synthetic construct
  misc_feature 1..20: note = Antisense sequence to target splice site of proprocessed human dystrophin
  source 1..20: mol_type = other DNA
attctgaattctttcaacta

SEQ ID NO: 458 - DNA, 20 bp, synthetic construct
  misc_feature 1..20: note = Antisense sequence to target splice site of proprocessed human dystrophin
  source 1..20: mol_type = other DNA
tacttcatcccactgattct

SEQ ID NO: 459 - DNA, 19 bp, synthetic construct
  misc_feature 1..19: note = Antisense sequence to target splice site of proprocessed human dystrophin
  source 1..19: mol_type = other DNA
ctgaaggtgttcttgtact

SEQ ID NO: 460 - DNA, 20 bp, synthetic construct
  misc_feature 1..20: note = Antisense sequence to target splice site of proprocessed human dystrophin
  source 1..20: mol_type = other DNA
ctgttgcctccggttctgaa

SEQ ID NO: 461 - DNA, 20 bp, synthetic construct
  misc_feature 1..20: note = Antisense sequence to target splice site of proprocessed human dystrophin
  source 1..20: mol_type = other DNA
taacatttcattcaactgtt

SEQ ID NO: 462 - DNA, 20 bp, synthetic construct
  misc_feature 1..20: note = Antisense sequence to target splice site of proprocessed human dystrophin
  source 1..20: mol_type = other DNA
ttgtgttgaatcctttaaca

SEQ ID NO: 463 - DNA, 20 bp, synthetic construct
  misc_feature 1..20: note = Antisense sequence to target splice site of proprocessed human dystrophin
  source 1..20: mol_type = other DNA
cttagcttccagccattgtg

SEQ ID NO: 464 - DNA, 20 bp, synthetic construct
  misc_feature 1..20: note = Antisense sequence to target splice site of proprocessed human dystrophin
  source 1..20: mol_type = other DNA
ctgctcagcttcttccttag

SEQ ID NO: 465 - DNA, 20 bp, synthetic construct
  misc_feature 1..20: note = Antisense sequence to target splice site of proprocessed human dystrophin
  source 1..20: mol_type = other DNA
ggcctgtcctaagacctgct

SEQ ID NO: 466 - DNA, 20 bp, synthetic construct
  misc_feature 1..20: note = Antisense sequence to target splice site of proprocessed human dystrophin
  source 1..20: mol_type = other DNA
ctcaagcttggctctggcct

SEQ ID NO: 467 - DNA, 20 bp, synthetic construct
  misc_feature 1..20: note = Antisense sequence to target splice site of proprocessed human dystrophin
  source 1..20: mol_type = other DNA
accctccttccatgactcaa

SEQ ID NO: 468 - DNA, 20 bp, synthetic construct
  misc_feature 1..20: note = Antisense sequence to target splice site of proprocessed human dystrophin
  source 1..20: mol_type = other DNA
atctactgtatagggaccct

SEQ ID NO: 469 - DNA, 20 bp, synthetic construct
  misc_feature 1..20: note = Antisense sequence to target splice site of proprocessed human dystrophin
  source 1..20: mol_type = other DNA
tttcttttggattgcatcta

SEQ ID NO: 470 - DNA, 20 bp, synthetic construct
  misc_feature 1..20: note = Antisense sequence to target splice site of proprocessed human dystrophin
  source 1..20: mol_type = other DNA
cttggtttctgtgattttct

SEQ ID NO: 471 - DNA, 20 bp, synthetic construct
  misc_feature 1..20: note = Antisense sequence to target splice site of proprocessed human dystrophin
  source 1..20: mol_type = other DNA
ctaaccttggtttctgtgat

SEQ ID NO: 472 - DNA, 20 bp, synthetic construct
  misc_feature 1..20: note = Antisense sequence to target splice site of proprocessed human dystrophin
  source 1..20: mol_type = other DNA
tgatactaaccttggtttct

SEQ ID NO: 473 - DNA, 20 bp, synthetic construct
  misc_feature 1..20: note = Antisense sequence to target splice site of proprocessed human dystrophin
  source 1..20: mol_type = other DNA
atctttgatactaaccttgg

SEQ ID NO: 474 - DNA, 20 bp, synthetic construct
  misc_feature 1..20: note = Antisense sequence to target splice site of proprocessed human dystrophin
  source 1..20: mol_type = other DNA
aaggtatctttgatactaac

SEQ ID NO: 475 - DNA, 20 bp, synthetic construct
  misc_feature 1..20: note = Antisense sequence to target splice site of proprocessed human dystrophin
  source 1..20: mol_type = other DNA
ttaaaaaggtatctttgata

SEQ ID NO: 476 - DNA, 24 bp, synthetic construct
  misc_feature 1..24: note = Antisense sequence to target splice site of proprocessed human dystrophin
  source 1..24: mol_type = other DNA
ctatagatttttatgagaaagaga

SEQ ID NO: 477 - DNA, 25 bp, synthetic construct
  misc_feature 1..25: note = Antisense sequence to target splice site of proprocessed human dystrophin
  source 1..25: mol_type = other DNA
aactgctatagatttttatgagaaa

SEQ ID NO: 478 - DNA, 25 bp, synthetic construct
  misc_feature 1..25: note = Antisense sequence to target splice site of proprocessed human dystrophin
  source 1..25: mol_type = other DNA
tggccaactgctatagatttttatg

SEQ ID NO: 479 - DNA, 25 bp, synthetic construct
  misc_feature 1..25: note = Antisense sequence to target splice site of proprocessed human dystrophin
  source 1..25: mol_type = other DNA
gtctttggccaactgctatagattt

SEQ ID NO: 480 - DNA, 25 bp, synthetic construct
  misc_feature 1..25: note = Antisense sequence to target splice site of proprocessed human dystrophin
  source 1..25: mol_type = other DNA
cggaggtctttggccaactgctata

SEQ ID NO: 481 - DNA, 25 bp, synthetic construct
  misc_feature 1..25: note = Antisense sequence to target splice site of proprocessed human dystrophin
  source 1..25: mol_type = other DNA
actggcggaggtctttggccaactg

SEQ ID NO: 482 - DNA, 25 bp, synthetic construct
  misc_feature 1..25: note = Antisense sequence to target splice site of proprocessed human dystrophin
  source 1..25: mol_type = other DNA
tttgtctgccactggcggaggtctt

SEQ ID NO: 483 - DNA, 25 bp, synthetic construct
  misc_feature 1..25: note = Antisense sequence to target splice site of proprocessed human dystrophin
  source 1..25: mol_type = other DNA
agtcatttgccacatctacatttgt

SEQ ID NO: 484 - DNA, 25 bp, synthetic construct
  misc_feature 1..25: note = Antisense sequence to target splice site of proprocessed human dystrophin
  source 1..25: mol_type = other DNA
tttgccacatctacatttgtctgcc

SEQ ID NO: 485 - DNA, 25 bp, synthetic construct
  misc_feature 1..25: note = Antisense sequence to target splice site of proprocessed human dystrophin
  source 1..25: mol_type = other DNA
ccggagaagtttcagggccaagtca

SEQ ID NO: 486 - DNA, 25 bp, synthetic construct
  misc_feature 1..25: note = Antisense sequence to target splice site of proprocessed human dystrophin
  source 1..25: mol_type = other DNA
gtatcatctgcagaataatcccgga

SEQ ID NO: 487 - DNA, 25 bp, synthetic construct
  misc_feature 1..25: note = Antisense sequence to target splice site of proprocessed human dystrophin
  source 1..25: mol_type = other DNA
taatcccggagaagtttcagggcca

SEQ ID NO: 488 - DNA, 25 bp, synthetic construct
  misc_feature 1..25: note = Antisense sequence to target splice site of proprocessed human dystrophin
  source 1..25: mol_type = other DNA
ttatcatgtggacttttctggtatc

SEQ ID NO: 489 - DNA, 25 bp, synthetic construct
  misc_feature 1..25: note = Antisense sequence to target splice site of proprocessed human dystrophin
  source 1..25: mol_type = other DNA
agaggcattgatattctctgttatc

SEQ ID NO: 490 - DNA, 25 bp, synthetic construct
  misc_feature 1..25: note = Antisense sequence to target splice site of proprocessed human dystrophin
  source 1..25: mol_type = other DNA
atgtggacttttctggtatcatctg

SEQ ID NO: 491 - DNA, 25 bp, synthetic construct
  misc_feature 1..25: note = Antisense sequence to target splice site of proprocessed human dystrophin
  source 1..25: mol_type = other DNA
cttttatgaatgcttctccaagagg

SEQ ID NO: 492 - DNA, 25 bp, synthetic construct
  misc_feature 1..25: note = Antisense sequence to target splice site of proprocessed human dystrophin
  source 1..25: mol_type = other DNA
atattctctgttatcatgtggactt

SEQ ID NO: 493 - DNA, 25 bp, synthetic construct
  misc_feature 1..25: note = Antisense sequence to target splice site of proprocessed human dystrophin
  source 1..25: mol_type = other DNA
cataccttttatgaatgcttctcca

SEQ ID NO: 494 - DNA, 25 bp, synthetic construct
  misc_feature 1..25: note = Antisense sequence to target splice site of proprocessed human dystrophin
  source 1..25: mol_type = other DNA
ctccaagaggcattgatattctctg

SEQ ID NO: 495 - DNA, 25 bp, synthetic construct
  misc_feature 1..25: note = Antisense sequence to target splice site of proprocessed human dystrophin
  source 1..25: mol_type = other DNA
taattcataccttttatgaatgctt

SEQ ID NO: 496 - DNA, 25 bp, synthetic construct
  misc_feature 1..25: note = Antisense sequence to target splice site of proprocessed human dystrophin
  source 1..25: mol_type = other DNA
cttttatgaatgcttctccaagagg

SEQ ID NO: 497 - DNA, 25 bp, synthetic construct
  misc_feature 1..25: note = Antisense sequence to target splice site of proprocessed human dystrophin
  source 1..25: mol_type = other DNA
taatgtaattcataccttttatgaa

SEQ ID NO: 498 - DNA, 25 bp, synthetic construct
  misc_feature 1..25: note = Antisense sequence to target splice site of proprocessed human dystrophin
  source 1..25: mol_type = other DNA
agaaataatgtaattcatacctttt

SEQ ID NO: 499 - DNA, 25 bp, synthetic construct
  misc_feature 1..25: note = Antisense sequence to target splice site of proprocessed human dystrophin
  source 1..25: mol_type = other DNA
gttttagaaataatgtaattcatac

SEQ ID NO: 500 - DNA, 19 bp, synthetic construct
  misc_feature 1..19: note = Antisense sequence to target splice site of proprocessed human dystrophin
  source 1..19: mol_type = other DNA
gatttttatgagaaagaga

SEQ ID NO: 501 - DNA, 20 bp, synthetic construct
  misc_feature 1..20: note = Antisense sequence to target splice site of proprocessed human dystrophin
  source 1..20: mol_type = other DNA
ctatagatttttatgagaaa

SEQ ID NO: 502 - DNA, 20 bp, synthetic construct
  misc_feature 1..20: note = Antisense sequence to target splice site of proprocessed human dystrophin
  source 1..20: mol_type = other DNA
aactgctatagatttttatg

SEQ ID NO: 503 - DNA, 20 bp, synthetic construct
  misc_feature 1..20: note = Antisense sequence to target splice site of proprocessed human dystrophin
  source 1..20: mol_type = other DNA
tggccaactgctatagattt

SEQ ID NO: 504 - DNA, 20 bp, synthetic construct
  misc_feature 1..20: note = Antisense sequence to target splice site of proprocessed human dystrophin
  source 1..20: mol_type = other DNA
gtctttggccaactgctata

SEQ ID NO: 505 - DNA, 20 bp, synthetic construct
  misc_feature 1..20: note = Antisense sequence to target splice site of proprocessed human dystrophin
  source 1..20: mol_type = other DNA
cggaggtctttggccaactg

SEQ ID NO: 506 - DNA, 20 bp, synthetic construct
  misc_feature 1..20: note = Antisense sequence to target splice site of proprocessed human dystrophin
  source 1..20: mol_type = other DNA
tttgtctgccactggcggag

SEQ ID NO: 507 - DNA, 20 bp, synthetic construct
  misc_feature 1..20: note = Antisense sequence to target splice site of proprocessed human dystrophin
  source 1..20: mol_type = other DNA
tttgccacatctacatttgt

SEQ ID NO: 508 - DNA, 20 bp, synthetic construct
  misc_feature 1..20: note = Antisense sequence to target splice site of proprocessed human dystrophin
  source 1..20: mol_type = other DNA
ttcagggccaagtcatttgc

SEQ ID NO: 509 - DNA, 20 bp, synthetic construct
  misc_feature 1..20: note = Antisense sequence to target splice site of proprocessed human dystrophin
  source 1..20: mol_type = other DNA
taatcccggagaagtttcag

SEQ ID NO: 510 - DNA, 20 bp, synthetic construct
  misc_feature 1..20: note = Antisense sequence to target splice site of proprocessed human dystrophin
  source 1..20: mol_type = other DNA
gtatcatctgcagaataatc

SEQ ID NO: 511 - DNA, 20 bp, synthetic construct
  misc_feature 1..20: note = Antisense sequence to target splice site of proprocessed human dystrophin
  source 1..20: mol_type = other DNA
atgtggacttttctggtatc

SEQ ID NO: 512 - DNA, 20 bp, synthetic construct
  misc_feature 1..20: note = Antisense sequence to target splice site of proprocessed human dystrophin
  source 1..20: mol_type = other DNA
atattctctgttatcatgtg

SEQ ID NO: 513 - DNA, 20 bp, synthetic construct
  misc_feature 1..20: note = Antisense sequence to target splice site of proprocessed human dystrophin
  source 1..20: mol_type = other DNA
ctccaagaggcattgatatt

SEQ ID NO: 514 - DNA, 20 bp, synthetic construct
  misc_feature 1..20: note = Antisense sequence to target splice site of proprocessed human dystrophin
  source 1..20: mol_type = other DNA
cttttatgaatgcttctcca

SEQ ID NO: 515 - DNA, 20 bp, synthetic construct
  misc_feature 1..20: note = Antisense sequence to target splice site of proprocessed human dystrophin
  source 1..20: mol_type = other DNA
cataccttttatgaatgctt

SEQ ID NO: 516 - DNA, 20 bp, synthetic construct
  misc_feature 1..20: note = Antisense sequence to target splice site of proprocessed human dystrophin
  source 1..20: mol_type = other DNA
taattcataccttttatgaa

SEQ ID NO: 517 - DNA, 20 bp, synthetic construct
  misc_feature 1..20: note = Antisense sequence to target splice site of proprocessed human dystrophin
  source 1..20: mol_type = other DNA
taatgtaattcatacctttt

SEQ ID NO: 518 - DNA, 20 bp, synthetic construct
  misc_feature 1..20: note = Antisense sequence to target splice site of proprocessed human dystrophin
  source 1..20: mol_type = other DNA
agaaataatgtaattcatac

SEQ ID NO: 519 - DNA, 20 bp, synthetic construct
  misc_feature 1..20: note = Antisense sequence to target splice site of proprocessed human dystrophin
  source 1..20: mol_type = other DNA
gttttagaaataatgtaatt

SEQ ID NO: 520 - DNA, 25 bp, synthetic construct
  misc_feature 1..25: note = Antisense sequence to target splice site of proprocessed human dystrophin
  source 1..25: mol_type = other DNA
ctgcaaaggaccaaatgttcagatg

SEQ ID NO: 521 - DNA, 25 bp, synthetic construct
  misc_feature 1..25: note = Antisense sequence to target splice site of proprocessed human dystrophin
  source 1..25: mol_type = other DNA
tcaccctgcaaaggaccaaatgttc

SEQ ID NO: 522 - DNA, 25 bp, synthetic construct
  misc_feature 1..25: note = Antisense sequence to target splice site of proprocessed human dystrophin
  source 1..25: mol_type = other DNA
ctcactcaccctgcaaaggaccaaa

SEQ ID NO: 523 - DNA, 25 bp, synthetic construct
  misc_feature 1..25: note = Antisense sequence to target splice site of proprocessed human dystrophin
  source 1..25: mol_type = other DNA
tctcgctcactcaccctgcaaagga

SEQ ID NO: 524 - DNA, 25 bp, synthetic construct
  misc_feature 1..25: note = Antisense sequence to target splice site of proprocessed human dystrophin
  source 1..25: mol_type = other DNA
cagcctctcgctcactcaccctgca

SEQ ID NO: 525 - DNA, 25 bp, synthetic construct
  misc_feature 1..25: note = Antisense sequence to target splice site of proprocessed human dystrophin
  source 1..25: mol_type = other DNA
caaagcagcctctcgctcactcacc

SEQ ID NO: 526 - DNA, 25 bp, synthetic construct
  misc_feature 1..25: note = Antisense sequence to target splice site of proprocessed human dystrophin
  source 1..25: mol_type = other DNA
tcttccaaagcagcctctcgctcac

SEQ ID NO: 527 - DNA, 25 bp, synthetic construct
  misc_feature 1..25: note = Antisense sequence to target splice site of proprocessed human dystrophin
  source 1..25: mol_type = other DNA
tctatgagtttcttccaaagcagcc

SEQ ID NO: 528 - DNA, 25 bp, synthetic construct
  misc_feature 1..25: note = Antisense sequence to target splice site of proprocessed human dystrophin
  source 1..25: mol_type = other DNA
gttgcagtaatctatgagtttcttc

SEQ ID NO: 529 - DNA, 25 bp, synthetic construct
  misc_feature 1..25: note = Antisense sequence to target splice site of proprocessed human dystrophin
  source 1..25: mol_type = other DNA
gaactgttgcagtaatctatgagtt

SEQ ID NO: 530 - DNA, 25 bp, synthetic construct
  misc_feature 1..25: note = Antisense sequence to target splice site of proprocessed human dystrophin
  source 1..25: mol_type = other DNA
ttccaggtccagggggaactgttgc

SEQ ID NO: 531 - DNA, 25 bp, synthetic construct
  misc_feature 1..25: note = Antisense sequence to target splice site of proprocessed human dystrophin
  source 1..25: mol_type = other DNA
gtaagccaggcaagaaacttttcca

SEQ ID NO: 532 - DNA, 25 bp, synthetic construct
  misc_feature 1..25: note = Antisense sequence to target splice site of proprocessed human dystrophin
  source 1..25: mol_type = other DNA
ccaggcaagaaacttttccaggtcc

SEQ ID NO: 533 - DNA, 25 bp, synthetic construct
  misc_feature 1..25: note = Antisense sequence to target splice site of proprocessed human dystrophin
  source 1..25: mol_type = other DNA
tggcagttgtttcagcttctgtaag

SEQ ID NO: 534 - DNA, 25 bp, synthetic construct
  misc_feature 1..25: note = Antisense sequence to target splice site of proprocessed human dystrophin
  source 1..25: mol_type = other DNA
ggtagcatcctgtaggacattggca

SEQ ID NO: 535 - DNA, 25 bp, synthetic construct
  misc_feature 1..25: note = Antisense sequence to target splice site of proprocessed human dystrophin
  source 1..25: mol_type = other DNA
gacattggcagttgtttcagcttct

SEQ ID NO: 536 - DNA, 25 bp, synthetic construct
  misc_feature 1..25: note = Antisense sequence to target splice site of proprocessed human dystrophin
  source 1..25: mol_type = other DNA
tctaggagcctttccttacgggtag

SEQ ID NO: 537 - DNA, 25 bp, synthetic construct
  misc_feature 1..25: note = Antisense sequence to target splice site of proprocessed human dystrophin
  source 1..25: mol_type = other DNA
cttttactcccttggagtcttctag

SEQ ID NO: 538 - DNA, 25 bp, synthetic construct
  misc_feature 1..25: note = Antisense sequence to target splice site of proprocessed human dystrophin
  source 1..25: mol_type = other DNA
gagcctttccttacgggtagcatcc

SEQ ID NO: 539 - DNA, 25 bp, synthetic construct
  misc_feature 1..25: note = Antisense sequence to target splice site of proprocessed human dystrophin
  source 1..25: mol_type = other DNA
ttgccattgtttcatcagctctttt

SEQ ID NO: 540 - DNA, 25 bp, synthetic construct
  misc_feature 1..25: note = Antisense sequence to target splice site of proprocessed human dystrophin
  source 1..25: mol_type = other DNA
cttggagtcttctaggagcctttcc

SEQ ID NO: 541 - DNA, 25 bp, synthetic construct
  misc_feature 1..25: note = Antisense sequence to target splice site of proprocessed human dystrophin
  source 1..25: mol_type = other DNA
cttacttgccattgtttcatcagct

SEQ ID NO: 542 - DNA, 25 bp, synthetic construct
  misc_feature 1..25: note = Antisense sequence to target splice site of proprocessed human dystrophin
  source 1..25: mol_type = other DNA
cagctcttttactcccttggagtct

SEQ ID NO: 543 - DNA, 25 bp, synthetic construct
  misc_feature 1..25: note = Antisense sequence to target splice site of proprocessed human dystrophin
  source 1..25: mol_type = other DNA
cctgacttacttgccattgtttcat

SEQ ID NO: 544 - DNA, 25 bp, synthetic construct
  misc_feature 1..25: note = Antisense sequence to target splice site of proprocessed human dystrophin
  source 1..25: mol_type = other DNA
aaatgcctgacttacttgccattgt

SEQ ID NO: 545 - DNA, 25 bp, synthetic construct
  misc_feature 1..25: note = Antisense sequence to target splice site of proprocessed human dystrophin
  source 1..25: mol_type = other DNA
agcggaaatgcctgacttacttgcc

SEQ ID NO: 546 - DNA, 25 bp, synthetic construct
  misc_feature 1..25: note = Antisense sequence to target splice site of proprocessed human dystrophin
  source 1..25: mol_type = other DNA
gctaaagcggaaatgcctgacttac

SEQ ID NO: 547 - DNA, 20 bp, synthetic construct
  misc_feature 1..20: note = Antisense sequence to target splice site of proprocessed human dystrophin
  source 1..20: mol_type = other DNA
aaggaccaaatgttcagatg

SEQ ID NO: 548 - DNA, 20 bp, synthetic construct
  misc_feature 1..20: note = Antisense sequence to target splice site of proprocessed human dystrophin
  source 1..20: mol_type = other DNA
ctgcaaaggaccaaatgttc

SEQ ID NO: 549 - DNA, 20 bp, synthetic construct
  misc_feature 1..20: note = Antisense sequence to target splice site of proprocessed human dystrophin
  source 1..20: mol_type = other DNA
tcaccctgcaaaggaccaaa

SEQ ID NO: 550 - DNA, 20 bp, synthetic construct
  misc_feature 1..20: note = Antisense sequence to target splice site of proprocessed human dystrophin
  source 1..20: mol_type = other DNA
ctcactcaccctgcaaagga

SEQ ID NO: 551 - DNA, 20 bp, synthetic construct
  misc_feature 1..20: note = Antisense sequence to target splice site of proprocessed human dystrophin
  source 1..20: mol_type = other DNA
tctcgctcactcaccctgca

SEQ ID NO: 552 - DNA, 20 bp, synthetic construct
  misc_feature 1..20: note = Antisense sequence to target splice site of proprocessed human dystrophin
  source 1..20: mol_type = other DNA
cagcctctcgctcactcacc

SEQ ID NO: 553 - DNA, 20 bp, synthetic construct
  misc_feature 1..20: note = Antisense sequence to target splice site of proprocessed human dystrophin
  source 1..20: mol_type = other DNA
caaagcagcctctcgctcac

SEQ ID NO: 554 - DNA, 20 bp, synthetic construct
  misc_feature 1..20: note = Antisense sequence to target splice site of proprocessed human dystrophin
  source 1..20: mol_type = other DNA
tctatgagtttcttccaaag

SEQ ID NO: 555 - DNA, 20 bp, synthetic construct
  misc_feature 1..20: note = Antisense sequence to target splice site of proprocessed human dystrophin
  source 1..20: mol_type = other DNA
gaactgttgcagtaatctat

SEQ ID NO: 556 - DNA, 20 bp, synthetic construct
  misc_feature 1..20: note = Antisense sequence to target splice site of proprocessed human dystrophin
  source 1..20: mol_type = other DNA
ttccaggtccagggggaact

SEQ ID NO: 557 - DNA, 20 bp, synthetic construct
  misc_feature 1..20: note = Antisense sequence to target splice site of proprocessed human dystrophin
  source 1..20: mol_type = other DNA
ccaggcaagaaacttttcca

SEQ ID NO: 558 - DNA, 20 bp, synthetic construct
  misc_feature 1..20: note = Antisense sequence to target splice site of proprocessed human dystrophin
  source 1..20: mol_type = other DNA
ttcagcttctgtaagccagg

SEQ ID NO: 559 - DNA, 20 bp, synthetic construct
  misc_feature 1..20: note = Antisense sequence to target splice site of proprocessed human dystrophin
  source 1..20: mol_type = other DNA
gacattggcagttgtttcag

SEQ ID NO: 560 - DNA, 20 bp, synthetic construct
  misc_feature 1..20: note = Antisense sequence to target splice site of proprocessed human dystrophin
  source 1..20: mol_type = other DNA
ggtagcatcctgtaggacat

SEQ ID NO: 561 - DNA, 20 bp, synthetic construct
  misc_feature 1..20: note = Antisense sequence to target splice site of proprocessed human dystrophin
  source 1..20: mol_type = other DNA
gagcctttccttacgggtag

SEQ ID NO: 562 - DNA, 20 bp, synthetic construct
  misc_feature 1..20: note = Antisense sequence to target splice site of proprocessed human dystrophin
  source 1..20: mol_type = other DNA
cttggagtcttctaggagcc

SEQ ID NO: 563 - DNA, 20 bp, synthetic construct
  misc_feature 1..20: note = Antisense sequence to target splice site of proprocessed human dystrophin
  source 1..20: mol_type = other DNA
cagctcttttactcccttgg

SEQ ID NO: 564 - DNA, 20 bp, synthetic construct
  misc_feature 1..20: note = Antisense sequence to target splice site of proprocessed human dystrophin
  source 1..20: mol_type = other DNA
ttgccattgtttcatcagct

SEQ ID NO: 565 - DNA, 20 bp, synthetic construct
  misc_feature 1..20: note = Antisense sequence to target splice site of proprocessed human dystrophin
  source 1..20: mol_type = other DNA
cttacttgccattgtttcat

SEQ ID NO: 566 - DNA, 20 bp, synthetic construct
  misc_feature 1..20: note = Antisense sequence to target splice site of proprocessed human dystrophin
  source 1..20: mol_type = other DNA
cctgacttacttgccattgt

SEQ ID NO: 567 - DNA, 20 bp, synthetic construct
  misc_feature 1..20: note = Antisense sequence to target splice site of proprocessed human dystrophin
  source 1..20: mol_type = other DNA
aaatgcctgacttacttgcc

SEQ ID NO: 568 - DNA, 20 bp, synthetic construct
  misc_feature 1..20: note = Antisense sequence to target splice site of proprocessed human dystrophin
  source 1..20: mol_type = other DNA
agcggaaatgcctgacttac

SEQ ID NO: 569 - DNA, 20 bp, synthetic construct
  misc_feature 1..20: note = Antisense sequence to target splice site of proprocessed human dystrophin
  source 1..20: mol_type = other DNA
gctaaagcggaaatgcctga

SEQ ID NO: 570 - protein, 10 aa, synthetic construct
  REGION 1..10: note = Peptide Transporter for Intracellular Delivery of PMO
RRRQRRKKRC

SEQ ID NO: 571 - protein, 12 aa, synthetic construct
  REGION 1..12: note = Peptide Transporter for Intracellular Delivery of PMO
RRRRRRRRRFFC

SEQ ID NO: 572 - protein, 13 aa, synthetic construct
  REGION 1..13: note = Peptide Transporter for Intracellular Delivery of PMO
  MOD_RES 3: note = X = 6-aminohexanoic acid
  MOD_RES 13: note = X = beta alanine
  MOD_RES 6: note = X = 6-aminohexanoic acid
  MOD_RES 9: note = X = 6-aminohexanoic acid
  MOD_RES 12: note = X = 6-aminohexanoic acid
RRXRRXRRXRRXX

SEQ ID NO: 573 - protein, 14 aa, synthetic construct
  REGION 1..14: note = Peptide Transporter for Intracellular Delivery of PMO
  MOD_RES 2: note = X = 6-aminohexanoic acid
  MOD_RES 14: note = X = beta alanine
  MOD_RES 5: note = X = 6-aminohexanoic acid
  MOD_RES 8: note = X = 6-aminohexanoic acid
  MOD_RES 11: note = X = 6-aminohexanoic acid
  MOD_RES 13: note = X = 6-aminohexanoic acid
RXRRXRRXRRXRXX

SEQ ID NO: 574 - protein, 14 aa, synthetic construct
  REGION 1..14: note = Peptide Transporter for Intracellular Delivery of PMO
  MOD_RES 1: note = X = 6-aminohexanoic acid
  MOD_RES 14: note = X = beta alanine
  MOD_RES 4: note = X = 6-aminohexanoic acid
  MOD_RES 10: note = X = 6-aminohexanoic acid
  MOD_RES 7: note = X = 6-aminohexanoic acid
  MOD_RES 13: note = X = 6-aminohexanoic acid
XRRXRRXRRXRRXX

SEQ ID NO: 575 - protein, 13 aa, synthetic construct
  REGION 1..13: note = Peptide Transporter for Intracellular Delivery of PMO
  MOD_RES 2: note = X = 6-aminohexanoic acid
  MOD_RES 13: note = X = beta alanine
  MOD_RES 4: note = X = 6-aminohexanoic acid
  MOD_RES 6: note = X = 6-aminohexanoic acid
  MOD_RES 8: note = X = 6-aminohexanoic acid
  MOD_RES 10: note = X = 6-aminohexanoic acid
  MOD_RES 12: note = X = 6-aminohexanoic acid
RXRXRXRXRXRXX

SEQ ID NO: 576 - protein, 17 aa, synthetic construct
  REGION 1..17: note = Peptide Transporter for Intracellular Delivery of PMO
  MOD_RES 2: note = X = 6-aminohexanoic acid
  MOD_RES 17: note = X = beta alanine
  MOD_RES 4: note = X = 6-aminohexanoic acid
  MOD_RES 6: note = X = 6-aminohexanoic acid
  MOD_RES 8: note = X = 6-aminohexanoic acid
  MOD_RES 10: note = X = 6-aminohexanoic acid
  MOD_RES 12: note = X = 6-aminohexanoic acid
  MOD_RES 14: note = X = 6-aminohexanoic acid
  MOD_RES 16: note = X = 6-aminohexanoic acid
RXRXRXRXRXRXRXRXX

SEQ ID NO: 577 - protein, 17 aa, synthetic construct
  REGION 1..17: note = Peptide Transporter for Intracellular Delivery of PMO
  MOD_RES 2: note = X = 6-aminohexanoic acid
  MOD_RES 17: note = X = beta alanine
  MOD_RES 5: note = X = 6-aminohexanoic acid
  MOD_RES 8: note = X = 6-aminohexanoic acid
  MOD_RES 11: note = X = 6-aminohexanoic acid
  MOD_RES 14: note = X = 6-aminohexanoic acid
  MOD_RES 16: note = X = 6-aminohexanoic acid
RXRRXRRXRRXRRXRXX

SEQ ID NO: 578 - protein, 14 aa, synthetic construct
  REGION 1..14: note = Peptide Transporter for Intracellular Delivery of PMO
  MOD_RES 2: note = X = 6-aminohexanoic acid
  MOD_RES 5: note = X = beta alanine
  MOD_RES 11: note = X = beta alanine
  MOD_RES 8: note = X = 6-aminohexanoic acid
  MOD_RES 14: note = X = beta alanine
  MOD_RES 13: note = X = 6-aminohexanoic acid
RXRRXRRXRRXRXX

SEQ ID NO: 579 - protein, 7 aa, synthetic construct
  REGION 1..7: note = Peptide Transporter for Intracellular Delivery of PMO
ASSLNIA

SEQ ID NO: 580 - protein, 9 aa, synthetic construct
  REGION 1..9: note = Cell Penetrating Peptide / Homing Peptide / PMO Conjugates
  VARIANT 8: note = X = a neutral amino acid C(=O) (CHR)n NH-, where each R is independently H or methyl
  MOD_RES 9: note = X = Beta alanine
  REPEAT 8: note = wherein (CHR) can be repeated between 2 to 7 times
ASSLNIAXX

SEQ ID NO: 581 - protein, 22 aa, synthetic construct
  REGION 1..22: note = Cell Penetrating Peptide / Homing Peptide / PMO Conjugates
  VARIANT 2: note = X = a neutral amino acid C(=O) (CHR)n NH-, where each R is independently H or methyl
  MOD_RES 5: note = X = beta alanine
  MOD_RES 11: note = X = beta alanine
  VARIANT 8: note = X = a neutral amino acid C(=O) (CHR)n NH-, where each R is independently H or methyl
  MOD_RES 14: note = X = beta alanine
  VARIANT 13: note = X = a neutral amino acid C(=O) (CHR)n NH-, where each R is independently H or methyl
  VARIANT 22: note = X = a neutral amino acid C(=O) (CHR)n NH-, where each R is independently H or methyl
  REPEAT 8: note = wherein (CHR) can be repeated between 2 to 7 times
  REPEAT 2: note = wherein (CHR) can be repeated between 2 to 7 times
  REPEAT 13: note = wherein (CHR) can be repeated between 2 to 7 times
  REPEAT 22: note = wherein (CHR) can be repeated between 2 to 7 times
RXRRXRRXRRXRXXASSLNIAX

SEQ ID NO: 582 - protein, 21 aa, synthetic construct
  REGION 1..21: note = Cell Penetrating Peptide / Homing Peptide / PMO Conjugates
  VARIANT 8: note = X = a neutral amino acid C(=O) (CHR)n NH-, where each R is independently H or methyl
  MOD_RES 13: note = X = beta alanine
  MOD_RES 19: note = X = beta alanine
  VARIANT 16: note = X = a neutral amino acid C(=O) (CHR)n NH-, where each R is independently H or methyl
  MOD_RES 21: note = X = beta alanine
  VARIANT 10: note = X = a neutral amino acid C(=O) (CHR)n NH-, where each R is independently H or methyl
  REPEAT 8: note = wherein (CHR) can be repeated between 2 to 7 times
  REPEAT 10: note = wherein (CHR) can be repeated between 2 to 7 times
  REPEAT 16: note = wherein (CHR) can be repeated between 2 to 7 times
ASSLNIAXRXRRXRRXRRXRX

SEQ ID NO: 583 - protein, 14 aa, synthetic construct
  REGION 1..14: note = Cell Penetrating Peptide / Homing Peptide / PMO Conjugates
  VARIANT 13: note = X = a neutral amino acid C(=O) (CHR)n NH-, where each R is independently H or methyl
  MOD_RES 5: note = X = beta alanine
  VARIANT 2: note = X = a neutral amino acid C(=O) (CHR)n NH-, where each R is independently H or methyl
  MOD_RES 11: note = X = beta alanine
  VARIANT 8: note = X = a neutral amino acid C(=O) (CHR)n NH-, where each R is independently H or methyl
  MOD_RES 14: note = X = beta alanine
  REPEAT 2: note = wherein (CHR) can be repeated between 2 to 7 times
  REPEAT 8: note = wherein (CHR) can be repeated between 2 to 7 times
  REPEAT 13: note = wherein (CHR) can be repeated between 2 to 7 times
RXRRXRRXRRXRXX

SEQ ID NO: 584 - DNA, 29 bp, synthetic construct
  misc_feature 1..29: note = Antisense sequence to target splice site of proprocessed human dystrophin
  source 1..29: mol_type = other DNA
ccactcagagctcagatcttctaacttcc

SEQ ID NO: 585 - DNA, 25 bp, synthetic construct
  misc_feature 1..25: note = Antisense sequence to target splice site of proprocessed human dystrophin
  source 1..25: mol_type = other DNA
gggatccagtatacttacaggctcc

SEQ ID NO: 586 - DNA, 27 bp, synthetic construct
  misc_feature 1..27: note = Antisense sequence to target splice site of proprocessed human dystrophin
  source 1..27: mol_type = other DNA
cttccactcagagctcagatcttctaa

SEQ ID NO: 587 - DNA, 30 bp, synthetic construct
  misc_feature 1..30: note = Antisense sequence to target splice site of proprocessed human dystrophin
  source 1..30: mol_type = other DNA
acatcaaggaagatggcatttctagtttgg

SEQ ID NO: 588 - DNA, 30 bp, synthetic construct
  misc_feature 1..30: note = Antisense sequence to target splice site of proprocessed human dystrophin
  source 1..30: mol_type = other DNA
ctccaacatcaaggaagatggcatttctag

SEQ ID NO: 589 - DNA, 24 bp, synthetic construct
  misc_feature 1..24: note = Antisense sequence to target splice site of proprocessed human dystrophin
  source 1..24: mol_type = other DNA
ttctgtccaagcccggttgaaatc

SEQ ID NO: 590 - DNA, 21 bp, synthetic construct
  misc_feature 1..21: note = Antisense sequence to target splice site of proprocessed human dystrophin
  source 1..21: mol_type = other DNA
cacccaccatcaccctcygtg

SEQ ID NO: 591 - DNA, 22 bp, synthetic construct
  misc_feature 1..22: note = Antisense sequence to target splice site of proprocessed human dystrophin
  source 1..22: mol_type = other DNA
atcatctcgttgatatcctcaa

SEQ ID NO: 592 - DNA, 25 bp, synthetic construct
  misc_feature 1..25: note = Antisense sequence to target splice site of proprocessed human dystrophin
  source 1..25: mol_type = other DNA
acatcaaggaagatggcatttctag

SEQ ID NO: 593 - DNA, 26 bp, synthetic construct
  misc_feature 1..26: note = Antisense sequence to target splice site of proprocessed human dystrophin
  source 1..26: mol_type = other DNA
accagagtaacagtctgagtaggagc

SEQ ID NO: 594 - DNA, 20 bp, synthetic construct
  misc_feature 1..20: note = Antisense sequence to target splice site of proprocessed human dystrophin
  source 1..20: mol_type = other DNA
tcaaggaagatggcatttct

SEQ ID NO: 595 - DNA, 23 bp, synthetic construct
  misc_feature 1..23: note = Antisense sequence to target splice site of proprocessed human dystrophin
  source 1..23: mol_type = other DNA
cctctgtgattttataacttgat

SEQ ID NO: 596 - DNA, 25 bp, synthetic construct
  misc_feature 1..25: note = Antisense sequence to target splice site of proprocessed human dystrophin
  source 1..25: mol_type = other DNA
atcattttttctcataccttctgct

SEQ ID NO: 597 - DNA, 23 bp, synthetic construct
  misc_feature 1..23: note = Antisense sequence to target splice site of proprocessed human dystrophin
  source 1..23: mol_type = other DNA
ctcataccttctgcttgatgatc

SEQ ID NO: 598 - DNA, 17 bp, synthetic construct
  misc_feature 1..17: note = Antisense sequence to target splice site of proprocessed human dystrophin
  source 1..17: mol_type = other DNA
tggcatttctagtttgg

SEQ ID NO: 599 - DNA, 23 bp, synthetic construct
  misc_feature 1..23: note = Antisense sequence to target splice site of proprocessed human dystrophin
  source 1..23: mol_type = other DNA
ccagagcaggtacctccaacatc

SEQ ID NO: 600 - DNA, 24 bp, synthetic construct
  misc_feature 1..24: note = Antisense sequence to target splice site of proprocessed human dystrophin
  source 1..24: mol_type = other DNA
tgttcagcttctgttagccactga

SEQ ID NO: 601 - DNA, 20 bp, synthetic construct
  misc_feature 1..20: note = Antisense sequence to target splice site of proprocessed human dystrophin
  source 1..20: mol_type = other DNA
tttgtgtctttctgagaaac

SEQ ID NO: 602 - DNA, 19 bp, synthetic construct
  misc_feature 1..19: note = Antisense sequence to target splice site of proprocessed human dystrophin
  source 1..19: mol_type = other DNA
cgccgccatttctcaacag

SEQ ID NO: 603 - DNA, 20 bp, synthetic construct
  misc_feature 1..20: note = Antisense sequence to target splice site of proprocessed human dystrophin
  source 1..20: mol_type = other DNA
atctgtcaaatcgcctgcag

SEQ ID NO: 604 - DNA, 20 bp, synthetic construct
  misc_feature 1..20: note = Antisense sequence to target splice site of proprocessed human dystrophin
  source 1..20: mol_type = other DNA
tgtttttgaggattgctgaa

SEQ ID NO: 605 - DNA, 19 bp, synthetic construct
  misc_feature 1..19: note = Antisense sequence to target splice site of proprocessed human dystrophin
  source 1..19: mol_type = other DNA
gctgaattatttcttcccc

SEQ ID NO: 606 - DNA, 17 bp, synthetic construct
  misc_feature 1..17: note = Antisense sequence to target splice site of proprocessed human dystrophin
  source 1..17: mol_type = other DNA
gcccaatgccatcctgg

SEQ ID NO: 607 - DNA, 26 bp, synthetic construct
  misc_feature 1..26: note = Antisense sequence to target splice site of proprocessed human dystrophin
  source 1..26: mol_type = other DNA
ccaatgccatcctggagttcctgtaa

SEQ ID NO: 608 - DNA, 31 bp, synthetic construct
  misc_feature 1..31: note = Antisense sequence to target splice site of proprocessed human dystrophin
  source 1..31: mol_type = other DNA
cattcaactgttgcctccggttctgaaggtg

SEQ ID NO: 609 - DNA, 25 bp, synthetic construct
  misc_feature 1..25: note = Antisense sequence to target splice site of proprocessed human dystrophin
  source 1..25: mol_type = other DNA
ctgaaggtgttcttgtacttcatcc

SEQ ID NO: 610 - DNA, 18 bp, synthetic construct
  misc_feature 1..18: note = Antisense sequence to target splice site of proprocessed human dystrophin
  source 1..18: mol_type = other DNA
ctgttgcctccggttctg

SEQ ID NO: 611 - DNA, 22 bp, synthetic construct
  misc_feature 1..22: note = Antisense sequence to target splice site of proprocessed human dystrophin
  source 1..22: mol_type = other DNA
attctttcaactagaataaaag

SEQ ID NO: 612 - DNA, 30 bp, synthetic construct
  misc_feature 1..30: note = Antisense sequence to target splice site of proprocessed human dystrophin
  source 1..30: mol_type = other DNA
gccatcctggagttcctgtaagataccaaa

SEQ ID NO: 613 - DNA, 30 bp, synthetic construct
  misc_feature 1..30: note = Antisense sequence to target splice site of proprocessed human dystrophin
  source 1..30: mol_type = other DNA
ccaatgccatcctggagttcctgtaagata

SEQ ID NO: 614 - DNA, 30 bp, synthetic construct
  misc_feature 1..30: note = Antisense sequence to target splice site of proprocessed human dystrophin
  source 1..30: mol_type = other DNA
gccgctgcccaatgccatcctggagttcct

SEQ ID NO: 615 - DNA, 30 bp, synthetic construct
  misc_feature 1..30: note = Antisense sequence to target splice site of proprocessed human dystrophin
  source 1..30: mol_type = other DNA
gtttgccgctgcccaatgccatcctggagt

SEQ ID NO: 616 - DNA, 30 bp, synthetic construct
  misc_feature 1..30: note = Antisense sequence to target splice site of proprocessed human dystrophin
  source 1..30: mol_type = other DNA
caacagtttgccgctgcccaatgccatcct

SEQ ID NO: 617 - DNA, 30 bp, synthetic construct
  misc_feature 1..30: note = Antisense sequence to target splice site of proprocessed human dystrophin
  source 1..30: mol_type = other DNA
ctgacaacagtttgccgctgcccaatgcca

SEQ ID NO: 618 - DNA, 30 bp, synthetic construct
  misc_feature 1..30: note = Antisense sequence to target splice site of proprocessed human dystrophin
  source 1..30: mol_type = other DNA
tgttctgacaacagtttgccgctgcccaat

SEQ ID NO: 619 - DNA, 30 bp, synthetic construct
  misc_feature 1..30: note = Antisense sequence to target splice site of proprocessed human dystrophin
  source 1..30: mol_type = other DNA
caatgttctgacaacagtttgccgctgccc

SEQ ID NO: 620 - DNA, 30 bp, synthetic construct
  misc_feature 1..30: note = Antisense sequence to target splice site of proprocessed human dystrophin
  source 1..30: mol_type = other DNA
cattcaatgttctgacaacagtttgccgct

SEQ ID NO: 621 - DNA, 30 bp, synthetic construct
  misc_feature 1..30: note = Antisense sequence to target splice site of proprocessed human dystrophin
  source 1..30: mol_type = other DNA
tatttcttccccagttgcattcaatgttct

SEQ ID NO: 622 - DNA, 30 bp, synthetic construct
  misc_feature 1..30: note = Antisense sequence to target splice site of proprocessed human dystrophin
  source 1..30: mol_type = other DNA
gctgaattatttcttccccagttgcattca

SEQ ID NO: 623 - DNA, 30 bp, synthetic construct
  misc_feature 1..30: note = Antisense sequence to target splice site of proprocessed human dystrophin
  source 1..30: mol_type = other DNA
ggattgctgaattatttcttccccagttgc

SEQ ID NO: 624 - DNA, 30 bp, synthetic construct
  misc_feature 1..30: note = Antisense sequence to target splice site of proprocessed human dystrophin
  source 1..30: mol_type = other DNA
tttgaggattgctgaattatttcttcccca

SEQ ID NO: 625 - DNA, 30 bp, synthetic construct
  misc_feature 1..30: note = Antisense sequence to target splice site of proprocessed human dystrophin
  source 1..30: mol_type = other DNA
gtacttcatcccactgattctgaattcttt

SEQ ID NO: 626 - DNA, 30 bp, synthetic construct
  misc_feature 1..30: note = Antisense sequence to target splice site of proprocessed human dystrophin
  source 1..30: mol_type = other DNA
tcttgtacttcatcccactgattctgaatt

SEQ ID NO: 627 - DNA, 30 bp, synthetic construct
  misc_feature 1..30: note = Antisense sequence to target splice site of proprocessed human dystrophin
  source 1..30: mol_type = other DNA
tgttcttgtacttcatcccactgattctga

SEQ ID NO: 628 - DNA, 30 bp, synthetic construct
  misc_feature 1..30: note = Antisense sequence to target splice site of proprocessed human dystrophin
  source 1..30: mol_type = other DNA
cggttctgaaggtgttcttgtacttcatcc

SEQ ID NO: 629 - DNA, 30 bp, synthetic construct
  misc_feature 1..30: note = Antisense sequence to target splice site of proprocessed human dystrophin
  source 1..30: mol_type = other DNA
ctccggttctgaaggtgttcttgtacttca

SEQ ID NO: 630 - DNA, 30 bp, synthetic construct
  misc_feature 1..30: note = Antisense sequence to target splice site of proprocessed human dystrophin
  source 1..30: mol_type = other DNA
tgcctccggttctgaaggtgttcttgtact

SEQ ID NO: 631 - DNA, 30 bp, synthetic construct
  misc_feature 1..30: note = Antisense sequence to target splice site of proprocessed human dystrophin
  source 1..30: mol_type = other DNA
tgttgcctccggttctgaaggtgttcttgt

SEQ ID NO: 632 - DNA, 30 bp, synthetic construct
  misc_feature 1..30: note = Antisense sequence to target splice site of proprocessed human dystrophin
  source 1..30: mol_type = other DNA
aactgttgcctccggttctgaaggtgttct

SEQ ID NO: 633 - DNA, 30 bp, synthetic construct
  misc_feature 1..30: note = Antisense sequence to target splice site of proprocessed human dystrophin
  source 1..30: mol_type = other DNA
ttcaactgttgcctccggttctgaaggtgt

SEQ ID NO: 634 - DNA, 25 bp, synthetic construct
  misc_feature 1..25: note = Antisense sequence to target splice site of proprocessed human dystrophin
  source 1..25: mol_type = other DNA
taaagctctggaaacctgaaaggaa

SEQ ID NO: 635 - DNA, 25 bp, synthetic construct
  misc_feature 1..25: note = Antisense sequence to target splice site of proprocessed human dystrophin
  source 1..25: mol_type = other DNA
ttcagcttctgtaagccaggcaaga

SEQ ID NO: 636 - DNA, 25 bp, synthetic construct
  misc_feature 1..25: note = Antisense oligomer
  source 1..25: mol_type = other DNA
ggccaaacctcggcttacctgaaat

SEQ ID NO: 637 - skipped

SEQ ID NO: 638 - protein, 12 aa, synthetic construct
  REGION 1..12: note = arginine-rich peptide transport
  VARIANT 2: note = X = a neutral amino acid C(=O) (CHR)n NH-, where each R is independently H or methyl
  VARIANT 6: note = X = a neutral amino acid C(=O) (CHR)n NH-, where each R is independently H or methyl
  VARIANT 8: note = X = a neutral amino acid C(=O) (CHR)n NH-, where each R is independently H or methyl
  VARIANT 12: note = X = a neutral amino acid C(=O) (CHR)n NH-, where each R is independently H or methyl
  REPEAT 2: note = wherein (CHR) can be repeated between 2 to 7 times
  REPEAT 6: note = wherein (CHR) can be repeated between 2 to 7 times
  REPEAT 8: note = wherein (CHR) can be repeated between 2 to 7 times
  REPEAT 12: note = wherein (CHR) can be repeated between 2 to 7 times
RXRRRXRXRRRX

SEQ ID NO: 639 - protein, 9 aa, synthetic construct
  REGION 1..9: note = arginine-rich peptide transport
  VARIANT 3: note = X = a neutral amino acid C(=O) (CHR)n NH-, where each R is independently H or methyl
  VARIANT 5: note = X = a neutral amino acid C(=O) (CHR)n NH-, where each R is independently H or methyl
  VARIANT 9: note = X = a neutral amino acid C(=O) (CHR)n NH-, where each R is independently H or methyl
  REPEAT 3: note = wherein (CHR) can be repeated between 2 to 7 times
  REPEAT 5: note = wherein (CHR) can be repeated between 2 to 7 times
  REPEAT 9: note = wherein (CHR) can be repeated between 2 to 7 times
RRXRXRRRX

SEQ ID NO: 640 - DNA, 24 bp, synthetic construct
  misc_feature 1..24: note = PCR Primer
  source 1..24: mol_type = other DNA
ccagagctttacctgagaaacaag

SEQ ID NO: 641 - DNA, 21 bp, synthetic construct
  misc_feature 1..21: note = PCR Primer
  source 1..21: mol_type = other DNA
ccagccactcagccagtgaag

SEQ ID NO: 642 - DNA, 24 bp, synthetic construct
  misc_feature 1..24: note = PCR Primer
  source 1..24: mol_type = other DNA
cgatccgtaatgattgttctagcc

SEQ ID NO: 643 - DNA, 24 bp, synthetic construct
  misc_feature 1..24: note = PCR Primer
  source 1..24: mol_type = other DNA
catttcattcaactgttgcctccg

SEQ ID NO: 644 - DNA, 21 bp, synthetic construct
  misc_feature 1..21: note = PCR Primer
  source 1..21: mol_type = other DNA
caatgctcctgacctctgtgc

SEQ ID NO: 645 - DNA, 22 bp, synthetic construct
  misc_feature 1..22: note = PCR Primer
  source 1..22: mol_type = other DNA
gtctacaacaaagctcaggtcg

SEQ ID NO: 646 - DNA, 25 bp, synthetic construct
  misc_feature 1..25: note = PCR Primer
  source 1..25: mol_type = other DNA
gcaatgttatctgcttcctccaacc

SEQ ID NO: 647 - DNA, 22 bp, synthetic construct
  misc_feature 1..22: note = PCR Primer
  source 1..22: mol_type = other DNA
gctcttttccaggttcaagtgg

SEQ ID NO: 648 - DNA, 23 bp, synthetic construct
  misc_feature 1..23: note = PCR Primer
  source 1..23: mol_type = other DNA
cttggacagaacttaccgactgg

SEQ ID NO: 649 - DNA, 21 bp, synthetic construct
  misc_feature 1..21: note = PCR Primer
  source 1..21: mol_type = other DNA
gcaggatttggaacagaggcg

SEQ ID NO: 650 - DNA, 23 bp, synthetic construct
  misc_feature 1..23: note = PCR Primer
  source 1..23: mol_type = other DNA
catctacatttgtctgccactgg

SEQ ID NO: 651 - DNA, 23 bp, synthetic construct
  misc_feature 1..23: note = PCR Primer
  source 1..23: mol_type = other DNA
gtttcttccaaagcagcctctcg

(19) United States
(12) Patent Application Publication
(10) Pub. No.: US 2025/0154507 A1
(43) Pub. Date: May 15, 2025

(54) MULTIPLE EXON SKIPPING COMPOSITIONS FOR DMD

(71) Applicant: Sarepta Therapeutics, Inc., Cambridge, MA (US)

(72) Inventors: Peter SAZANI, Bothell, WA (US); Ryszard KOLE, Bellevue, WA (US)

(73) Assignee: Sarepta Therapeutics, Inc., Cambridge, MA (US)

(21) Appl. No.: 18/778,132

(22) Filed: Jul. 19, 2024

Related U.S. Application Data
(63) Continuation of application No. 18/499,448, filed on Nov. 1, 2023, now abandoned.
Application No. 18/499,448 is a continuation of application No. 18/150,352, filed on Jan. 5, 2023, now abandoned.
Application No. 18/150,352 is a continuation of application No. 16/899,802, filed on Jun. 12, 2020, now abandoned.
Application No. 16/899,802 is a continuation of application No. 16/243,926, filed on Jan. 9, 2019, now abandoned.
Application No. 16/243,926 is a continuation of application No. 16/146,328, filed on Sep. 28, 2018, now abandoned.
Application No. 16/146,328 is a continuation of application No. 15/789,862, filed on Oct. 20, 2017, now abandoned.
Application No. 15/789,862 is a continuation of application No. 15/349,778, filed on Nov. 11, 2016, now abandoned.
Application No. 15/349,778 is a continuation of application No. 14/523,610, filed on Oct. 24, 2014, now abandoned.
Application No. 14/523,610 is a division of application No. 12/605,276, filed on Oct. 23, 2009, now Pat. No. 8,871,918.
(60) Provisional application No. 61/108,416, filed on Oct. 24, 2008.

(51) Int. Cl. C12N 15/113; C12N 15/11

(52) U.S. Cl. CPC C12N 15/113; C12N 15/111; C12N 2310/11; C12N 2310/321; C12N 2310/3233; C12N 2310/331; C12N 2310/3341; C12N 2310/3513; C12N 2320/30; C12N 2320/33

(57) ABSTRACT

Provided are antisense molecules capable of binding to a selected target site in the human dystrophin gene to induce exon skipping, and methods of use thereof to treat muscular dystrophy.

### CROSS-REFERENCE TO RELATED APPLICATION

This application claims the benefit under 35 U.S.C. § 119(e) of U.S. Provisional Patent Application No. 61/108,416 filed Oct. 24, 2008; wherein this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 120178_410_SEQUENCE_LISTING.txt. The text file is 157 KB, was created on Oct. 23, 2009 and is being submitted electronically via EFS-Web.

### FIELD OF THE INVENTION

The present invention relates to novel antisense compounds and compositions suitable for facilitating exon skipping in the human dystrophin gene. It also provides methods for inducing exon skipping using the antisense compositions adapted for use in the methods of the invention.

### BACKGROUND OF THE INVENTION

Antisense technologies are being developed using a range of chemistries to affect gene expression at a variety of different levels (transcription, splicing, stability, translation). Much of that research has focused on the use of antisense compounds to correct or compensate for abnormal or disease-associated genes in a wide range of indications. Antisense molecules are able to inhibit gene expression with specificity, and because of this, many research efforts concerning oligonucleotides as modulators of gene expression have focused on inhibiting the expression of targeted genes or the function of cis-acting elements. The antisense oligonucleotides are typically directed against RNA, either the sense strand (e.g., mRNA) or minus-strand in the case of some viral RNA targets. To achieve a desired effect of specific gene down-regulation, the oligonucleotides generally either promote the decay of the targeted mRNA, block translation of the mRNA or block the function of cis-acting RNA elements thereby effectively preventing either de novo synthesis of the target protein or replication of the viral RNA.

However, such techniques are not useful where the object is to up-regulate production of the native protein or compensate for mutations that induce premature termination of translation such as nonsense or frame-shifting mutations. In these cases, the defective gene transcript should not be subjected to targeted degradation or steric inhibition, so the antisense oligonucleotide chemistry should not promote target mRNA decay or block translation.

In a variety of genetic diseases, the effects of mutations on the eventual expression of a gene can be modulated through a process of targeted exon skipping during the splicing process. The splicing process is directed by complex multi-component machinery that brings adjacent exon-intron junctions in pre-mRNA into close proximity and performs cleavage of phosphodiester bonds at the ends of the introns with their subsequent reformation between exons that are to be spliced together. This complex and highly precise process is mediated by sequence motifs in the pre-mRNA that are relatively short semi-conserved RNA segments to which bind the various nuclear splicing factors that are then involved in the splicing reactions. By changing the way the splicing machinery reads or recognizes the motifs involved in pre-mRNA processing, it is possible to create differentially spliced mRNA molecules. It has now been recognized that the majority of human genes are alternatively spliced during normal gene expression, although the mechanisms involved have not been identified.

In cases where a normally functional protein is prematurely terminated because of mutations therein, a means for restoring some functional protein production through antisense technology has been shown to be possible through intervention during the splicing processes, and that if exons associated with disease-causing mutations can be specifically deleted from some genes, a shortened protein product can sometimes be produced that has similar biological properties of the native protein or has sufficient biological activity to ameliorate the disease caused by mutations associated with the exon (Sierakowska, Sambade et al. 1996; Wilton, Lloyd et al. 1999; van Deutekom, Bremmer-Bout et al. 2001; Lu, Mann et al. 2003; Aartsma-Rus, Janson et al. 2004). Kole et al. (U.S. Pat. Nos. 5,627,274; 5,916,808; 5,976,879; and 5,665,593) disclose methods of combating aberrant splicing using modified antisense oligonucleotide analogs that do not promote decay of the targeted pre-mRNA. Bennett et al (U.S. Pat. No. 6,210,892) describe antisense modulation of wild-type cellular mRNA processing also using antisense oligonucleotide analogs that do not induce RNAse H-mediated cleavage of the target RNA.

The process of targeted exon skipping is likely to be particularly useful in long genes where there are many exons and introns, where there is redundancy in the genetic constitution of the exons or where a protein is able to function without one or more particular exons. Efforts to redirect gene processing for the treatment of genetic diseases associated with truncations caused by mutations in various genes have focused on the use of antisense oligonucleotides that either: (1) fully or partially overlap with the elements involved in the splicing process; or (2) bind to the pre-mRNA at a position sufficiently close to the element to disrupt the binding and function of the splicing factors that would normally mediate a particular splicing reaction which occurs at that element.

Duchenne muscular dystrophy (DMD) is caused by a defect in the expression of the protein dystrophin. The gene encoding the protein contains 79 exons spread out over more than 2 million nucleotides of DNA. Any exonic mutation that changes the reading frame of the exon, or introduces a stop codon, or is characterized by removal of an entire out of frame exon or exons or duplications of one or more exons has the potential to disrupt production of functional dystrophin, resulting in DMD.

A less severe form of muscular dystrophy, Becker muscular dystrophy (BMD) has been found to arise where a mutation, typically a deletion of one or more exons, results in a correct reading frame along the entire dystrophin transcript, such that translation of mRNA into protein is not prematurely terminated. If the joining of the upstream and downstream exons in the processing of a mutated dystrophin pre-mRNA maintains the correct reading frame of the gene, the result is an mRNA coding for a protein with a short internal deletion that retains some activity resulting in a Becker phenotype.

Deletions of an exon or exons which do not alter the reading frame of a dystrophin protein give rise to a BMD phenotype, whereas an exon deletion that causes a frame-shift will give rise to DMD (Monaco, Bertelson et al. 1988). In general, dystrophin mutations including point mutations and exon deletions that change the reading frame and thus interrupt proper protein translation result in DMD. It should also be noted that some BMD and DMD patients have exon deletions covering multiple exons.

Although antisense molecules may provide a tool in the treatment of Duchenne Muscular Dystrophy (DMD), attempts to induce exon skipping using antisense molecules have had mixed success. Successful skipping of dystrophin exon 19 from the dystrophin pre-mRNA was achieved using a variety of antisense molecules directed at the flanking splice sites or motifs within the exon involved in exon definition as described by Errington et al., (Errington, Mann et al. 2003).

The first example of specific and reproducible exon skipping in the mdx mouse model was reported by Wilton et al (Wilton, Lloyd et al. 1999). By directing an antisense molecule to the donor splice site, exon 23 skipping was induced in the dystrophin mRNA within 6 hours of treatment of the cultured cells. Wilton et al also describe targeting the acceptor region of the mouse dystrophin pre-mRNA with longer antisense oligonucleotides. While the first antisense oligonucleotide directed at the intron 23 donor splice site induced exon skipping in primary cultured myoblasts, this compound was found to be much less efficient in immortalized cell cultures expressing higher levels of dystrophin.

Despite these efforts, there remains a need for improved antisense oligomers targeted to multiple dystrophin exons and improved muscle delivery compositions and methods for DMD therapeutic applications.

### BRIEF SUMMARY OF THE INVENTION

Embodiments of the present invention relate generally to antisense compounds capable of binding to a selected target to induce exon skipping, and methods of use thereof to induce exon skipping. In certain embodiments, it is possible to combine two or more antisense oligonucleotides of the present invention together to induce single or multiple exon skipping.

In certain embodiments, it is possible to improve exon skipping of a single or multiple exons by covalently linking together two or more antisense oligonucleotide molecules (see, e.g., Aartsma-Rus, Janson et al. 2004).

In certain embodiments, the antisense compounds of the present invention induce exon skipping in the human dystrophin gene, and thereby allow muscle cells to produce a functional dystrophin protein.

The antisense oligonucleotide compounds (also referred to herein as oligomers) of the present invention typically: (i) comprise morpholino subunits and phosphorus-containing intersubunit linkages joining a morpholino nitrogen of one subunit to a 5′ exocyclic carbon of an adjacent subunit, (ii) contain between 10-40 nucleotide bases, preferably 20-35 bases (ili) comprise a base sequence effective to hybridize to at least 12 contiguous bases of a target sequence in dystrophin pre-mRNA and induce exon skipping.

In certain embodiments, the antisense compounds of the present invention may comprise phosphorus-containing intersubunit linkages joining a morpholino nitrogen of one subunit to a 5′ exocyclic carbon of an adjacent subunit, in accordance with the following structure (I):

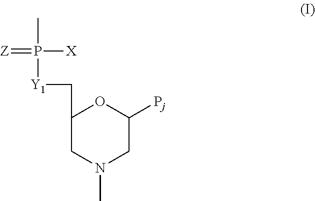

wherein:

Y₁ is —O—, —S—, —NH—, or —CH₂—;

Z is O or S;

Pj is a purine or pyrimidine base-pairing moiety effective to bind, by base-specific hydrogen bonding, to a base in a polynucleotide; and

X is fluoro, optionally substituted alkyl, optionally substituted alkoxy, optionally substituted thioalkoxy, amino, optionally substituted alkylamino, or optionally substituted heterocyclyl.

In certain embodiments, the above intersubunit linkages, which are uncharged, may be interspersed with linkages that are positively charged at physiological pH, where the total number of positively charged linkages is between 2 and no more than half of the total number of linkages. For example, the positively charged linkages may have the above structure in which X is optionally substituted 1-piperazinyl. In other embodiments, the positively charged linkages may have the above structure in which X is substituted 1-piperazynyl, wherein the 1-piperazynyl is substituted at the 4-position with an optionally substituted alkyl guanidynyl moiety.

Where the antisense compound administered is effective to target a splice site of preprocessed human dystrophin, it may have a base sequence complementary to a target region containing at least 12 contiguous bases in a preprocessed messenger RNA (mRNA) human dystrophin transcript. Exemplary antisense sequences include those identified by SEQ ID NOS: 1 to 569 and 612 to 633.

In certain embodiments, an antisense sequence of the present invention is contained within:

(a) any of the sequences identified by SEQ ID NOS: 1-20, preferably SEQ ID NOS: 4, 8. 11 and 12, and more preferably SEQ ID NO: 12 for use in producing skipping of exon 44 in the processing of human dystrophin pre-processed mRNA;

(b) any of the sequences identified by SEQ ID NOS: 21-76 and 612 to 624, preferably SEQ ID NOS: 27, 29, 34 and 39, and more preferably SEQ ID NO: 34 for use in producing skipping of exon 45 in the processing of human dystrophin pre-processed mRNA;

(c) any of the sequences identified by SEQ ID NOS: 77-125, preferably SEQ ID NOS: 21 to 53, and more preferably SEQ ID NOS: 82, 84-87, 90 96, 98, 99 and 101, for use in producing skipping of exon 46 in the processing of human dystrophin pre-processed mRNA;

(d) any of the sequences identified by SEQ ID NOS: 126-169,preferably SEQ ID NOS: 126-149, and more preferably SEQ ID NOS: 126, 128-130, 132, 144 and 146-149, for use in producing skipping of exon 47 in the processing of human dystrophin pre-processed mRNA;

(e) any of the sequences identified by SEQ ID NOS: 170-224 and 634, preferably SEQ ID NOS: 170-201 and 634, and more preferably SEQ ID NOS: 176, 178, 181-183, 194 and 198-201, for use in producing skipping of exon 48 in the processing of human dystrophin pre-processed mRNA;

(f) any of the sequences identified by SEQ ID NOS: 225-266, preferably SEQ ID NOS: 225-248, and more preferably SEQ ID NOS: 227, 229, 234, 236, 237 and 244-248, for use in producing skipping of exon 49 in the processing of human dystrophin pre-processed mRNA;

(g) any of the sequences identified by SEQ ID NOS: 267-308, preferably SEQ ID NOS: 277, 287 and 290, and more preferably SEQ ID NO: 287, for use in producing skipping of exon 50 in the processing of human dystrophin pre-processed mRNA;

(h) any of the sequences identified by SEQ ID NOS: 309-371, preferably SEQ ID NOS: 324, 326 and 327, and more preferably SEQ ID NO: 327 for use in producing skipping of exon 51 in the processing of human dystrophin pre-processed mRNA;

(i) any of the sequences identified by SEQ ID NOS: 372-415, preferably SEQ ID NOS: 372-397, and more preferably SEQ ID NOS: 379-382, 384, 390 and 392-395 for use in producing skipping of exon 52 in the processing of human dystrophin pre-processed mRNA;

(j) any of the sequences identified by SEQ ID NOS: 416-475 and 625-633, preferably SEQ ID NOS: 428, 429 and 431, and more preferably SEQ ID NO: 429, for use in producing skipping of exon 53 in the processing of human dystrophin pre-processed mRNA;

(k) any of the sequences identified by SEQ ID NOS: 476-519, preferably SEQ ID NOS: 476-499, and more preferably SEQ ID NOS: 479-482, 484, 489 and 491-493, for use in producing skipping of exon 54 in the processing of human dystrophin pre-processed mRNA; and

(l) any of the sequences identified by SEQ ID NOS: 520-569 and 635, preferably SEQ ID NOS: 520-546 and 635, and more preferably SEQ ID NOS: 524-528, 537, 539, 540, 542 and 544, for use in producing skipping of exon 55 in the processing of human dystrophin pre-processed mRNA;

In certain embodiments, the compound may be conjugated to an arginine-rich polypeptide effective to promote uptake of the compound into cells. Exemplary peptides include those identified by SEQ ID NOS: 570 to 578, among others described herein.

In one exemplary embodiment, the arginine-rich polypeptide is covalently coupled at its N-terminal or C-terminal residue to the 3′ or 5′ end of the antisense compound. Also in an exemplary embodiment, the antisense compound is composed of morpholino subunits and phosphorus-containing intersubunit linkages joining a morpholino nitrogen of one subunit to a 5′ exocyclic carbon of an adjacent subunit.

In general, the peptide-oligomer conjugate may further comprise a homing peptide which is selective for a selected mammalian tissue, i.e., the same tissue being targeted by the cell-penetrating peptide. The conjugate may be of the form: cell penetrating peptide—homing peptide—antisense oligomer, or, more preferably, of the form: homing peptide—cell penetrating peptide—antisense oligomer. For example, a peptide conjugate compound for use in treating Duchenne muscular dystrophy, as described above, can further comprise a homing peptide which is selective for muscle tissue, such as the peptide having the sequence identified as SEQ ID NO: 579, conjugated to the cell-penetrating peptide. Exemplary conjugates of this type include those represented herein as CP06062-MSP-PMO (cell penetrating peptide—homing peptide—antisense oligomer) and as MSP-CP06062-PMO (homing peptide—cell penetrating peptide—antisense oligomer) (see SEQ ID NOs: 580-583).

In some embodiments, the peptide is conjugated to the oligomer via a linker moiety. In certain embodiments the linker moiety may comprise an optionally substituted piperazynyl moiety. In other embodiments, the linker moiety may further comprise a beta alanine and/or a 6-aminohexanoic acid subunit. In yet other embodiments, the peptide is conjugated directly to the oligomer without a linker moiety.

Conjugation of the peptide to the oligomer may be at any position suitable for forming a covalent bond between the peptide and the oligomer or between the linker moiety and the oligomer. For example, in some embodiments conjugation of the peptide may be at the 3′ end of the oligomer. In other embodiments, conjugation of the peptide to the oligomer may be at the 5′ end of the oligomer. In yet other embodiments, the peptide may be conjugated to the oligomer through any of the intersubunit linkages.

In some embodiments, the peptide is conjugated to the oligomer at the 5′ end of the oligomer. In embodiments comprising phosphorus-containing intersubunit linkages, the peptide may be conjugated to the oligomer via a covalent bond to the phosphorous of the terminal linkage group. Conjugation in this manner may be with or without the linker moiety described above.

In yet other embodiments, the peptide may be conjugated to the oligomer at the 3′ end of the oligomer. In some further embodiments, the peptide may be conjugated to the nitrogen atom of the 3′ terminal morpolino group of the oligomer. In this respect, the peptide may be conjugated to the oligomer directly or via the linker moiety described above.

In some embodiments, the oligomer may be conjugated to a moiety that enhances the solubility of the oligomer in aqueous medium. In some embodiments, the moiety that enhances solubility of the oligomer in aqueous medium is a polyethyleneglycol. In yet further embodiments, the moiety that enhances solubility of the oligomer in aqueous medium is triethylene glycol. For example, in some embodiments the moiety that enhances solubility in aqueous medium may be conjugated to the oligomer at the 5′ end of the oligomer. Conjugation of the moiety that enhances solubility of the oligomer in aqueous medium to the oligomer may be either directly or through the linker moiety described above.

Certain embodiments of the present invention provide antisense molecules selected and or adapted to aid in the prophylactic or therapeutic treatment of a genetic disorder comprising at least an antisense molecule in a form suitable for delivery to a patient.

Certain embodiments of the invention provide methods for treating a patient suffering from a genetic disease wherein there is a mutation in a gene encoding a particular protein and the affect of the mutation can be abrogated by exon skipping, comprising the steps of: (a) selecting an antisense molecule in accordance with the methods described herein; and (b) administering the molecule to a patient in need of such treatment. The present invention also includes the use of purified and isolated antisense oligonucleotides of the invention, for the manufacture of a medicament for treatment of a genetic disease.

Certain embodiments provide a method of treating muscular dystrophy, such as a condition characterized by Duchenne muscular dystrophy, which method comprises administering to a patient in need of treatment an effective amount of an appropriately designed antisense oligonucleotide, as described herein, relevant to the particular genetic lesion in that patient. Further, certain embodiments provide a method for prophylactically treating a patient to prevent or at least minimize muscular dystrophy, including Duchene muscular dystrophy, comprising the step of: administering to the patient an effective amount of an antisense oligonucleotide or a pharmaceutical composition comprising one or more of these biological molecules.

Certain embodiments relate to methods of treating muscular dystrophy in a subject, comprising administering to the subject an effective amount of a substantially uncharged antisense compound containing 20-35 morpholino subunits linked by phosphorus-containing intersubunit linkages joining a morpholino nitrogen of one subunit to a 5′ exocyclic carbon of an adjacent subunit, comprising a sequence selected from the group consisting SEQ ID NOS: 1 to 569 and 612 to 635, and capable of forming with the complementary mRNA sequence in a dystrophin-gene exon a heteroduplex structure between said compound and mRNA having a Tm of at least 45° C., wherein the exon is selected from the group consisting of exons 44-55.

In certain embodiments, the muscular dystrophy is Duchenne's muscular dystrophy (DMD). In certain embodiments, the muscular dystrophy is Becker muscular dystrophy (BMD).

In certain embodiments, the sequence is selected from the group consisting SEQ ID NOS: 1-20, and the exon is exon 44. In certain embodiments, the sequence is selected from the group consisting SEQ ID NOS: 21-76 and 612 to 624, and the exon is exon 45.

In certain embodiments, the sequence is selected from the group consisting SEQ ID NOS: 77-125, and the exon is exon 46. In certain embodiments, the sequence selected from the group consisting SEQ ID NOS: 126-169, and the exon is exon 47.

In certain embodiments, the sequence is selected from the group consisting SEQ ID NOS: 170-224 and 634, and the exon is exon 48. In certain embodiments, the sequence selected from the group consisting SEQ ID NOS: 225-266, and the exon is exon 49.

In certain embodiments, the sequence is selected from the group consisting SEQ ID NOS: 267-308, and the exon is exon 50. In certain embodiments, the sequence is selected from the group consisting SEQ ID NOS: 309-371, and the exon is exon 51.

In certain embodiments, the sequence is selected from the group consisting SEQ ID NOS: 372-415, and the exon is exon 52. In certain embodiments, the sequence is selected from the group consisting SEQ ID NOS: 416-475 and 625-633, and the exon is exon 53. In certain embodiments, the sequence is selected from the group consisting SEQ ID NOS: 476-519, and the exon is exon 54. In certain embodiments, the sequence is selected from the group consisting SEQ ID NOS: 520-569 and 635, and the exon is exon 55. In certain embodiments, the sequence comprises or consists essentially of SEQ ID NO:287.

Certain embodiments provide kits for treating a genetic disease, which kits comprise at least an antisense oligonucleotide of the present invention, packaged in a suitable container and instructions for its use.

These and other objects and features will be more fully understood when the following detailed description of the invention is read in conjunction with the figures.

### BRIEF DESCRIPTION OF THE FIGURES

FIG. 1A shows an exemplary morpholino oligomer structure with a phosphorodiamidate linkage;

FIG. 1B shows a conjugate of an arginine-rich peptide and an antisense oligomer, in accordance with an embodiment of the invention;

FIG. 1C shows a conjugate as in FIG. 1B, wherein the backbone linkages contain one or more positively charged groups;

FIGS. 1D-G show the repeating subunit segment of exemplary morpholino oligonucleotides, designated D through G.

FIG. 2A shows the relative location and results of an antisense oligomer exon 51 scan designed to induce skipping of human dystrophin exon 51.

FIG. 2B-C shows the relative activity in cultured human rhabdomyosarcoma (RD) cells and human primary skeletal muscle cells of the three best oligomers selected from the exon 51 scan (SEQ ID NOs: 324, 326 and 327) relative to sequences (AVI-5658; SEQ ID NO: 588 and h51AON1; SEQ ID NO: 594) that are effective at inducing exon 51 skipping. FIG. 2D shows the relative location within exon 51 of three selected oligomers compared to certain sequences.

FIG. 3A shows the relative location and results of an antisense oligomer exon 50 scan designed to induce skipping of human dystrophin exon 50 compared to other sequences that induce exon 50 skipping.

FIG. 3B shows the relative location and activity of antisense sequences selected from the exon 50 scan (SEQ ID NOS: 277, 287, 290 and 291) compared to other sequences (SEQ ID NOS: 584 and 585).

FIG. 4A shows the relative location and results of an antisense oligomer exon 53 scan designed to induce skipping of human dystrophin exon 53. FIG. 4B shows the relative location of certain sequences used to compare the exon-skipping activity of those oligomers selected as being most active in the exon 53 scan.

FIGS. 4C-F show the results of dose-ranging studies, summarized in FIG. 4G, using the oligomers selected as being most efficacious in the exon 53 scan (SEQ ID NOS: 422, 428, 429 and 431).

FIGS. 4H and 4I show the relative activity of certain sequences (SEQ ID NOS: 608-611) compared to the activity of the most active exon 53-skipping oligomer (SEQ ID NO:429) in both RD cells and human primary skeletal muscle cells.

FIG. 5A shows the relative location and results of an antisense oligomer exon 44 scan designed to induce skipping of human dystrophin exon 44. FIG. 5B shows the relative location within exon 44 of certain sequences used to compare the exon-skipping activity to those oligomers selected as being most active in the exon 44 scan.

FIGS. 5C-G show the results of dose-ranging studies, summarized in FIG. 5H, using the oligomers selected as being most efficacious in the exon 44 scan (SEQ ID NOS: 4, 8, 11, 12 and 13).

FIGS. 5I and 5J show the relative activity of certain sequences (SEQ ID NOS: 600-603) compared to the activity of the most active exon 53-skipping oligomer (SEQ ID NO:12) in both RD cells and human primary skeletal muscle cells.

FIG. 6A shows the relative location and results of an antisense oligomer exon 45 scan designed to induce skipping of human dystrophin exon 45. FIG. 6B shows the relative location within exon 45 of certain sequences used to compare the exon-skipping activity to those oligomers selected as being most active in the exon 45 scan.

FIGS. 6C-F show the results of dose-ranging studies, summarized in FIG. 6H, using the oligomers selected as being most efficacious in the exon 45 scan (SEQ ID NOS: 27, 29, 34 and 39). FIG. 6G uses a relatively inactive oligomer (SEQ ID NO: 49) as a negative control.

FIGS. 6I and 6J show the relative activity of certain sequences (SEQ ID NOS: 604-607) compared to the activity of the most active exon 53-skipping oligomer (SEQ ID NO: 34) in both RD cells and human primary skeletal muscle cells.

### DETAILED DESCRIPTION OF THE INVENTION

Embodiments of the present invention relate generally to improved antisense compounds, and methods of use thereof, which are specifically designed to induce exon skipping in the dystrophin gene. Dystrophin plays a vital role in muscle function, and various muscle-related diseases are characterized by mutated forms of this gene. Hence, in certain embodiments, the improved antisense compounds described herein induce exon skipping in mutated forms of the human dystrophin gene, such as the mutated dystrophin genes found in Duchenne's muscular dystrophy (DMD) and Becker's muscular dystrophy (BMD).

Due to aberrant mRNA splicing events caused by mutations, these mutated human dystrophin genes either express defective dystrophin protein or express no measurable dystrophin at all, a condition that leads to various forms of muscular dystrophy. To remedy this condition, the antisense compounds of the present invention typically hybridize to selected regions of a pre-processed RNA of a mutated human dystrophin gene, induce exon skipping and differential splicing in that otherwise aberrantly spliced dystrophin mRNA, and thereby allow muscle cells to produce an mRNA transcript that encodes a functional dystrophin protein. In certain embodiments, the resulting dystrophin protein is not necessarily the “wild-type” form of dystrophin, but is rather a truncated, yet functional or semi-functional, form of dystrophin.

By increasing the levels of functional dystrophin protein in muscle cells, these and related embodiments may be useful in the prophylaxis and treatment of muscular dystrophy, especially those forms of muscular dystrophy, such as DMD and BMD, that are characterized by the expression of defective dystrophin proteins due to aberrant mRNA splicing. The specific oligomers described herein further provide improved, dystrophin-exon-specific targeting over other oligomers in use, and thereby offer significant and practical advantages over alternate methods of treating relevant forms of muscular dystrophy.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

### Definitions

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus.

The terms “cell penetrating peptide” or “CPP” are used interchangeably and refer to cationic cell penetrating peptides, also called transport peptides, carrier peptides, or peptide transduction domains. The peptides, as shown herein, have the capability of inducing cell penetration within 30%, 40%, 50%, 60%, 70%, 80%, 90%, or 100% of cells of a given cell culture population, including all integers in between, and allow macromolecular translocation within multiple tissues in vivo upon systemic administration.

The terms “antisense oligomer” or “antisense compound” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence. The cyclic subunits are based on ribose or another pentose sugar or, in a preferred embodiment, a morpholino group (see description of morpholino oligomers below).

Such an antisense oligomer can be designed to block or inhibit translation of mRNA or to inhibit natural pre-mRNA splice processing, and may be said to be “directed to” or “targeted against” a target sequence with which it hybridizes. In certain embodiments, the target sequence includes a region including an AUG start codon of an mRNA, a 3′ or 5′ splice site of a pre-processed mRNA, or a branch point. The target sequence may be within an exon or within an intron. The target sequence for a splice site may include an mRNA sequence having its 5′ end 1 to about 25 base pairs downstream of a normal splice acceptor junction in a preprocessed mRNA. A preferred target sequence for a splice is any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above. Included are antisense oligomers that comprise, consist essentially of, or consist of one or more of SEQ ID NOS: 1 to 569 and 612 to 635. Also included are variants of these antisense oligomers, including variant oligomers having 80%, 85%, 90%, 95%, 97%, 98%, or 99% (including all integers in between) sequence identity or sequence homology to any one of SEQ ID NOS: 1 to 569 and 612 to 635, and/or variants that differ from these sequences by about 1, 2, 3, 4, 5, 6, 7, 8, 9, or 10 nucleotides, preferably those variants that induce exon skipping of one or more selected human dystrophin exons. Also included are oligomers of any on or more of SEQ ID NOS: 584-611 and 634-635, which comprise a suitable number of charged linkages, as described herein, e.g. up to about 1 per every 2-5 uncharged linkages, such as about 4-5 per every 10 uncharged linkages, and/or which comprise an Arg-rich peptide attached thereto, as also described herein.

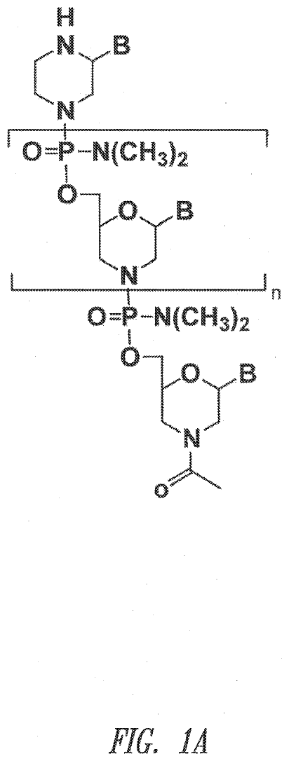

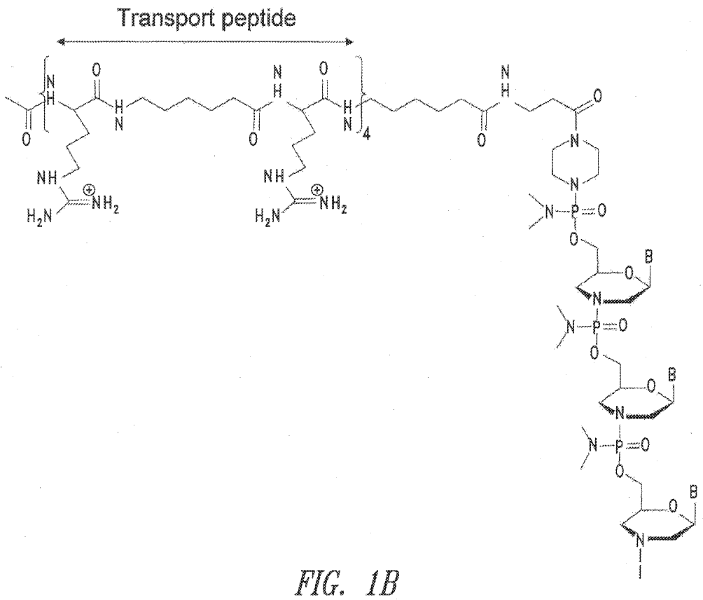

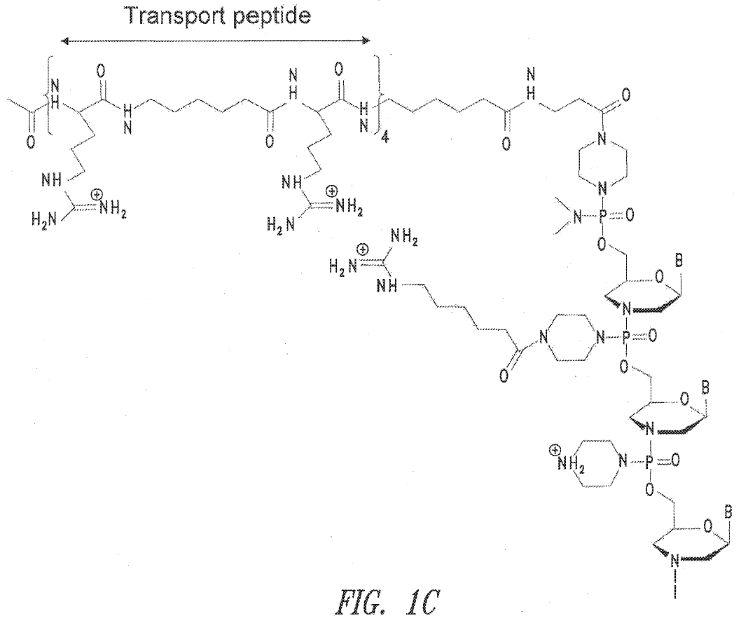

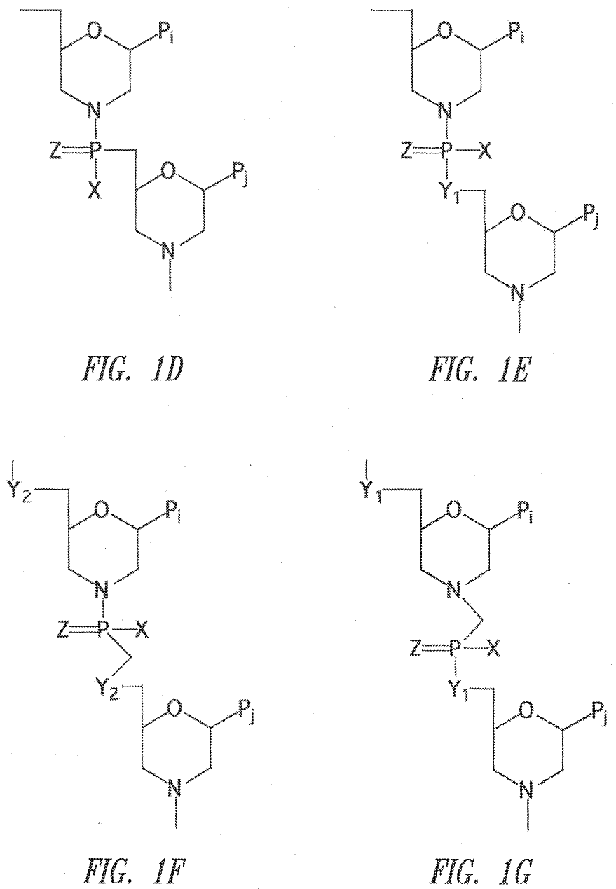

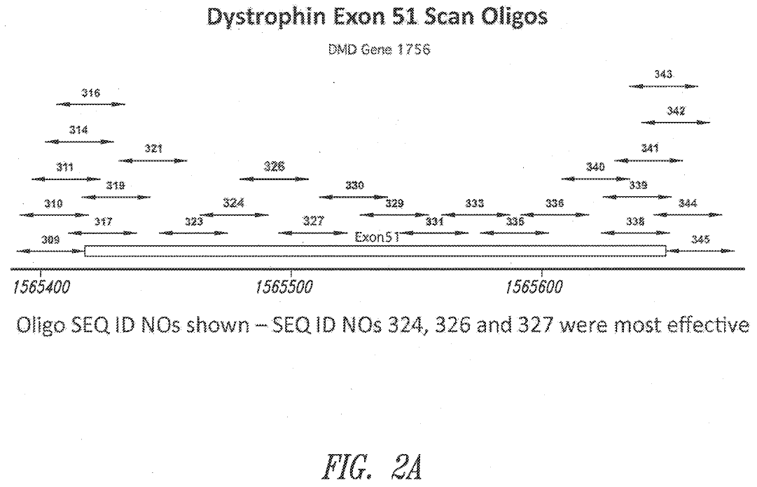

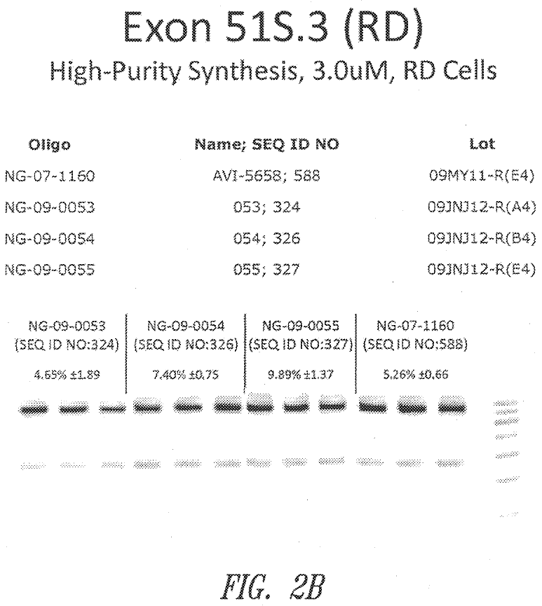

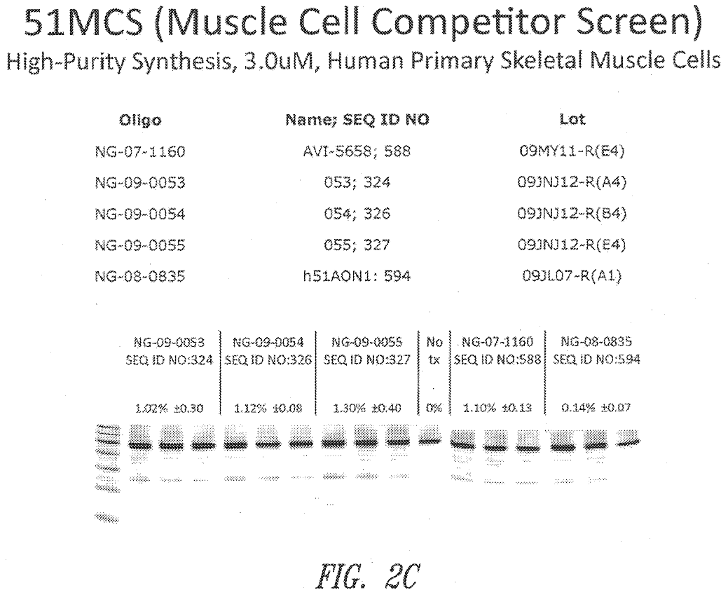

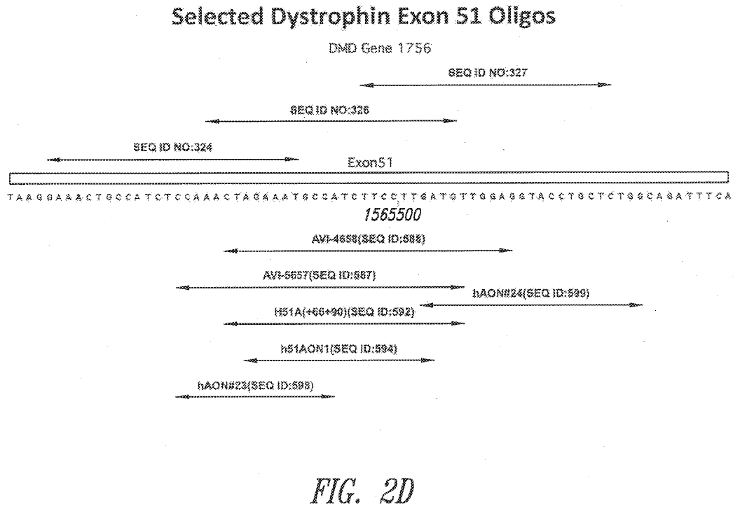

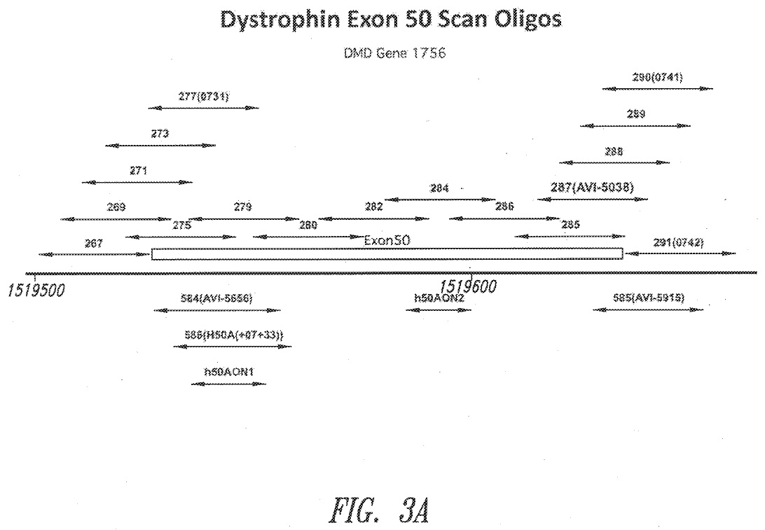

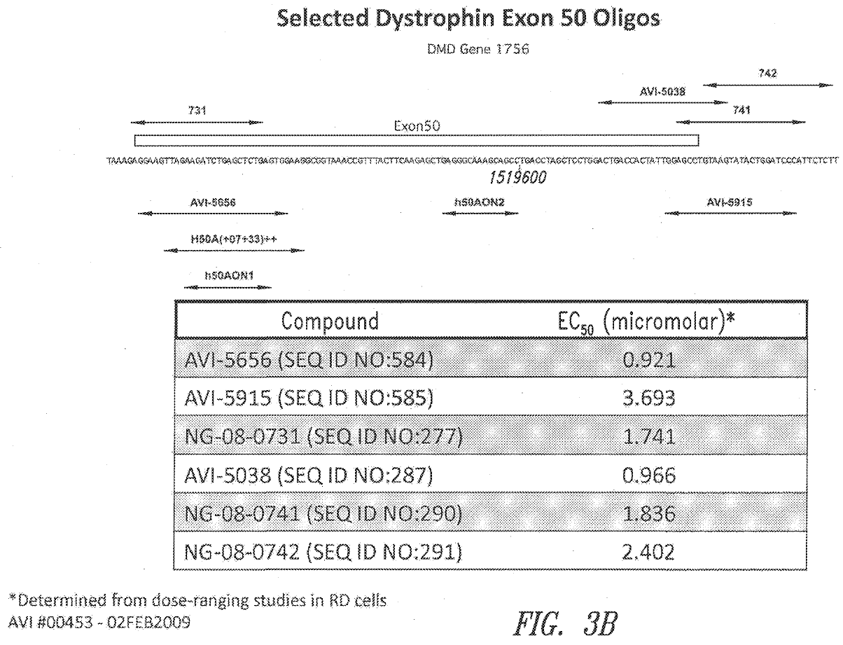

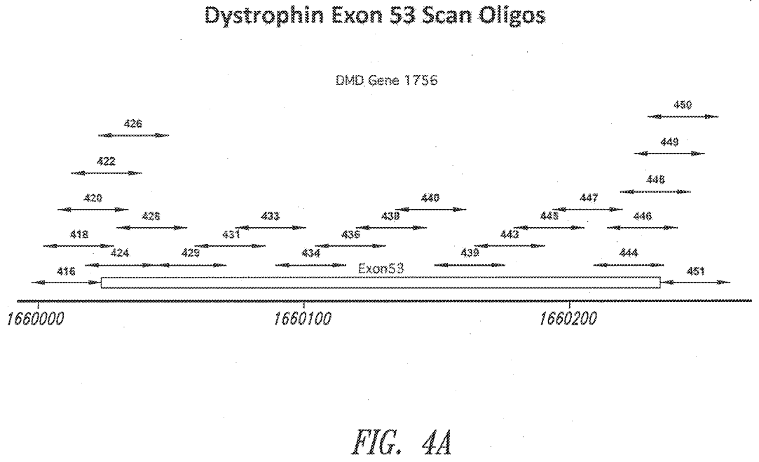

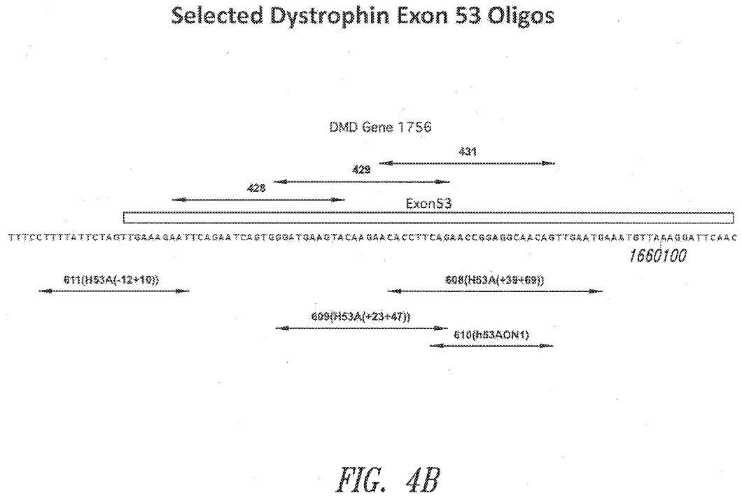

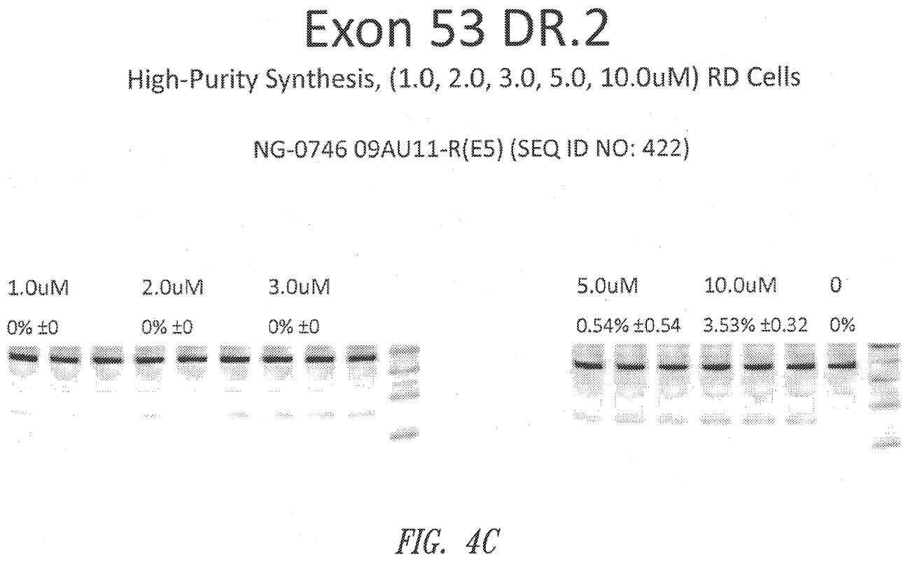

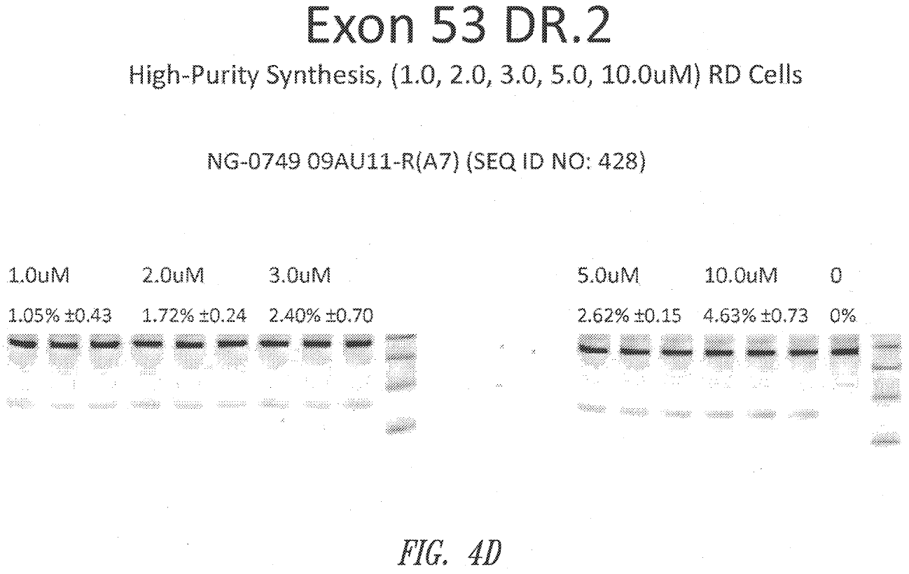

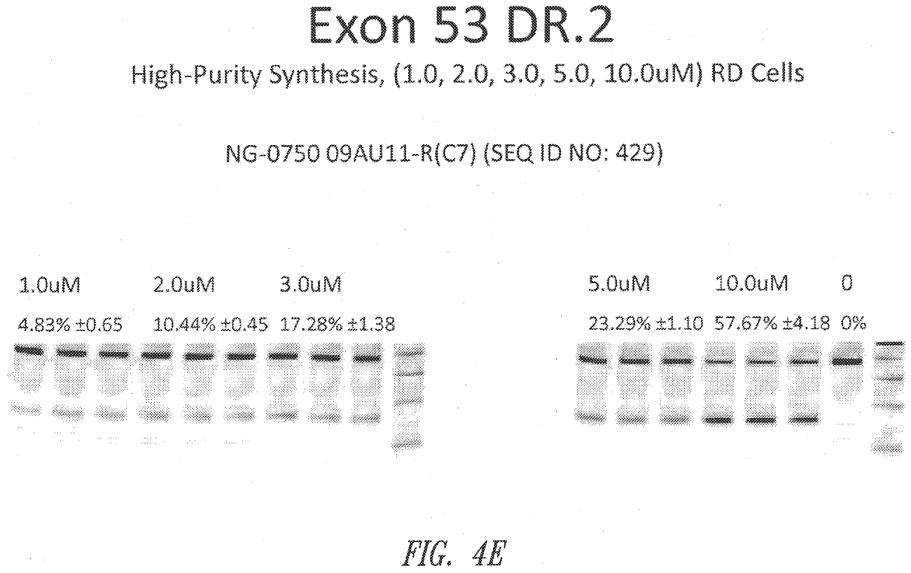

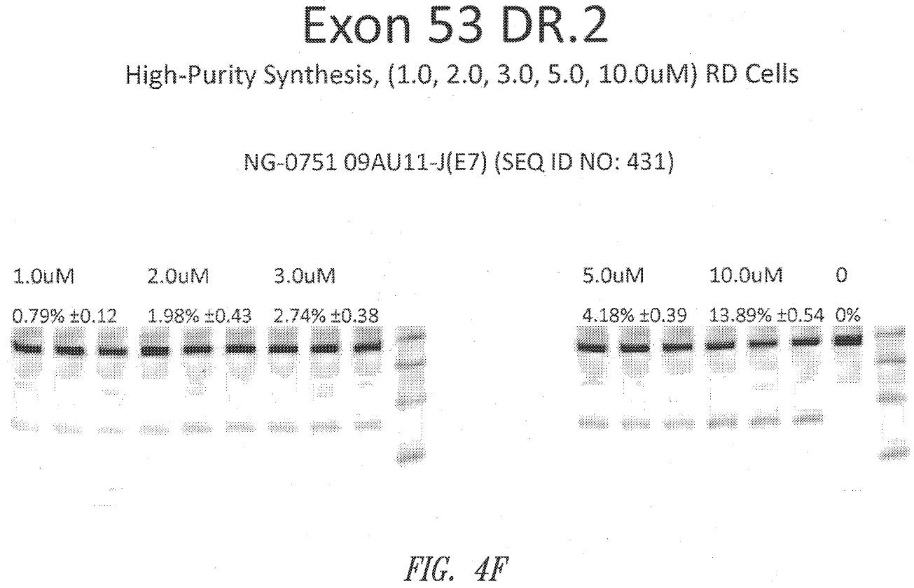

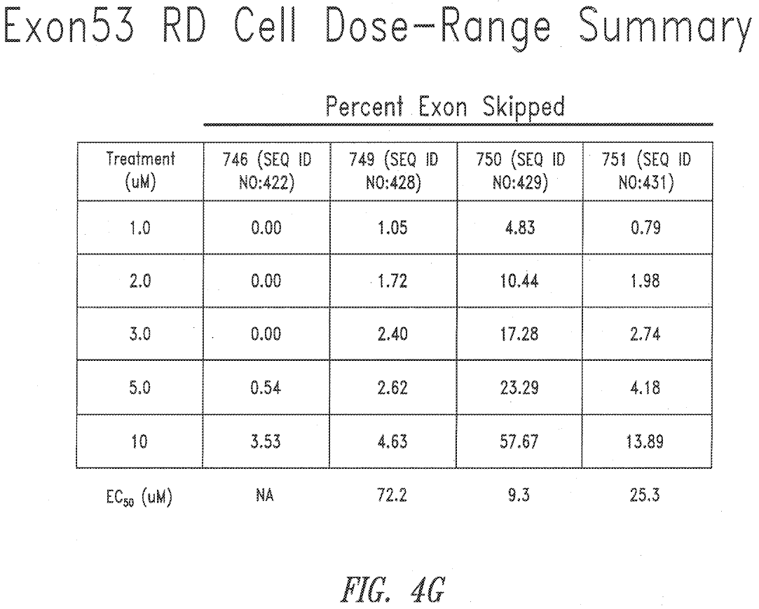

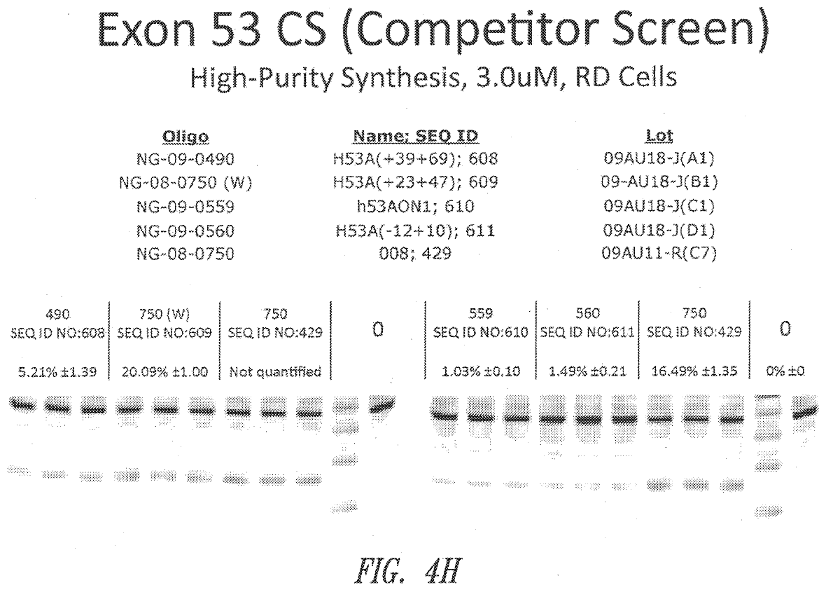

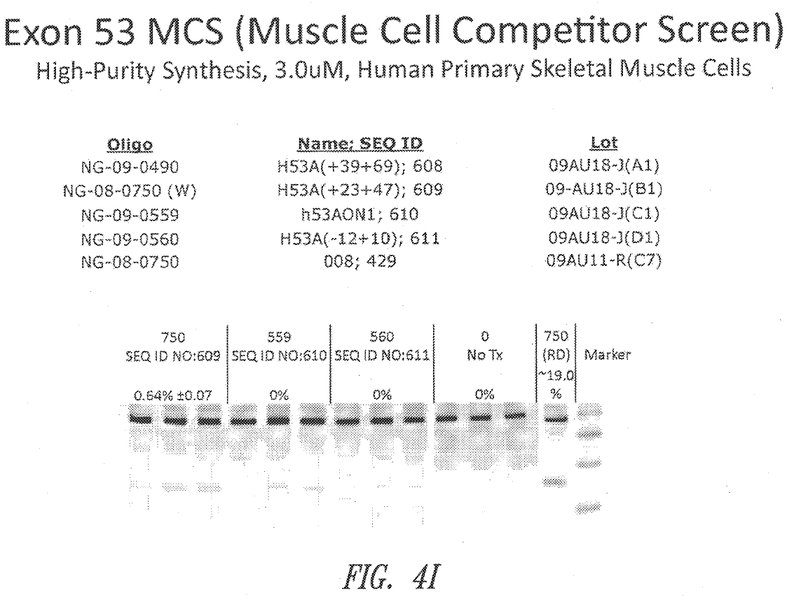

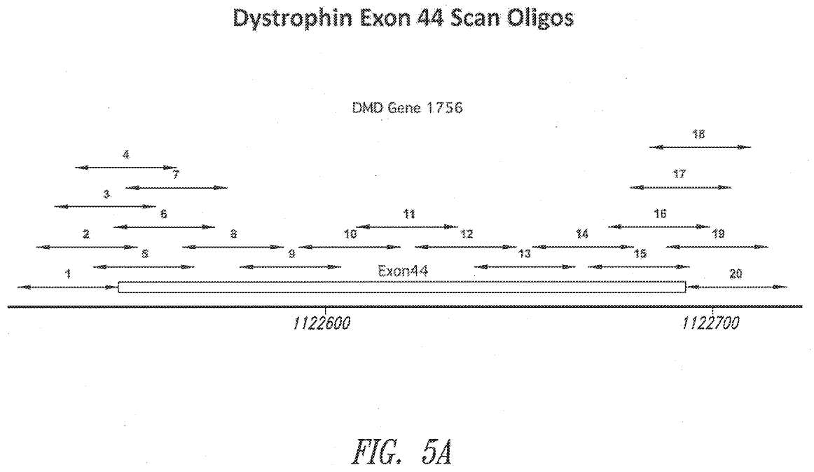

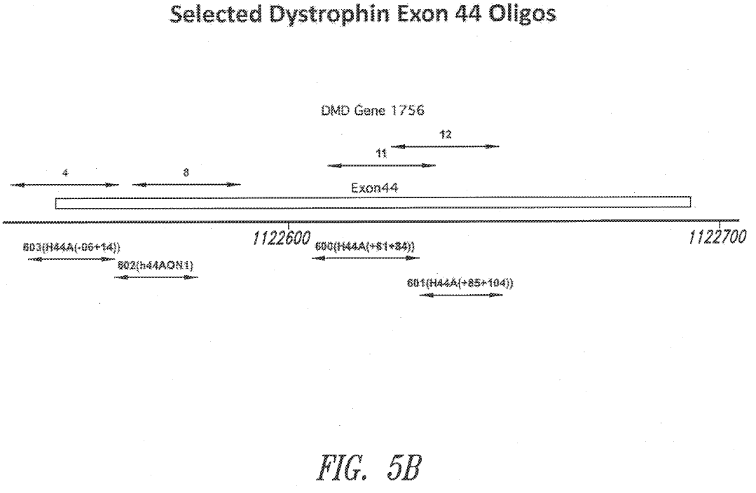

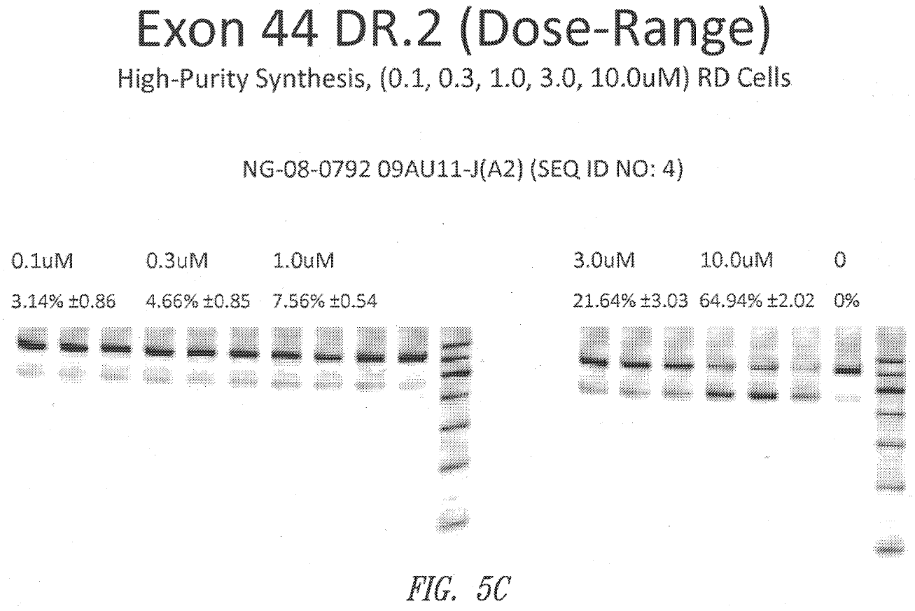

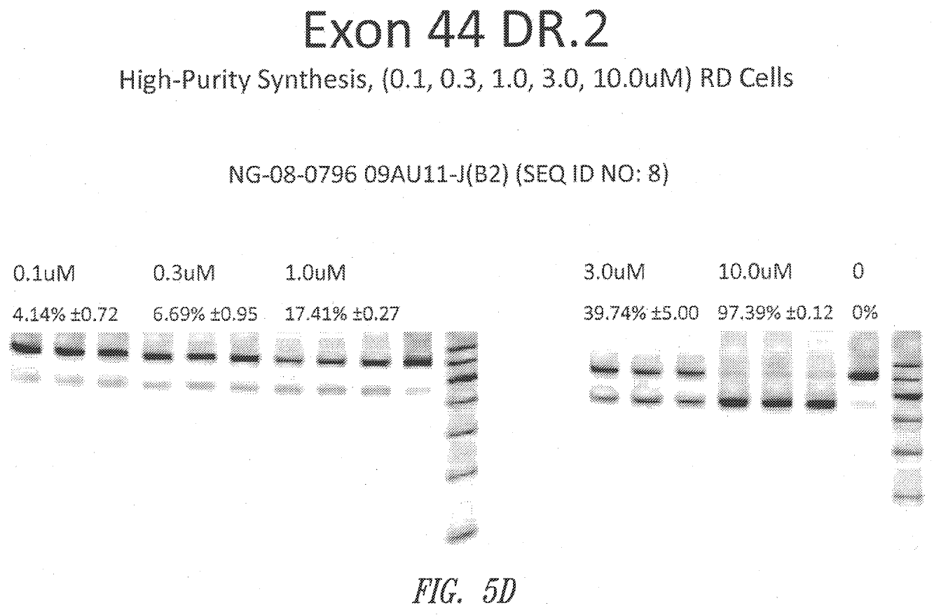

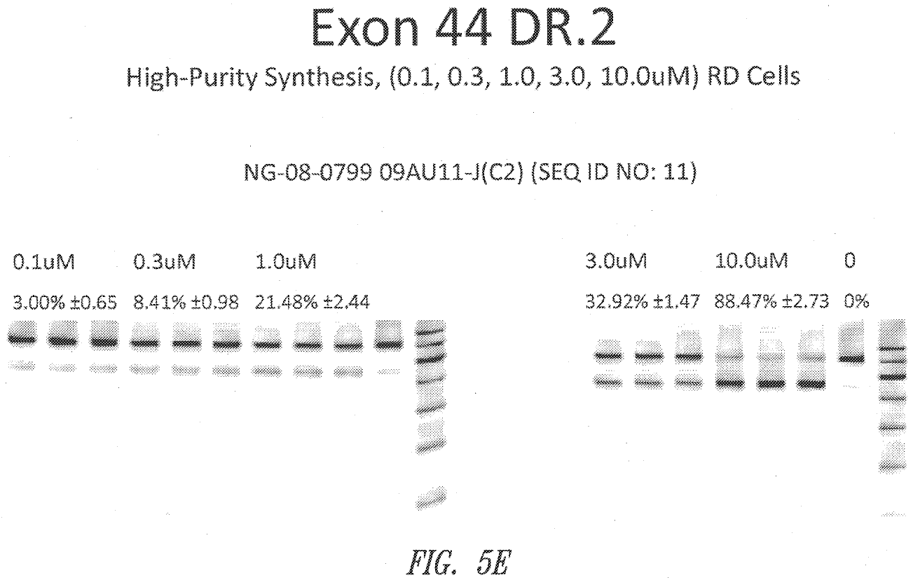

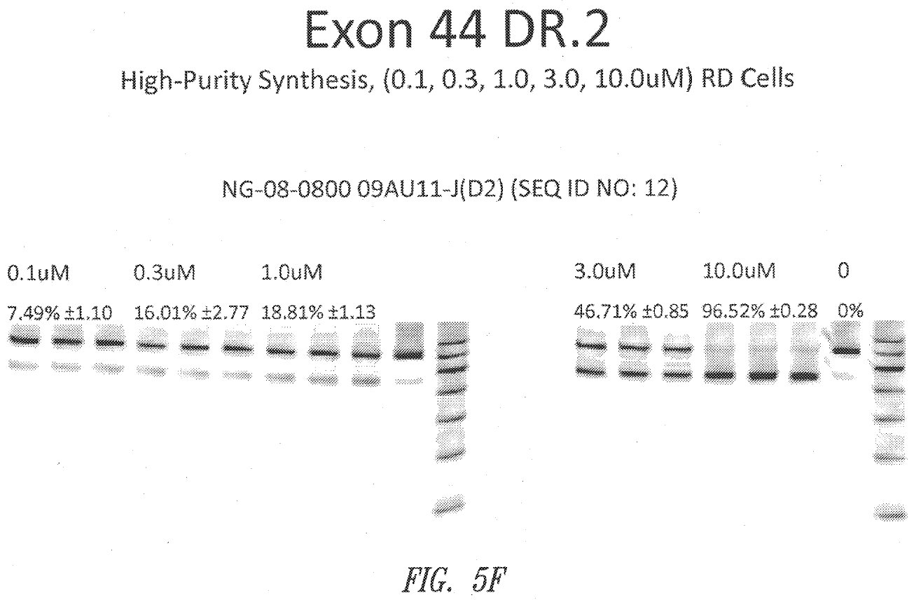

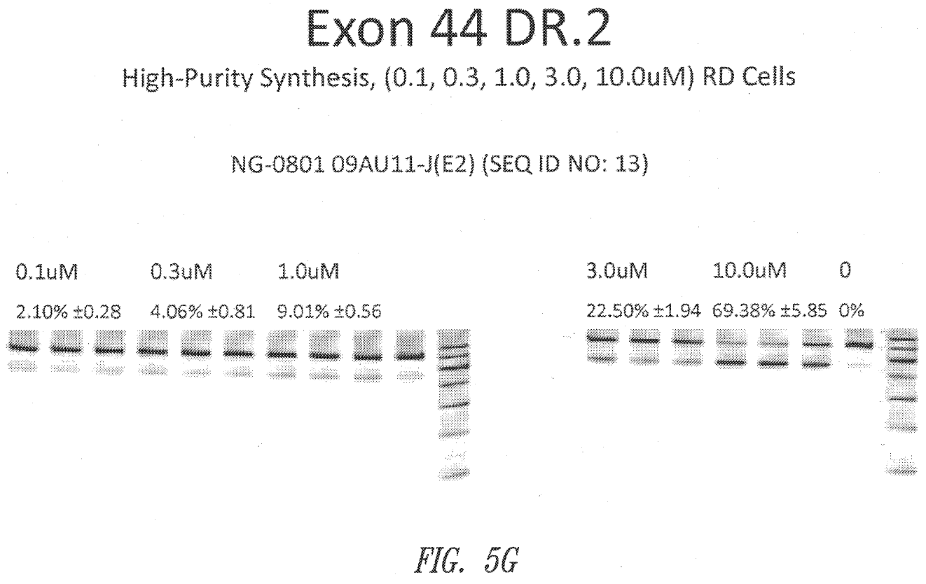

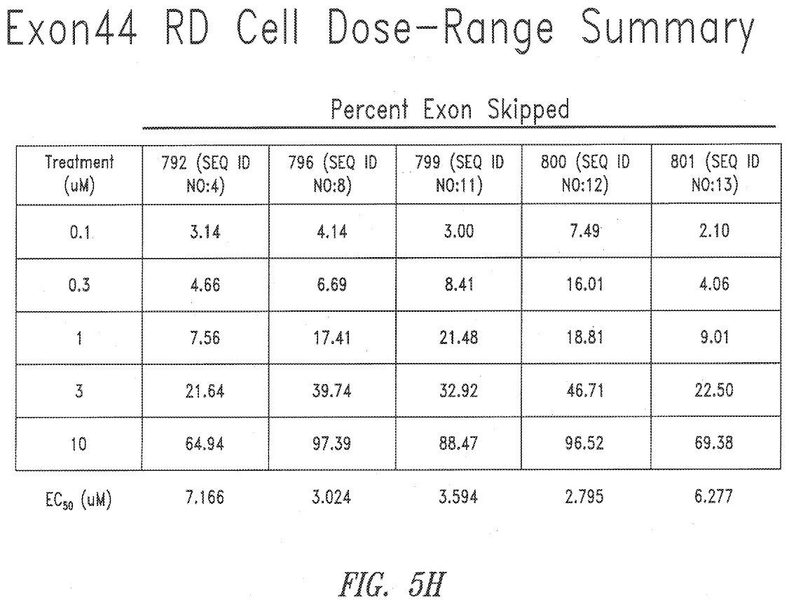

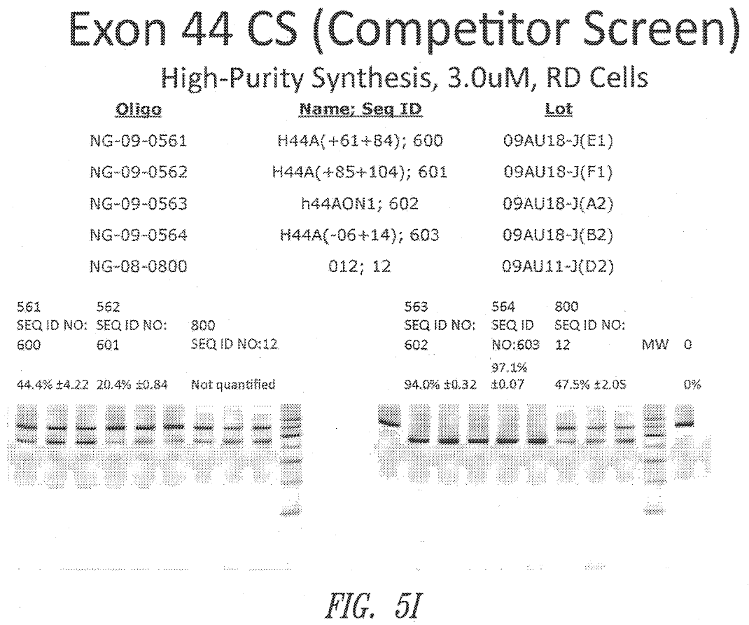

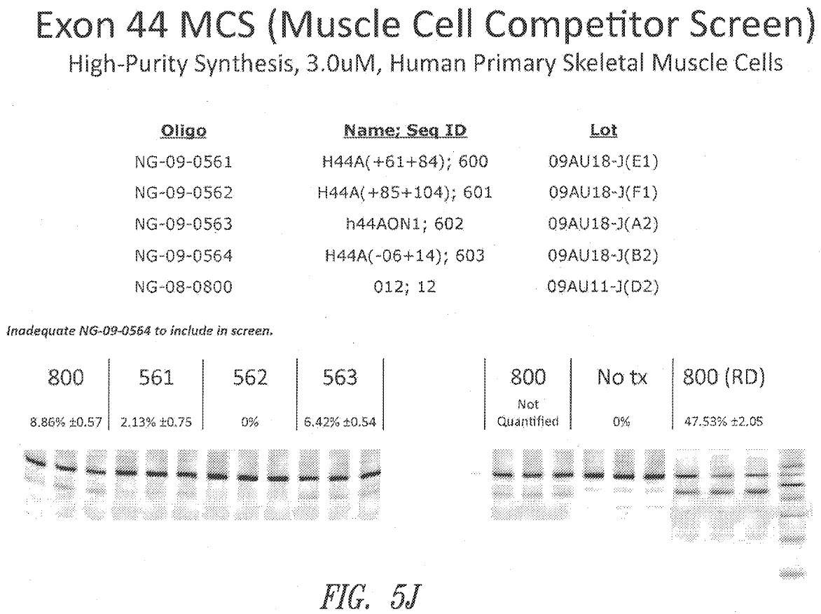

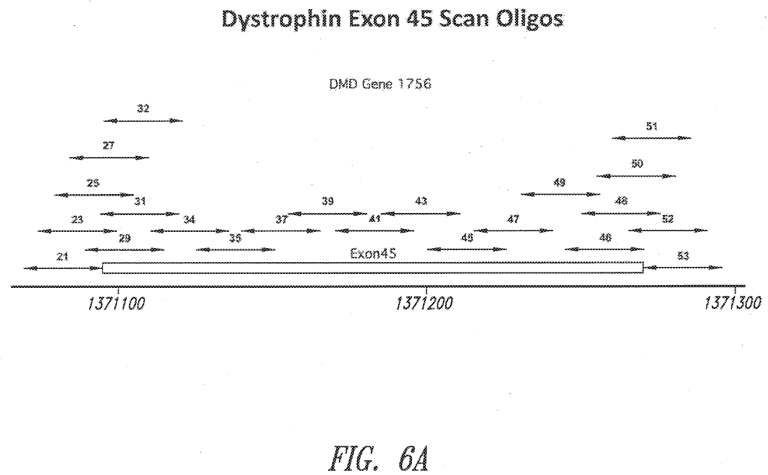

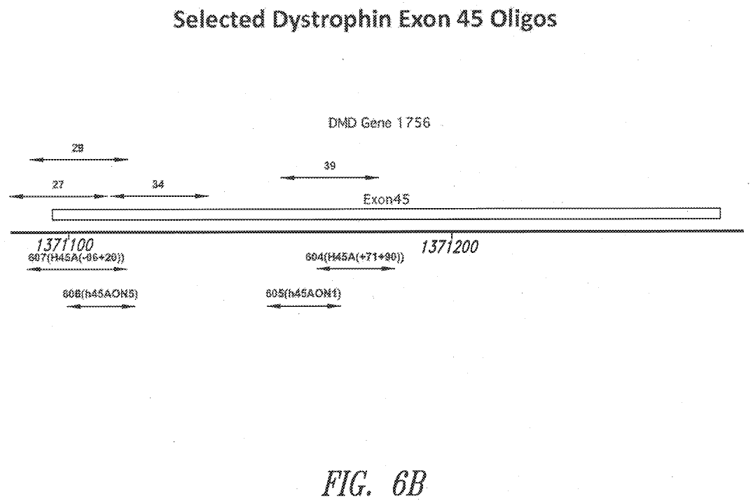

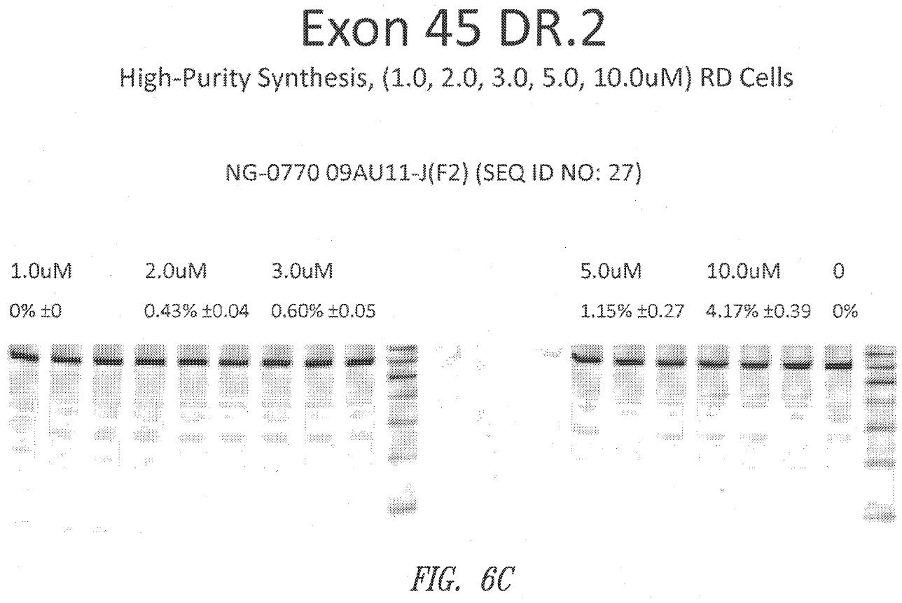

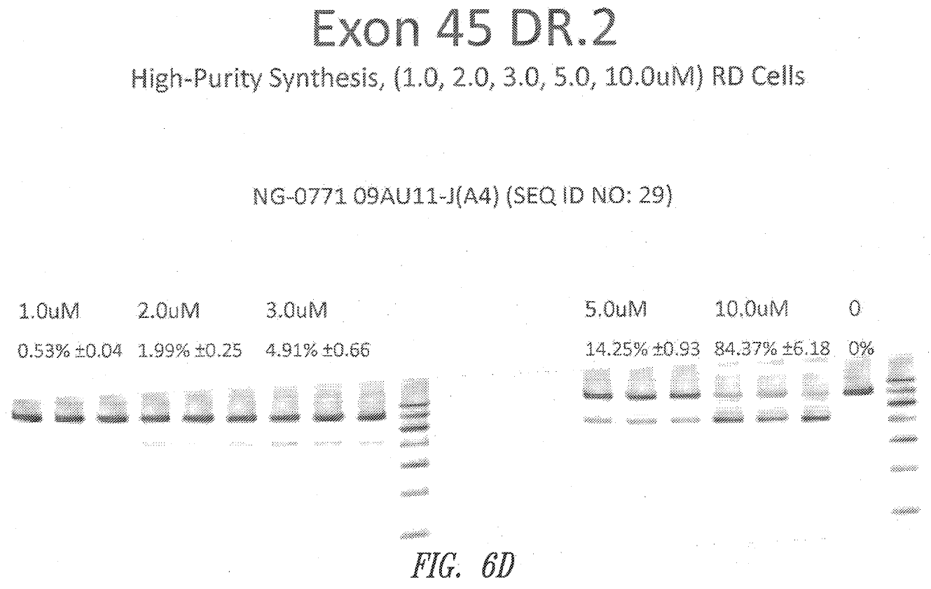

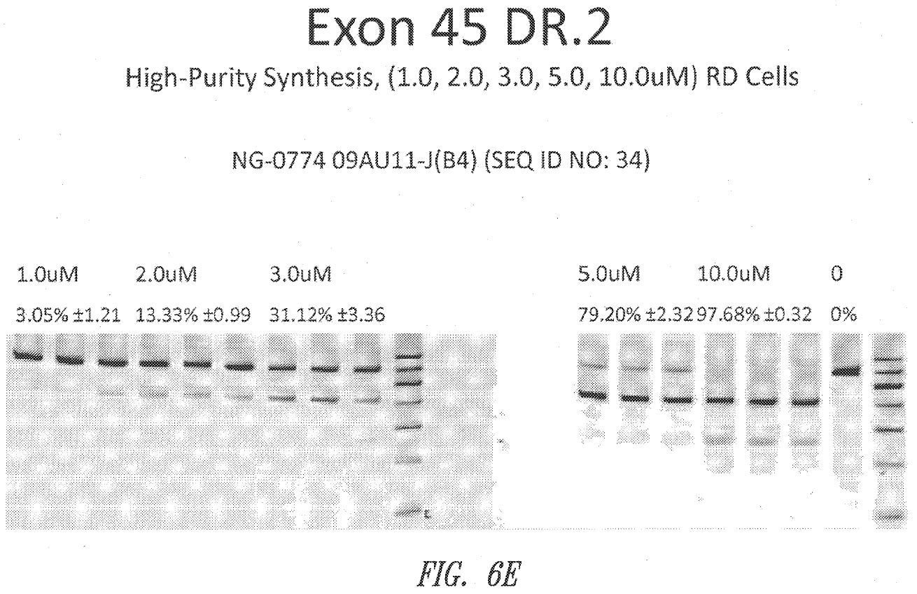

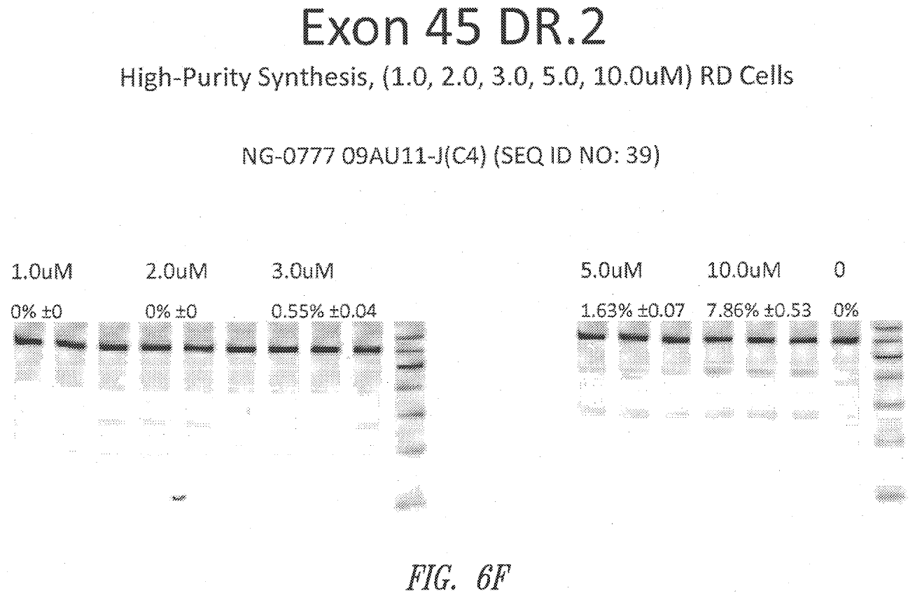

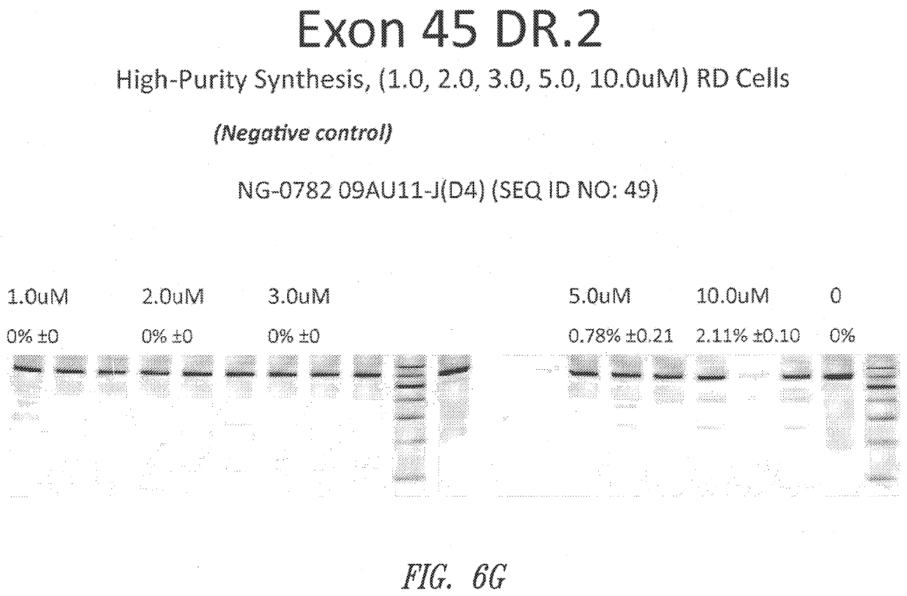

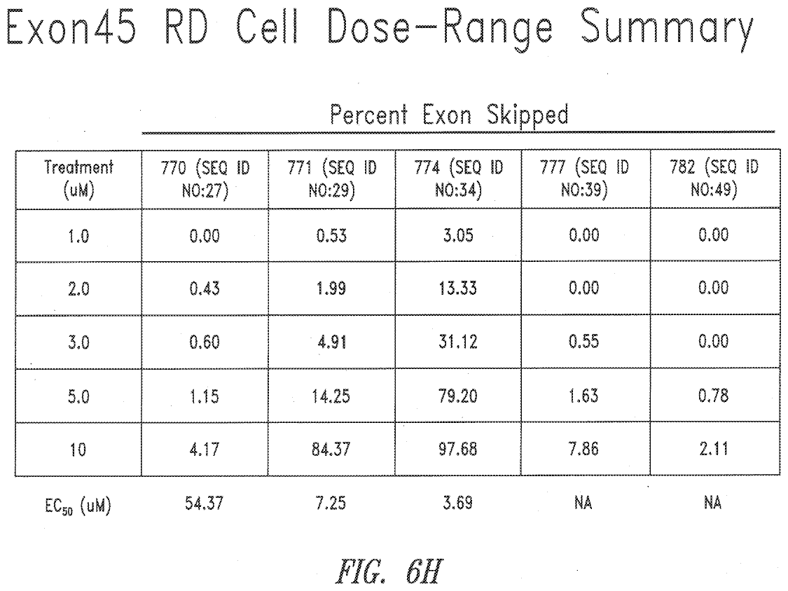

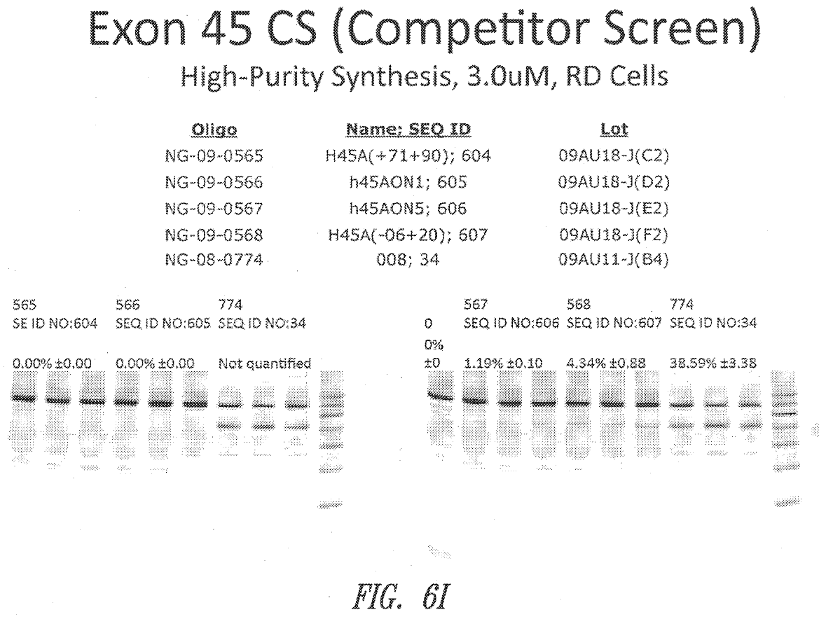

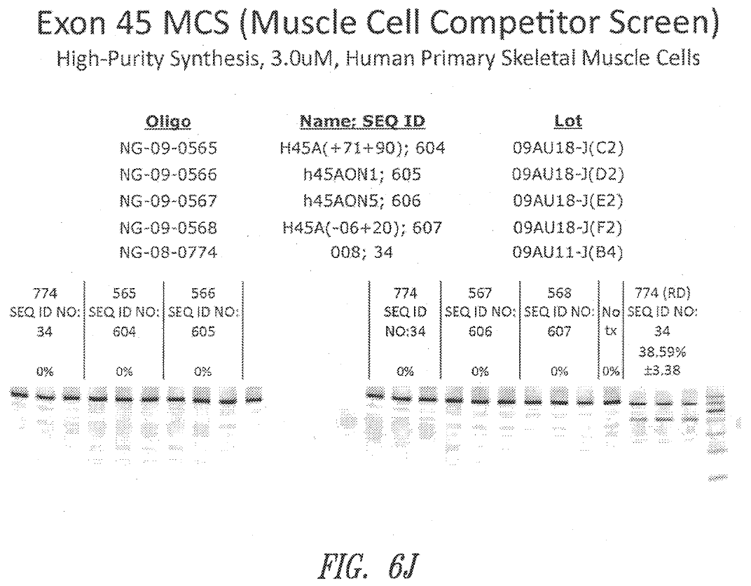

The terms “morpholino oligomer” or “PMO” (phosphoramidate-or phosphorodiamidate morpholino oligomer) refer to an oligonucleotide analog composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide. See, e.g.,, the structure in FIG. 1A, which shows a preferred phosphorodiamidate linkage type. Variations can be made to this linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. See also the discussion of cationic linkages below. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, and PCT Appn. No. PCT/US07/11435 (cationic linkages), all of which are incorporated herein by reference.

The purine or pyrimidine base pairing moiety is typically adenine, cytosine, guanine, uracil, thymine or inosine. Also included are bases such as pyridin-4-one, pyridin-2-one, phenyl, pseudouracil, 2,4,6-trime115thoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl) uridine, 5′-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonyhnethyluridine, 5-methyloxyuridine, 5-methyl-2-thiouridine, 2-methylthio-N6-isopentenyladenosine, β-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine (A), guanine (G), cytosine (C), thymine (T), and uracil (U), as illustrated above; such bases can be used at any position in the antisense molecule. Persons skilled in the art will appreciate that depending on the uses of the oligomers, Ts and Us are interchangeable. For instance, with other antisense chemistries such as 2′-O-methyl antisense oligonucleotides that are more RNA-like, the T bases may be shown as U (see, e.g., Sequencce ID Listing).

An “amino acid subunit” or “amino acid residue” can refer to an α-amino acid residue (e.g., —CO—CHR—NH—) or a β- or other amino acid residue (e.g., —CO—(CH₂)ₙCHR—NH—), where R is a side chain (which may include hydrogen) and n is 1 to 6, preferably 1 to 4.

The term “naturally occurring amino acid” refers to an amino acid present in proteins found in nature, such as the 20 (L)-amino acids utilized during protein biosynthesis as well as others such as 4-hydroxyproline, hydroxylysine, desmosine, isodesmosine, homocysteine, citrulline and ornithine. The term “non-natural amino acids” refers to those amino acids not present in proteins found in nature, examples include beta-alanine (β-Ala; or B), 6-aminohexanoic acid (Ahx) and 6-aminopentanoic acid. Additional examples of “non-natural amino acids” include, without limitation, (D)-amino acids, norleucine, norvaline, p-fluorophenylalanine, ethionine and the like, which are known to a person skilled in the art.

An “effective amount” or “therapeutically effective amount” refers to an amount of therapeutic compound, such as an antisense oligomer, administered to a mammalian subject, either as a single dose or as part of a series of doses, which is effective to produce a desired physiological response or therapeutic effect in the subject. One example of a desired physiological response includes increased expression of a relatively functional or biologically active form of the dystrophin protein, mainly in muscle tissues or cells that contain a defective dystrophin protein or no dystrophin, as compared no antisense oligomer or a control oligomer. Examples of desired therapeutic effects include, without limitation, improvements in the symptoms or pathology of muscular dystrophy, reducing the progression of symptoms or pathology of muscular dystrophy, and slowing the onset of symptoms or pathology of muscular dystrophy, among others. Examples of such symptoms include fatigue, mental retardation, muscle weakness, difficulty with motor skills (e.g., running, hopping, jumping), frequent falls, and difficulty walking. The pathology of muscular dystrophy can be characterized, for example, by muscle fibre damage and membrane leakage. For an antisense oligomer, this effect is typically brought about by altering the splice-processing of a selected target sequence (e.g., dystrophin), such as to induce exon skipping.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 75 exons.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

“Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein. In certain embodiments, the exon being skipped is an aberrant exon from the human dystrophin gene, which may contain a mutation or other alteration in its sequence that otherwise causes aberrant splicing. In certain embodiments, the exon being skipped is any one or more of exons 1-75 of the dystrophin gene, though any one or more of exons 44, 45, 46, 47, 48, 49, 50, 51, 52, 53, 54, and/or 55 of the human dystrophin gene are preferred.

“Dystrophin” is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne's muscular dystrophy (DMD) and Becker's muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain subjects with DMD or BMD, as known in the art, or by the absence of detectable dystrophin.

Table A provides an illustration of the various dystrophin domains, the amino acid residues that encompass these domains, and the exons that encode them.

TABLE A

| | | Residue | |
|---|---|---|---|
| Domain | Sub Domain | Nos | Exons |
| actin binding | | 14-240 | 2-8 |
| domain | | | |
| central rod | | 253-3040 | 8-61 |
| domain | | | |
| | hinge 1 | 253-327 | (8)-9 |
| | repeat 1 | 337-447 | 10-11 |
| | repeat 2 | 448-556 | 12-14 |
| | repeat 3 | 557-667 | 14-16 |
| | hinge 2 | 668-717 | 17 |
| | repeat 4 | 718-828 | (17)-20 |
| | repeat 5 | 829-934 | 20-21 |
| | repeat 6 | 935-1045 | 22-23 |
| | repeat 7 | 1046-1154 | (23)-(26) |
| | repeat 8 | 1155-1263 | 26-27 |
| | repeat 9 | 1264-1367 | 28-(30) |
| | repeat 10 | 1368-1463 | 30-32 |
| | repeat 11 | 1464-1568 | 32-(34) |
| | repeat 12 | 1569-1676 | 34-35 |
| | repeat 13 | 1677-1778 | 36-37 |
| | repeat 14 | 1779-1874 | 38-(40) |
| | repeat 15 | 1875-1973 | 40-41 |
| | interruption | 1974-1991 | 42 |
| | repeat 16 | 1992-2101 | 42-43 |
| | repeat 17 | 2102-2208 | 44-45 |
| | repeat 18 | 2209-2318 | 46-48 |
| | repeat 19 | 2319-2423 | 48-50 |
| | hinge 3 | 2424-2470 | 50-51 |
| | repeat 20 | 2471-2577 | 51-53 |
| | repeat 21 | 2578-2686 | 53-(55) |
| | repeat 22 | 2687-2802 | 55-(57) |
| | repeat 23 | 2803-2931 | 57-59 |
| | repeat 24 | 2932-3040 | 59-(61) |
| | hinge 4 | 3041-3112 | 61-64 |
| Cysteine-rich | | 3080-3360 | 63-69 |
| domain | | | |
| | dystrogiycan | 3080-3408 | 63-70 |
| | binding site | | |
| | WW domain | 3056-3092 | 62-63 |
| | EF-hand 1 | 3130-3157 | 65 |
| | EF-hand 2 | 3178-3206 | 65-66 |
| | ZZ domain | 3307-3354 | 68-69 |
| Carboxy-terminal | | 3361-3685 | 70-79 |
| domain | | | |
| | alpha1-syntrophin | 3444-3494 | 73-74 |
| | binding site | | |
| | ß1-syntrophin | 3495-3535 | 74-75 |
| | binding site | | |
| | (Leu)6-heptad repeat | 3558-3593 | 75 |

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with DMD or BMD. In certain embodiments, a functional dystrophin protein may have about 10%, 20%, 30%, 40%, 50%, 60%, 70%, 80%, 90%, or 100% (including all integers in between) of the in vitro or in vivo biological activity of wild-type dystrophin, as measured according to routine techniques in the art. As one example, dystrophin-related activity in muscle cultures in vitro can be measured according to myotube size, myofibril organization (or disorganization), contractile activity, and spontaneous clustering of acetylcholine receptors (see, e.g., Brown et al., Journal of Cell Science. 112:209-216, 1999). Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84: 165-172, 2003). These and other animal models can be used to measure the functional activity of various dystrophin proteins. Included are truncated forms of dystrophin, such as those forms that are produced by certain of the exon-skipping antisense compounds of the present invention.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

By “isolated” is meant material that is substantially or essentially free from components that normally accompany it in its native state. For example, an “isolated polynucleotide,” as used herein, may refer to a polynucleotide that has been purified or removed from the sequences that flank it in a naturally-occurring state, e.g., a DNA fragment that has been removed from the sequences that are normally adjacent to the fragment.

By “enhance” or “enhancing,” or “increase” or “increasing,” or “stimulate” or “stimulating,” refers generally to the ability of one or antisense compounds or compositions to produce or cause a greater physiological response (i.e., downstream effects) in a cell or a subject, as compared to the response caused by either no antisense compound or a control compound. A measurable physiological response may include increased expression of a functional form of a dystrophin protein, or increased dystrophin-related biological activity in muscle tissue, among other responses apparent from the understanding in the art and the description herein. Increased muscle function can also be measured, including increases or improvements in muscle function by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. The percentage of muscle fibres that express a functional dystrophin can also be measured, including increased dystrophin expression in about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle fibres. For instance, it has been shown that around 40% of muscle function improvement can occur if 25-30% of fibers express dystrophin (see, e.g., DelloRusso et al, Proc Natl Acad Sci USA 99: 12979-12984, 2002). An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1), e.g., 1.5, 1.6, 1.7. 1.8, etc.) the amount produced by no antisense compound (the absence of an agent) or a control compound.

The term “reduce” or “inhibit” may relate generally to the ability of one or more antisense compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of muscular dystrophy, or reductions in the expression of defective forms of dystrophin, such as the altered forms of dystrophin that are expressed in individuals with DMD or BMD. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

“Homology” refers to the percentage number of amino acids that are identical or constitute conservative substitutions. Homology may be determined using sequence comparison programs such as GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence,” “comparison window,” “sequence identity,” “percentage of sequence identity,” and “substantial identity”. A “reference sequence” is at least 8 or 10 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology” John Wiley & Sons Inc, 1994-1998, Chapter 15.

“Treatment” or “treating” of an individual (e.g., a mammal, such as a human) or a cell may include any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

Hence, included are methods of treating muscular dystrophy, such as DMD and BMD, by administering one or more antisense oligomers of the present invention (e.g., SEQ ID NOS: 1 to 569 and 612 to 635, and variants thereof), optionally as part of a pharmaceutical formulation or dosage form, to a subject in need thereof. Also included are methods of inducing exon-skipping in a subject by administering one or more antisense oligomers, in which the exon is one of exons 44, 45, 46, 47, 48, 49, 50, 51, 52, 53, 54, and/or 55 from the dystrophin gene, preferably the human dystrophin gene. A “subject,” as used herein, includes any animal that exhibits a symptom, or is at risk for exhibiting a symptom, which can be treated with an antisense compound of the invention, such as a subject that has or is at risk for having DMD or BMD, or any of the symptoms associated with these conditions (e.g., muscle fibre loss). Suitable subjects (patients) include laboratory animals (such as mouse, rat, rabbit, or guinea pig), farm animals, and domestic animals or pets (such as a cat or dog). Non-human primates and, preferably, human patients, are included.

Also included are vector delivery systems that are capable of expressing the oligomeric, dystrophin-targeting sequences of the present invention, such as vectors that express a polynucleotide sequence comprising any one or more of SEQ ID NOS: 1 to 569 and 612 to 635, or variants thereof, as described herein. By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

A vector or nucleic acid construct system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector or nucleic acid construct is preferably one which is operably functional in a mammalian cell, such as a muscle cell. The vector can also include a selection marker such as an antibiotic or drug resistance gene, or a reporter gene (i.e., green fluorescent protein, luciferase), that can be used for selection or identification of suitable transformants or transfectants. Exemplary delivery systems may include viral vector systems (i.e., viral-mediated transduction) including, but not limited to, retroviral (e.g., lentiviral) vectors, adenoviral vectors, adeno-associated viral vectors, and herpes viral vectors, among others known in the art.

The term “operably linked” as used herein means placing an oligomer-encoding sequence under the regulatory control of a promoter, which then controls the transcription of the oligomer.

A wild-type gene or gene product is that which is most frequently observed in a population and is thus arbitrarily designed the “normal” or “wild-type” form of the gene.

“Alkyl” or “alkylene” both refer to a saturated straight or branched chain hydrocarbon radical containing from 1 to 18 carbons. Examples include without limitation methyl, ethyl, propyl, iso-propyl, butyl, iso-butyl, tert-butyl, n-pentyl and n-hexyl. The term “lower alkyl” refers to an alkyl group, as defined herein, containing between 1 and 8 carbons.

“Alkenyl” refers to an unsaturated straight or branched chain hydrocarbon radical containing from 2 to 18 carbons and comprising at least one carbon to carbon double bond. Examples include without limitation ethenyl, propenyl, iso-propenyl, butenyl, iso-butenyl, tert-butenyl, n-pentenyl and n-hexenyl. The term “lower alkenyl” refers to an alkenyl group, as defined herein, containing between 2 and 8 carbons.

“Alkynyl” refers to an unsaturated straight or branched chain hydrocarbon radical containing from 2 to 18 carbons comprising at least one carbon to carbon triple bond. Examples include without limitation ethynyl, propynyl, iso-propynyl, butynyl, iso-butynyl, tert-butynyl, pentynyl and hexynyl. The term “lower alkynyl” refers to an alkynyl group, as defined herein, containing between 2 and 8 carbons.

“Cycloalkyl” refers to a mono-or poly-cyclic alkyl radical. Examples include without limitation cyclobutyl, cycopentyl, cyclohexyl, cycloheptyl and cyclooctyl.

“Aryl” refers to a cyclic aromatic hydrocarbon moiety containing from 5 to 18 carbons having one or more closed ring(s). Examples include without limitation phenyl, benzyl, naphthyl, anthracenyl, phenanthracenyl and biphenyl.

“Aralkyl” refers to a radical of the formula RaRb where Ra is an alkylene chain as defined above and Rb is one or more aryl radicals as defined above, for example, benzyl, diphenylmethyl and the like.

“Thioalkoxy” refers to a radical of the formula-SRc where Rc is an alkyl radical as defined herein. The term “lower thioalkoxy” refers to an alkoxy group, as defined herein, containing between 1 and 8 carbons.

“Alkoxy” refers to a radical of the formula-ORda where Rd is an alkyl radical as defined herein. The term “lower alkoxy” refers to an alkoxy group, as defined herein, containing between 1 and 8 carbons. Examples of alkoxy groups include, without limitation, methoxy and ethoxy.

“Alkoxyalkyl” refers to an alkyl group substituted with an alkoxy group.

“Carbonyl” refers to the —C(═O)— radical.

“Guanidynyl” refers to the H₂N(C═NH₂)—NH— radical.

“Amidinyl” refers to the H₂N(C═NH₂)CH— radical.

“Amino” refers to the —NH₂ radical.

“Alkylamino” refers to a radical of the formula —NHRd or —NRdRd where each Rd is, independently, an alkyl radical as defined herein. The term “lower alkylamino” refers to an alkylamino group, as defined herein, containing between 1 and 8 carbons.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined below. Thus, in addition to the heteroaryls listed below, heterocycles also include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydrothiophenyl, tetrahydrothiopyranyl, tetrahydropyrimidinyl, tetrahydrothiopyranyl, and the like.

“Heteroaryl” means an aromatic heterocycle ring of 5- to 10 members and having at least one heteroatom selected from nitrogen, oxygen and sulfur, and containing at least 1 carbon atom, including both mono-and bicyclic ring systems. Representative heteroaryls are pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

The terms “optionally substituted alkyl”, “optionally substituted alkenyl”, “optionally substituted alkoxy”, “optionally substituted thioalkoxy”, “optionally substituted alkyl amino”, “optionally substituted lower alkyl”, “optionally substituted lower alkenyl”, “optionally substituted lower alkoxy”, “optionally substituted lower thioalkoxy”, “optionally substituted lower alkyl amino” and “optionally substituted heterocyclyl” mean that, when substituted, at least one hydrogen atom is replaced with a substituent. In the case of an oxo substituent (=O) two hydrogen atoms are replaced. In this regard, substituents include: deuterium, optionally substituted alkyl, optionally substituted alkenyl, optionally substituted alkynyl, optionally substituted aryl, optionally substituted heterocycle, optionally substituted cycloalkyl, oxo, halogen, —CN, —ORx, NRxRy, NRxC(═O)Ry, NRxSO2Ry, —NRxC(═O)NRxRy, C(═O)Rx, C(═O)ORx, C(═O)NRxRy, —SOmRx and —SOmN RxRy, wherein m is 0, 1 or 2, Rx and Ry are the same or different and independently hydrogen, optionally substituted alkyl, optionally substituted alkenyl, optionally substituted alkynyl, optionally substituted aryl, optionally substituted heterocycle or optionally substituted cycloalkyl and each of said optionally substituted alkyl, optionally substituted alkenyl, optionally substituted alkynyl, optionally substituted aryl, optionally substituted heterocycle and optionally substituted cycloalkyl substituents may be further substituted with one or more of oxo, halogen, —CN, —ORx, NRxRy, NRxC(═O)Ry, NRxSO 2—Ry, —NRxC(═O)NRxRy, C(═O)Rx, C(═O)ORx, C(═O)NRxRy, —SOmRx and —SOmN RxRy.

### Constructing Antisense Oligonucleotides

Examples of morpholino oligonucleotides having phosphorus-containing backbone linkages are illustrated in FIGS. 1A-1C. Especially preferred is a phosphorodiamidate-linked morpholino oligonucleotide such as shown in FIG. 1C, which is modified, in accordance with one aspect of the present invention, to contain positively charged groups at preferably 10%-50% of its backbone linkages. Morpholino oligonucleotides with uncharged backbone linkages and their preparation, including antisense oligonucleotides, are detailed, for example, in (Summerton and Weller 1997) and in co-owned U.S. Pat. Nos. 5,698,685, 5,217,866, 5,142,047, 5,034,506, 5,166,315, 5,185, 444, 5,521,063, and 5,506,337, all of which are expressly incorporated by reference herein.

Important properties of the morpholino-based subunits include: 1) the ability to be linked in a oligomeric form by stable, uncharged or positively charged backbone linkages; 2) the ability to support a nucleotide base (e.g. adenine, cytosine, guanine, thymidine, uracil and inosine) such that the polymer formed can hybridize with a complementary-base target nucleic acid, including target RNA, Tm values above about 45° C. in relatively short oligonucleotides (e.g., 10-15 bases); 3) the ability of the oligonucleotide to be actively or passively transported into mammalian cells; and 4) the ability of the antisense oligonucleotide: RNA heteroduplex to resist RNAse and RNaseH degradation, respectively.

Exemplary backbone structures for antisense oligonucleotides of the claimed subject matter include the morpholino subunit types shown in FIGS. 1D-G, each linked by an uncharged or positively charged, phosphorus-containing subunit linkage. FIG. 1D shows a phosphorus-containing linkage which forms the five atom repeating-unit backbone, wherein the morpholino rings are linked by a 1-atom phosphoamide linkage. FIG. 1E shows a linkage which produces a 6-atom repeating-unit backbone. In this structure, the atom Y linking the 5′ morpholino carbon to the phosphorus group may be sulfur, nitrogen, carbon or, preferably, oxygen. The X moiety pendant from the phosphorus may be fluorine, an alkyl or substituted alkyl, an alkoxy or substituted alkoxy, a thioalkoxy or substituted thioalkoxy, or unsubstituted, monosubstituted, or disubstituted nitrogen, including cyclic structures, such as morpholines or piperidines. Alkyl, alkoxy and thioalkoxy preferably include 1-6 carbon atoms. The Z moieties are sulfur or oxygen, and are preferably oxygen.

The linkages shown in FIGS. 1F and 1G are designed for 7-atom unit-length backbones. In structure 1F, the X moiety is as in Structure 1E, and the Y moiety may be methylene, sulfur, or, preferably, oxygen. In Structure 1G, the X and Y moieties are as in Structure 1E. Particularly preferred morpholino oligonucleotides include those composed of morpholino subunit structures of the form shown in FIG. 1E, where X═NH₂, N(CH₃)₂, optionally substituted 1-piperazinyl, or other charged group, Y═O, and Z═O.

As noted above, the uncharged or substantially uncharged oligonucleotide may be modified, in accordance with an aspect of the invention, to include charged linkages, e.g. up to about 1 per every 2-5 uncharged linkages, such as about 4-5 per every 10 uncharged linkages. Optimal improvement in antisense activity may be seen when about 25% of the backbone linkages are cationic, including about 20% to about 30%. Also included are oligomers in which about 35%, 40%, 45%, 50%, 55%, 60% (including all integers in between), or more of the backbone linkages are cationic. Enhancement is also seen with a small number, e.g., 5% or 10-20%, of cationic linkages.

A substantially uncharged, phosphorus containing backbone in an oligonucleotide analog is typically one in which a majority of the subunit linkages, e.g., between 50%-100%, typically at least 60% to 100% or 75% or 80% of its linkages, are uncharged at physiological pH and contain a single phosphorous atom.

Additional experiments conducted in support of the present invention indicate that the enhancement seen with added cationic backbone charges may, in some cases, be further enhanced by distributing the bulk of the charges close to the “center-region” backbone linkages of the antisense oligonucleotide, e.g., in a 20 mer oligonucleotide with 8 cationic backbone linkages, having at least 70% of these charged linkages localized in the 10 centermost linkages.

The antisense compounds can be prepared by stepwise solid-phase synthesis, employing methods detailed in the references cited above, and below with respect to the synthesis of oligonucleotides having a mixture of uncharged and cationic backbone linkages. In some cases, it may be desirable to add additional chemical moieties to the antisense compound, e.g. to enhance pharmacokinetics or to facilitate capture or detection of the compound. Such a moiety may be covalently attached, typically to a terminus of the oligomer, according to standard synthetic methods. For example, addition of a polyethyleneglycol moiety or other hydrophilic polymer, e.g., one having 10-100 monomeric subunits, may be useful in enhancing solubility. One or more charged groups, e.g., anionic charged groups such as an organic acid, may enhance cell uptake. A reporter moiety, such as fluorescein or a radiolabeled group, may be attached for purposes of detection. Alternatively, the reporter label attached to the oligomer may be a ligand, such as an antigen or biotin, capable of binding a labeled antibody or streptavidin. In selecting a moiety for attachment or modification of an antisense compound, it is generally of course desirable to select chemical compounds of groups that are biocompatible and likely to be tolerated by a subject without undesirable side effects.

As noted above, the antisense compound can be constructed to contain a selected number of cationic linkages interspersed with uncharged linkages of the type described above. The intersubunit linkages, both uncharged and cationic, preferably are phosphorus-containing linkages, having the structure (II):

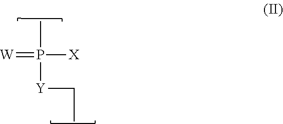

wherein:

W is —S— or —O—, and is preferably —O—,

X═—NR¹R² or —OR⁶,

Y═—O— or —NR⁷, and

each said linkage in the oligomer is selected from:

(a) an uncharged linkage (a), wherein each of R¹, R², R⁶ and R⁷ is independently selected from hydrogen and lower alkyl;

(b1) a cationic linkage (b1), wherein X═—NR¹R² and Y═—O—, and —NR¹R² represents an optionally substituted piperazinyl moiety, such that R¹R²═—CHRCHRN(R³)(R⁴)CHRCHR—, wherein:

each R is independently H or —CH₃,

R⁴ is H, —CH₃, or an electron pair, and

R³ is selected from H, optionally substituted lower alkyl, —C(═NH)NH₂, —Z—L—NHC(═NH)NH₂, and [—C(═O)CHR′NH]ₘH, where: Z is —C(═O)— or a direct bond, L is an optional linker up to 18 atoms in length, preferably up to 12 atoms, and more preferably up to 8 atoms in length, having bonds selected from optionally substituted alkyl, optionally substituted alkoxy, and optionally substituted alkylamino, R′ is a side chain of a naturally occurring amino acid or a one- or two-carbon homolog thereof, and m is 1 to 6, preferably 1 to 4;

(b2) a cationic linkage (b2), wherein X═—NR¹R² and Y═—O—, R¹═H or —CH₃, and R²═LNR³R⁴R⁵, wherein L, R³, and R⁴ are as defined above, and R⁵ is H, optionally substituted lower alkyl, or optionally substituted lower (alkoxy)alkyl; and

(b3) a cationic linkage (b3), wherein Y═—NR⁷ and X═—OR⁶, and R⁷═—LNR³R⁴R⁵, wherein L, R³, R⁴ and R⁵ are as defined above, and R⁶ is H or optionally substituted lower alkyl; and

at least one said linkage is selected from cationic linkages (b1), (b2), and (b3).

Preferably, the oligomer includes at least two consecutive linkages of type (a) (i.e. uncharged linkages). In further embodiments, at least 5% of the linkages in the oligomer are cationic linkages (i.e. type (b1), (b2), or (b3)); for example, 10% to 60%, and preferably 20-50% linkages may be cationic linkages.

In one embodiment, at least one linkage is of type (b1), where, preferably, each R is H, R⁴ is H, —CH₃, or an electron pair, and R³ is selected from H, optionally substituted lower alkyl, —C(═NH)NH₂, and —C(═O)—L—NHC(═NH)NH₂ The latter two embodiments of R³ provide a guanidino moiety, either attached directly to the piperazine ring, or pendant to a linker group L, respectively. For ease of synthesis, the variable Z in R³ is preferably —C(═O)—, as shown.

The linker group L, as noted above, contains bonds in its backbone selected from optionally substituted alkyl, optionally substituted alkoxy, and optionally substituted alkylamino, wherein the terminal atoms in L (e.g., those adjacent to carbonyl or nitrogen) are carbon atoms. Although branched linkages are possible, the linker is preferably unbranched. In one embodiment, the linker is a linear alkyl linker. Such a linker may have the structure —(CH₂)ₙ—, where n is 1-12, preferably 2-8, and more preferably 2-6.

The morpholino subunits have the following structure (III):

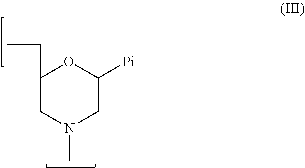

wherein Pi is a base-pairing moiety, and the linkages depicted above connect the nitrogen atom of (III) to the 5′ carbon of an adjacent subunit. The base-pairing moieties Pi may be the same or different, and are generally designed to provide a sequence which binds to a target nucleic acid.

The use of embodiments of linkage types (b1), (b2) and (b3) above to link morpholino subunits (III) may be illustrated graphically as follows:

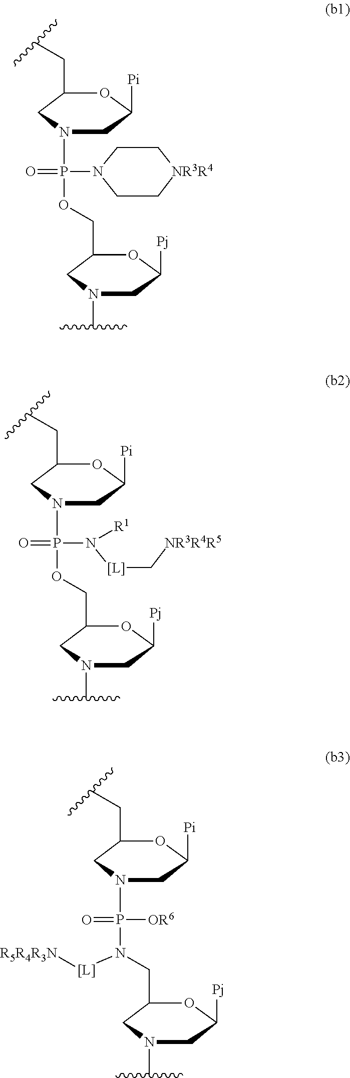

Preferably, all cationic linkages in the oligomer are of the same type; i.e. all of type (b1), all of type (b2), or all of type (b3).

In further embodiments, the cationic linkages are selected from linkages (b1′) and (b1″) as shown below, where (b1′) is referred to herein as a “Pip” linkage and (b1″) is referred to herein as a “GuX” linkage:

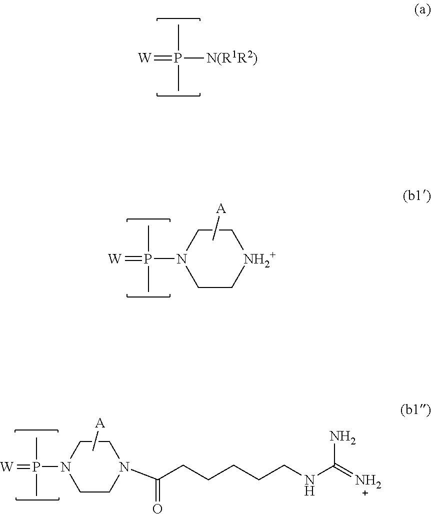

In the structures above, W is S or O, and is preferably O; each of R¹ and R² is independently selected from hydrogen and optionally substituted lower alkyl, and is preferably methyl; and A represents hydrogen or a non-interfering substituent (i.e. a substituent that does not adversely affect the ability of an oligomer to bind to its intended target) on one or more carbon atoms in (b1′) and (b1″). Preferably, the ring carbons in the piperazine ring are unsubstituted; however, the ring carbons of the piperazine ring may include non-interfering substituents, such as methyl or fluorine. Preferably, at most one or two carbon atoms is so substituted.

In further embodiments, at least 10% of the linkages are of type (b1′) or (b1″); for example, 10%-60% and preferably 20% to 50%, of the linkages may be of type (b1′) or (b1″).

In other embodiments, the oligomer contains no linkages of the type (b1′) above. Alternatively, the oligomer contains no linkages of type (b1) where each R is H, R³ is H or —CH₃, and R⁴ is H, —CH₃, or an electron pair.

The morpholino subunits may also be linked by non-phosphorus-based intersubunit linkages, as described further below, where at least one linkage is modified with a pendant cationic group as described above.

Other oligonucleotide analog linkages which are uncharged in their unmodified state but which could also bear a pendant amine substituent could be used. For example, a 5′nitrogen atom on a morpholino ring could be employed in a sulfamide linkage or a urea linkage (where phosphorus is replaced with carbon or sulfur, respectively) and modified in a manner analogous to the 5′-nitrogen atom in structure (b3) above.

Oligomers having any number of cationic linkages are provided, including fully cationic-linked oligomers. Preferably, however, the oligomers are partially charged, having, for example, 10%-80%. In preferred embodiments, about 10% to 60%, and preferably 20% to 50% of the linkages are cationic.

In one embodiment, the cationic linkages are interspersed along the backbone. The partially charged oligomers preferably contain at least two consecutive uncharged linkages; that is, the oligomer preferably does not have a strictly alternating pattern along its entire length.

Also considered are oligomers having blocks of cationic linkages and blocks of uncharged linkages; for example, a central block of uncharged linkages may be flanked by blocks of cationic linkages, or vice versa. In one embodiment, the oligomer has approximately equal-length 5′, 3′ and center regions, and the percentage of cationic linkages in the center region is greater than about 50%, preferably greater than about 70%.

Oligomers for use in antisense applications generally range in length from about 10 to about 40 subunits, more preferably about 10 to 30 subunits, and typically 15-25 bases, including those having 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases. In certain embodiments, an oligomer of the invention having 19-20 subunits, a useful length for an antisense compound, may ideally have two to ten, e.g., four to eight, cationic linkages, and the remainder uncharged linkages. An oligomer having 14-15 subunits may ideally have two to seven, e.g., 3 to 5, cationic linkages, and the remainder uncharged linkages.

Each morpholino ring structure supports a base pairing moiety, to form a sequence of base pairing moieties which is typically designed to hybridize to a selected antisense target in a cell or in a subject being treated. The base pairing moiety may be a purine or pyrimidine found in native DNA or RNA (e.g., A, G, C, T or U) or an analog, such as hypoxanthine (the base component of the nucleoside inosine) or 5-methyl cytosine.

### Peptide Transporters

The antisense compounds of the invention may include an oligonucleotide moiety conjugated to an arginine-rich peptide transport moiety effective to enhance transport of the compound into cells. The transport moiety is preferably attached to a terminus of the oligomer, as shown, for example, in FIGS. 1B and 1C. The peptide transport moiety preferably comprises 6 to 16 subunits selected from X′ subunits, Y subunits, and Z′ subunits,

wherein:

(a) each X′ subunit independently represents lysine, arginine or an arginine analog, said analog being a cationic α-amino acid comprising a side chain of the structure R¹N═C(NH₂)R², where R¹ is H or R; R² is R, —NH₂, —NHR, or —NR₂, where R is optionally substituted lower alkyl or optionally substituted lower alkenyl; R¹ and R² may join together to form a ring; and the side chain is linked to said amino acid via R¹ or R²;

(b) each Y′ subunit independently represents a neutral amino acid —C(═O)—(CHR)ₙ—NH—, where n is 2 to 7 and each R is independently H or methyl; and

(c) each Z′ subunit independently represents an α-amino acid having a neutral aralkyl side chain;

wherein the peptide comprises a sequence represented by at least one of (X′Y′X′)ₚ, (X′Y′)_{m,} and/or (X′Z′Z′)ₚ, where p is 2 to 5 and m is 2 to 8. Certain embodiments include various combinations selected independently from (X′Y′X′)ₚ, (X′Y″)ₘ, and/or (X′Z′Z′)ₚ, including, for example, peptides having the sequence (X′Y′X′)(X′Z′Z′)(X′Y′X′)(X′Z′Z′) (SEQ ID NO:637).

In selected embodiments, for each X′, the side chain moiety is guanidyl, as in the amino acid subunit arginine (Arg). In certain embodiments, each Y′ is independently —C(═O)—(CH₂)_{n—}CHR—NH—, where n is 2 to 7 and R is H. For example, when n is 5 and R is H, Y′ is a 6-aminohexanoic acid subunit, abbreviated herein as Ahx; when n is 2 and R is H, Y′ is a β-alanine subunit, abbreviated herein as B. Certain embodiments relate to carrier peptides having a combination of different neutral amino acids, including, for example, peptides comprising the sequence —RahxRRBRRAhxRRBRAhxB— (SEQ ID NO:578), which contains both β-alanine and 6-aminohexanoic acid.

Preferred peptides of this type include those comprising arginine dimers alternating with single Y′ subunits, where Y′ is preferably Ahx or B or both. Examples include peptides having the formula (RY′R)ₚ and/or the formula (RRY′)ₚ, where p is 1 to 2 to 5 and where Y′ is preferably Ahx. In one embodiment, Y′ is a 6-aminohexanoic acid subunit, R is arginine and p is 4. Certain embodiments include various linear combinations of at least two of (RY′R)ₚ and (RRY′)ₚ, including, for example, illustrative peptides having the sequence (RY′R)(RRY′)(RY′R)(RRY′) (SEQ ID NO:638), or (RRY′)(RY′R)(RRY′) (SEQ ID NO:639). Other combinations are contemplated. In a further illustrative embodiment, each Z′ is phenylalanine, and m is 3 or 4.

The conjugated peptide is preferably linked to a terminus of the oligomer via a linker Ahx-B, where Ahx is a 6-aminohexanoic acid subunit and B is a β-alanine subunit, as shown, for example, in FIGS. 1B and 1C.

In selected embodiments, for each X′, the side chain moiety is independently selected from the group consisting of guanidyl (HN═C(NH₂)NH—), amidinyl (HN═C(NH₂)CH—), 2-aminodihydropyrimidyl, 2-aminotetrahydropyrimidyl, 2-aminopyridinyl, and 2-aminopyrimidonyl, and it is preferably selected from guanidyl and amidinyl. In one embodiment, the side chain moiety is guanidyl, as in the amino acid subunit arginine (Arg).

In certain embodiments, the Y′ subunits may be contiguous, in that no X′ subunits intervene between Y′ subunits, or interspersed singly between X″ subunits. In certain embodiments, the linking subunit may be between Y′ subunits. In one embodiment, the Y′ subunits are at a terminus of the transporter; in other embodiments, they are flanked by X′ subunits. In further preferred embodiments, each Y′ is —C(═O)—(CH₂)_{n—}CHR—NH—, where n is 2 to 7 and R is H. For example, when n is 5 and R is H, Y′ is a 6-aminohexanoic acid subunit, abbreviated herein as Ahx. In selected embodiments of this group, each X′ comprises a guanidyl side chain moiety, as in an arginine subunit. Preferred peptides of this type include those comprising arginine dimers alternating with single Y′ subunits, where Y′ is preferably Ahx. Examples include peptides having the formula (RY′R)₄ or the formula (RRY′)₄ where Y′ is preferably Ahx. In the latter case, the nucleic acid analog is preferably linked to a terminal Y′ subunit, preferably at the C-terminus, as shown, for example, in FIGS. 1B and 1C. The preferred linker is of the structure AhxB, where Ahx is a 6-aminohexanoic acid subunit and B is a β-alanine subunit.

The transport moieties as described above have been shown to greatly enhance cell entry of attached oligomers, relative to uptake of the oligomer in the absence of the attached transport moiety, and relative to uptake by an attached transport moiety lacking the hydrophobic subunits Y′. Such enhanced uptake is preferably evidenced by at least a two-fold increase, and preferably a four-fold increase, in the uptake of the compound into mammalian cells relative to uptake of the agent by an attached transport moiety lacking the hydrophobic subunits Y′. Uptake is preferably enhanced at least twenty fold, and more preferably forty fold, relative to the unconjugated compound.

A further benefit of the transport moiety is its expected ability to stabilize a duplex between an antisense compound and its target nucleic acid sequence, presumably by virtue of electrostatic interaction between the positively charged transport moiety and the negatively charged nucleic acid. The number of charged subunits in the transporter is less than 14, as noted above, and preferably between 8 and 11, since too high a number of charged subunits may lead to a reduction in sequence specificity.

The use of arginine-rich peptide transporters (i.e., cell-penetrating peptides) is particularly useful in practicing the present invention. Certain peptide transporters have been shown to be highly effective at delivery of antisense compounds into primary cells including muscle cells (Marshall, Oda et al. 2007; Jearawiriyapaisarn, Moulton et al. 2008; Wu, Moulton et al. 2008). Furthermore, compared to other peptide transporters such as Penetratin and the Tat peptide, the peptide transporters described herein, when conjugated to an antisense PMO, demonstrate an enhanced ability to alter splicing of several gene transcripts (Marshall, Oda et al. 2007). Especially preferred are the P007, CP06062 and CP04057 transport peptides listed below in Table 3 (SEQ ID NOS: 573, 578 and 577, respectively).

Exemplary peptide transporters, including linkers (B or AhxB) are given below in Table B below. Preferred sequences are those designated CP06062 (SEQ ID NO: 578), P007 (SEQ ID NO: 573) and CP04057 (SEQ ID NO: 577).

TABLE B
Exemplary Peptide Transporters for Intracellular
Delivery of PMO

| | Sequence | SEQ ID |
|---|---|---|
| Peptide | (N-terminal to C-terminal) | NO: |
| rTAT | RRRQRRKKRC | 570 |
| R₉F₂ | RRRRRRRRRFFC | 571 |
| (RRAhx)₄B | RRAhxRRAhxRRAhxRRAhxB | 572 |
| (RAhxR)₄AhxB; | RAhxRRAhxRRAhxRRAhxRAhxB | 573 |
| (P007) | | |
| (AhxRR)₄AhxB | AhxRRAhxRRAhxRRAhxRRAhxB | 574 |
| (RAhx)₆B | RAhxRAhxRAhxRAhxRAhxRAhxB | 575 |
| (RAhx)₈B | RAhxRAhxRAhxRAhxRAhxRAhxR | 576 |
| | AhxB | |
| (RAhxR)₅AhxB | RAhxRRAhxRRAhxRRAhxRRAhxR | 577 |
| (CP05057) | AhxB | |
| (RAhxRRBR)₂AhxB; | RAhxRRBRRAhxRRBRAhxB | 578 |
| (CP06062) | | |
| MSP | ASSLNIA | 579 |

### Formulations

In certain embodiments, the present invention provides formulations or compositions suitable for the therapeutic delivery of antisense oligomers, as described herein. Hence, in certain embodiments, the present invention provides pharmaceutically acceptable compositions that comprise a therapeutically-effective amount of one or more of the oligomers described herein, formulated together with one or more pharmaceutically acceptable carriers (additives) and/or diluents. While it is possible for an oligomer of the present invention to be administered alone, it is preferable to administer the compound as a pharmaceutical formulation (composition).

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including the isolated oligomers of the present invention.

As detailed below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually: (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the antisense oligomers of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, DF et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The invention also features the use of the composition comprising surface-modified liposomes containing poly (ethylene glycol) lipids (PEG-modified, branched and unbranched or combinations thereof, or long-circulating liposomes or stealth liposomes). Oligomers of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the present invention includes oligomer compositions prepared for delivery as described in U.S. Pat. Nos. 6,692,911, 7,163,695 and 7,070,807. In this regard, in one embodiment, the present invention provides an oligomer of the present invention in a composition comprising copolymers of lysine and histidine (HK) as described in U.S. Pat. No. 7,163,695, 7,070,807, and 6,692,911 either alone or in combination with PEG (e.g., branched or unbranched PEG or a mixture of both), in combination with PEG and a targeting moiety or any of the foregoing in combination with a crosslinking agent. In certain embodiments, the present invention provides antisense oligomers in compositions comprising gluconic-acid-modified polyhistidine or gluconylated-polyhistidine/transferrin-polylysine. One skilled in the art will also recognize that amino acids with properties similar to His and Lys may be substituted within the composition.

Certain embodiments of the oligomers described herein may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the subject oligomers include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain embodiments, the oligomers of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g.,, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from cyclodextrins, celluloses, liposomes, micelle forming agents, e.g., bile acids, and polymeric carriers, e.g., polyesters and polyanhydrides; and an oligomer of the present invention. In certain embodiments, an aforementioned formulation renders orally bioavailable an oligomer of the present invention.

Methods of preparing these formulations or compositions include the step of bringing into association an oligomer of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. An oligomer of the present invention may also be administered as a bolus, electuary or paste.

In solid dosage forms of the invention for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an oligomer as provided herein include powders, sprays, ointments, pastes, creams, lotions, gels, solutions, patches and inhalants. The active oligomers may be mixed under sterile conditions with a pharmaceutically-acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an oligomer of the present invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an oligomer of the present invention to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more oligomers of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in tum, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the subject oligomers in biodegradable polymers such as polylactide-polyglycolide. Depending on the ratio of oligomer to polymer, and the nature of the particular polymer employed, the rate of oligomer release can be controlled. Examples of other biodegradable polymers include poly (orthoesters) and poly (anhydrides). Depot injectable formulations may also prepared by entrapping the drug in liposomes or microemulsions that are compatible with body tissues.

When the oligomers of the present invention are administered as pharmaceuticals, to humans and animals, they can be given per se or as a pharmaceutical composition containing, for example, 0.1 to 99% (more preferably, to 30%) of active ingredient in combination with a pharmaceutically acceptable carrier.

As noted above, the formulations or preparations of the present invention may be given orally, parenterally, topically, or rectally. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare. subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the oligomers of the present invention, which may be used in a suitable hydrated form, and/or the pharmaceutical compositions of the present invention, may be formulated into pharmaceutically-acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular oligomer of the present invention employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. In certain situations, dosing is one administration per day. In certain embodiments, dosing is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to maintain the desired expression of a functional dystrophin protein.

Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17 (12), 1685-1713, 1991 and REV 5901 (Sheen, P. C., et al., J Pharm Sci 80 (7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In one aspect of invention, the formulations contain micelles formed from an oligomer as provided herein and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated. the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di-and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In certain embodiments, the delivery may occur by use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, polymers have a molecular weight of from about 100 or 120 daltons up to about 5,000 or 10,000 daltons, or from about 300 daltons to about 5,000 daltons. In other embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG(750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present invention relates to formulations comprising liposomes containing an oligomer of the present invention, where the liposome membrane is formulated to provide a liposome with increased carrying capacity. Alternatively or in addition, the compound of the present invention may be contained within, or adsorbed onto, the liposome bilayer of the liposome. An oligomer of the present invention may be aggregated with a lipid surfactant and carried within the liposome's internal space; in these cases, the liposome membrane is formulated to resist the disruptive effects of the active agent-surfactant aggregate.

According to one embodiment of the present invention, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present invention are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPCs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present invention.

Liposomes according to the present invention may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993. For example, liposomes of the present invention may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT® and Lipofectamine® may be utilized to introduce polynucleotides or proteins into cells.

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

An oligomer may be formulated to be contained within, or, adapted to release by a surgical or medical device or implant. In certain aspects, an implant may be coated or otherwise treated with an oligomer. For example, hydrogels, or other polymers, such as biocompatible and/or biodegradable polymers, may be used to coat an implant with the compositions of the present invention (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the oligomers for use according to the invention may be formulated for administration in any convenient way for use in human or veterinary medicine, by analogy with other pharmaceuticals. The antisense oligomers and their corresponding formulations may be administered alone or in combination with other therapeutic strategies in the treatment of muscular dystrophy, such as myoblast transplantation, stem cell therapies, administration of aminoglycoside antibiotics, proteasome inhibitors, and up-regulation therapies (e.g., upregulation of utrophin, an autosomal paralogue of dystrophin).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### REFERENCES

Aartsma-Rus, A., A. A. Janson, et al. (2004). “Antisense-induced multiexon skipping for Duchenne muscular dystrophy makes more sense.” Am J Hum Genet 74 (1): 83-92.
Dunckley, M. G., I. C. Eperon, et al. (1997). “Modulation of splicing in the DMD gene by antisense oligoribonucleotides.” Nucleosides & Nucleotides 16 (7-9): 1665-1668.
Dunckley, M. G., M. Manoharan, et al. (1998). “Modification of splicing in the dystrophin gene in cultured Mdx muscle cells by antisense oligoribonucleotides.” Hum Mol Genet 7 (7): 1083-90.
Errington, S. J., C. J. Mann, et al. (2003). “Target selection for antisense oligonucleotide induced exon skipping in the dystrophin gene.” J Gene Med 5 (6): 518-27.
Jearawiriyapaisarn, N., H. M. Moulton, et al. (2008). “Sustained Dystrophin Expression Induced by Peptide-conjugated Morpholino Oligomers in the Muscles of mdx Mice.” Mol Ther.
Lu, Q. L., C. J. Mann, et al. (2003). “Functional amounts of dystrophin produced by skipping the mutated exon in the mdx dystrophic mouse.” Nat Med 9 (8): 1009-14.
Mann, C. J., K. Honeyman, et al. (2002). “Improved antisense oligonucleotide induced exon skipping in the mdx mouse model of muscular dystrophy.” J Gene Med 4 (6): 644-54.
Marshall, N. B., S. K. Oda, et al. (2007). “Arginine-rich cell-penetrating peptides facilitate delivery of antisense oligomers into murine leukocytes and alter pre-mRNA splicing.” Journal of Immunological Methods 325 (1-2): 114-126.
Matsuo, M., T. Masumura, et al. (1991). “Exon skipping during splicing of dystrophin mRNA precursor due to an intraexon deletion in the dystrophin gene of Duchenne muscular dystrophy kobe.” J Clin Invest 87 (6): 2127-31.
Monaco, A. P., C. J. Bertelson, et al. (1988). “An explanation for the phenotypic differences between patients bearing partial deletions of the DMD locus.” Genomics 2 (1): 90-5.
Pramono, Z. A., Y. Takeshima, et al. (1996). “Induction of exon skipping of the dystrophin transcript in lymphoblastoid cells by transfecting an antisense oligodeoxynucleotide complementary to an exon recognition sequence.” Biochem Biophys Res Commun 226 (2): 445-9.
Sazani, P., R. Kole, et al. (2007). Splice switching oligomers for the TNF superfamily receptors and their use in treatment of disease. PCT WO2007058894, University of North Carolina
Sierakowska, H., M. J. Sambade, et al. (1996). “Repair of thalassemic human beta-globin mRNA in mammalian cells by antisense oligonucleotides.” Proc Natl Acad Sci U S A 93 (23): 12840-4.
Summerton, J. and D. Weller (1997). “Morpholino antisense oligomers: design, preparation, and properties.” Antisense Nucleic Acid Drug Dev 7 (3): 187-95.
Takeshima, Y., H. Nishio, et al. (1995). “Modulation of in vitro splicing of the upstream intron by modifying an intra-exon sequence which is deleted from the dystrophin gene in dystrophin Kobe.” J Clin Invest 95 (2): 515-20.
van Deutekom, J. C., M. Bremmer-Bout, et al. (2001). “Antisense-induced exon skipping restores dystrophin expression in DMD patient derived muscle cells.” Hum Mol Genet 10 (15): 1547-54.
van Deutekom, J. C., A. A. Janson, et al. (2007). “Local dystrophin restoration with antisense oligonucleotide PRO051.” N Engl J Med. 357 (26): 2677-86.
Wilton, S. D., A. M. Fall, et al. (2007). “Antisense oligonucleotide-induced exon skipping across the human dystrophin gene transcript.” Mol Ther 15 (7): 1288-96.
Wilton, S. D., F. Lloyd, et al. (1999). “Specific removal of the nonsense mutation from the mdx dystrophin mRNA using antisense oligonucleotides.” Neuromuscul Disord 9 (5): 330-8.
Wu, B., H. M. Moulton, et al. (2008). “Effective rescue of dystrophin improves cardiac function in dystrophin-deficient mice by a modified morpholino oligomer.” Proc Natl Acad Sci U S A 105 (39): 14814-9.
Yin, H., H. M. Moulton, et al. (2008). “Cell-penetrating peptide-conjugated antisense oligonucleotides restore systemic muscle and cardiac dystrophin expression and function.” Hum Mol Genet 17 (24): 3909-18.

1. A composition for use in producing skipping of exon 44 in the processing of human dystrophin pre-processed mRNA, comprising a substantially uncharged antisense compound containing 20-35 morpholino subunits linked by phosphorus-containing intersubunit linkages joining a morpholino nitrogen of one subunit to a 5′ exocyclic carbon of an adjacent subunit, comprising a sequence selected from the group consisting SEQ ID NOS: 1-20, and capable of forming with the complementary mRNA sequence in the dystrophin-gene exon 44, a heteroduplex structure between said compound and mRNA having a Tm of at least 45° C.

2. The composition of claim 1, wherein the compound contains a sequence selected from the group consisting of SEQ ID NOS: 8, 11 and 12.

3. The composition of claim 1, wherein the compound is conjugated to an arginine-rich peptide having a sequence selected from the group consisting of SEQ ID NOS: 570-578.

4. A composition for use in producing skipping of exon 45 in the processing of human dystrophin pre-processed mRNA, comprising a substantially uncharged antisense compound containing 20-35 morpholino subunits linked by phosphorus-containing intersubunit linkages joining a morpholino nitrogen of one subunit to a 5′ exocyclic carbon of an adjacent subunit, comprising a sequence selected from the group consisting SEQ ID NOS: 21-76 and 612 to 624, and capable of forming with the complementary mRNA sequence in the dystrophin-gene exon 45, a heteroduplex structure between said compound and mRNA having a Tm of at least 45° C.

5. The composition of claim 4, wherein the compound contains a sequence selected from the group consisting of SEQ ID NOS: 27, 29, 34 and 39.

6. The composition of claim 5, wherein the compound contains a sequence selected from the group consisting of SEQ ID NOS: 29 and 34.

7. The composition of claim 4, wherein the compound is conjugated to an arginine-rich peptide having a sequence selected from the group consisting of SEQ ID NOS: 570-578.

8. A composition for use in producing skipping of exon 46 in the processing of human dystrophin pre-processed mRNA, comprising a substantially uncharged antisense compound containing 20-35 morpholino subunits linked by phosphorus-containing intersubunit linkages joining a morpholino nitrogen of one subunit to a 5′ exocyclic carbon of an adjacent subunit, comprising a sequence selected from the group consisting SEQ ID NOS: 77-125, and capable of forming with the complementary mRNA sequence in the dystrophin-gene exon 46, a heteroduplex structure between said compound and mRNA having a Tm of at least 45° C.

9. The composition of claim 8, wherein the compound contains a sequence selected from the group consisting of SEQ ID NOS: 77-105.

10. The composition of claim 9, wherein the compound contains a sequence selected from the group consisting of SEQ ID NOS: 82, 84-87, 90, 96, 98, 99 and 101.

11. The composition of claim 8, wherein the compound is conjugated to an arginine-rich peptide having a sequence selected from the group consisting of SEQ ID NOS: 570-578.

12-65. (canceled)
